(19) United States
(12) Patent Application Publication
(10) Pub. No.: US 2013/0137748 A1
(43) Pub. Date: May 30, 2013

(54) WHSC1 AND WHSC1L1 FOR TARGET GENES OF CANCER THERAPY AND DIAGNOSIS

(71) Applicants: Ryuji Hamamoto, Tokyo (JP); Yusuke Nakamura, Tokyo (JP); Takuya Tsunoda, Kanagawa (JP)

(72) Inventors: Ryuji Hamamoto, Tokyo (JP); Yusuke Nakamura, Tokyo (JP); Takuya Tsunoda, Kanagawa (JP)

(73) Assignee: OncoTherapy Science, Inc., Kanagawa (JP)

(21) Appl. No.: 13/576,917

(22) PCT Filed: Feb. 2, 2011
(86) PCT No.: PCT/JP11/00585

§ 371 Date: Feb. 4, 2013

Related U.S. Application Data
(60) Provisional application No. 61/301,025, filed on Feb. 3, 2010.
(60) Provisional application No. 61/411,689, filed on Nov. 9, 2010.

(51) Int. Cl. C12N 15/113; C12Q 1/02; G01N 33/53

(52) U.S. Cl. CPC C12N 15/1137; G01N 33/53
USPC 514/44 A; 536/24.5; 435/7.8; 435/6.13; 435/15; 435/320.1

(57) ABSTRACT

Objective methods for diagnosing a predisposition to developing cancer, for example, bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma, are described herein. In one embodiment, the diagnostic method involves determining an expression level of a WHSC1 or WHSC1L1 gene. The present invention further provides methods of screening for therapeutic agents useful in the treatment of WHSC1 or WHSC1L1 associated disease, such as a cancer, e.g., bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma. The present invention further provides methods of inhibiting the cell growth and treating or alleviating symptoms of WHSC1 or WHSC1L1 associated diseases. The present invention also features products, including double-stranded molecules and vectors encoding thereof as well as to compositions including them. Also, disclosed are methods of identifying substances for treating or/and preventing lung cancer, using as an index their effect on expression of a WHSC1 or WHSC1L1 gene, or a biological activity of a WHSC1 or WHSC1L1 polypeptide.

### PRIORITY

The present application claims priority to U.S. Ser. No. 61/301,025, filed Feb. 3, 2010, and U.S. Ser. No. 61/411,689, filed Nov. 9, 2010, the disclosures of which are incorporated herein by reference in their entireties.

### TECHNICAL FIELD

The present invention relates to methods of detecting and diagnosing a predisposition to developing cancer, particularly bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma. The present invention also relates to methods of screening for a candidate substance for treating and preventing cancer with over-expression of WHSC1 and/or WHSC1L1, particularly bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma. Moreover, the present invention relates to a double-stranded molecule which reduces WHSC1 or WHSC1L1 gene expression and uses thereof.

### BACKGROUND ART

The N-terminal tails of histones are subjected to post-translational modifications, including methylation, acetylation and phosphorylation, generating an extensive repertoire of chromatin structures (NPLs 1, 2). The present inventors previously reported that SMYD3, a histone lysine methyltransferase, stimulates proliferation of cells and plays an important role in human carcinogenesis through its methyl-transferase activity (PTL 1, NPLs 3-7). With the exception of Dot1/DOT1L, all histone lysine methyltransferases (HKMTs) contain a SET domain of about 130 amino acids (NPL 8). The SET domain was originally identified as a shared domain in three Drosophila proteins involved in epigenetic processes: the suppressor of position-effect variegation [Su(var)3-9]; an enhancer of the eye colour mutant zeste which belongs to the PcG proteins [E(Z)]; and the homeobox gene regulator trithorax [TRX] (NPL 9). Mammalian homologues of Drosophila Su(var)3-9, Suv39h1 and Suv39h2, were the first ones characterized as HKMTs and specifically methylate histone H3 at lysine 9 (H3K9) (NPL 10). So far, nearly 40 HKMTs or potential HKMTs containing SET domain have been identified and some of them are shown to methylate lysine residues at codons 4, 9, and 27 or 36 of histone H3 and lysine 20 of histone H4. The HKMTs can be classified into several different families according to sequence similarities within their SET domain and within the adjacent sequences, as well as based on other structural features such as the presence of other defined protein domain (NPL 8). Although information about histone methyltransferases and their physiological function has been accumulated, their involvement in human disease remains largely unclear.

In order to investigate possible roles of HKMTs in human carcinogenesis, the present inventors examined the expression profiles of human HKMTs in clinical tissues and found that expression levels of WHSC1 and WHSC1L1 were significantly up-regulated, compared with expression in corresponding normal tissues, in various types of cancer. WHSC1, also known as NSD2, was identified as a candidate gene for Wolf-Hirschhorn syndrome (WHS) (NPL 11). Through translocations t(4;14) (p16.3;q32.3) WHSC1 is indicated to be involved in multiple myeloma (NPLs 11, 12). The WHSC1 protein contains AWS-SET-ProSET domains that are highly conserved with yeast H3K36-specific methyltransferase Set 2. Mouse Whsc1 was recently reported to govern H3K36me3 distribution along euchromatin by associating with the cell-type-specific transcription factors Sall1, Sall4 and Nanog in embryonic stem cells (NPL 13). WHSC1L1, also known as NSD3 and WHISTLE, is related to the WHSC1 gene and encodes a protein with PWWP (proline-tryptophan-tryptophan-proline) domains (NPL 14). WHSC1L1 is located in chromosome 8p12 and shows strong sequence similarity to WHSC1 and NSD1, particularly in the 3′ region of the protein, which includes the functional domains (NPLs 14, 15). Although WHSC1L1 is known to be a transcriptional repressor through mediating histone methylation (NPLs 16, 17), the cellular function of the protein has not been determined.

### CITATION LIST

### Patent Literature

[PTL1] WO2005/071102

### Non-Patent Literature

[NPL 1] Jenuwein T and Allis CD Science 293: 1074-1080, 2001.
[NPL 2] Lachner M, et al. Nature 410: 116-120, 2001.
[NPL 3] Hamamoto R, et al. Nat Cell Biol 6: 731-740, 2004.
[NPL 4] Hamamoto R, et al. Cancer Sci 97: 113-118, 2006.
[NPL 5] Kunizaki M, et al. Cancer Res 67: 10759-10765, 2007.
[NPL 6] Silva F P, et al. Oncogene 27: 2686-2692, 2008.
[NPL 7] Tsuge M, et al. Nat Genet. 37: 1104-1107, 2005.
[NPL 8] Volkel P and Angrand P O. Biochimie 89: 1-20, 2007.
[NPL 9] Jenuwein T, et al. Cell Mol Life Sci 54: 80-93, 1998.
[NPL 10] Rea S, et al. Nature 406: 593-599, 2000.
[NPL 11] Stec I, et al. Hum Mol Genet. 7: 1071-1082, 1998
[NPL 12] Chesi M, et al. Blood 1998; 92:3025-34.
[NPL 13] Nimura K, et al. Genomics 2001; 76:5-8.
[NPL 14] Stec I, et al. Genomics 2001; 76:5-8.
[NPL 15] Douglas J, et al. Eur J Hum Genet. 2005; 13:150-3.
[NPL 16] Kim S M, et al. Biochem Biophys Res Commun 2006; 345:318-23.
[NPL 17] Kim S M, et al. Exp Cell Res 2007; 313:975-83.

### SUMMARY OF INVENTION

In order to investigate possible roles of HKMTs in human carcinogenesis, the present inventors examined the expression profiles of human HKMTs in clinical tissues and found that expression levels of WHSC1 and WHSC1L1 were significantly up-regulated, compared with their corresponding normal tissues, in various types of cancer.

In the present invention, it was identified that WHSC1 and WHSC1L1 are over-expressed in various types of human cancer. Since these genes are scarcely expressed in adult normal organs, WHSC1 and WHSC1L1 are appropriate molecular targets for novel therapeutic approaches with minimal adverse effect. Functionally, knockdown of endogenous WHSC1 or WHSC1L1 by siRNA in cancer cell lines resulted in drastic suppression of cancer cell growth, demonstrating an essential role for these genes in maintaining viability of cancer cells.

Accordingly, the present invention features a method of diagnosing or determining a predisposition to cancer, particularly bladder cancer, breast cancer, cholangiocellular carcinoma, chronic myeloid leukaemia (CML), esophageal cancer, hepatocellular carcinoma (HCC), non-small cell lung cancer (NSCLC), small cell lung cancer (SCLC), osteosarcoma, pancreatic cancer, prostate cancer, renal cell carcinoma, soft tissue tumor and lymphoma in a subject by determining expression levels of WHSC1 and/or WHSC1L1 gene in a subject-derived biological sample, such as biopsy sample or specimen. An increase of the expression level of WHSC1 and/or WHSC1L1 compared to a normal control level indicates that the subject suffers from or is at risk of developing cancer, particularly bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma. In the methods, transcripts (i.e., mRNAs) of WHSC1 and/or WHSC1L1 genes can be detected by appropriate probes or primer sets, or the WHSC1 and/or WHSC1L1 proteins can be detected by anti-WHSC1 or WHSC1L1 antibodies.

The present invention further provides methods of identifying substances that inhibit the expression of WHSC1 or WHSC1L1 genes or the activities of the gene products. Furthermore, the present invention provides methods of identifying a candidate substance for treating and/or preventing WHSC1 or WHSC1L1 associated-disease, such as cancer, e.g., bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor, lymphoma or a candidate substance that inhibits growth of cells over-expressing the WHSC1 and/or WHSC1L1 gene. A decrease in the expression level of the WHSC1 or WHSC1L1 gene and/or biological activity of these gene products (i.e., protein or polypeptide) as compared to that in the absence of the test substance indicates that the test substance is an inhibitor of the WHSC1 or WHSC1L1 and can be used to inhibit the growth of cells over-expressing the WHSC1 or WHSC1L1 gene, such as cancerous cells, e.g., in bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma. The biological activities of the WHSC1 gene product to be detected in such screening can be preferably methyltransferase activity, cell-proliferating activity (cell proliferation enhancing activity) or binding activity to IQGAP1, TIAM1, AKT2 or beta-catenin. The biological activity of the WHSC1L1 gene product to be detected in such screening can be preferably methyltransferase activity or cell-proliferating activity (cell proliferation enhancing activity).

In another aspect, the present invention provides a method for inhibiting the growth of a cancerous cell over-expressing WHSC1 and/or WHSC1L1 gene by administering a substance that inhibits the expression of WHSC1 or WHSC1L1 gene and/or function of the WHSC1 or WHSC1L1 protein. Preferably, the substance is an inhibitory nucleic acid (e.g., an antisense, ribozyme, double-stranded molecule). The substance can be a nucleic acid molecule or vector for providing double-stranded molecule. Expression of the gene can be inhibited by introduction of a double-stranded molecule into a target cell in an amount sufficient to inhibit the target gene. The present invention also provides methods for inhibiting the growth of cancerous cells over-expressing WHSC1 or WHSC1L1 in a subject. The present methods are useful for treating and/or preventing cancer, particularly bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma.

In another aspect, the present invention relates to a pharmaceutical composition for treating and/or preventing cancer that includes one or more double-stranded molecules or vectors encoding such molecules as active ingredients and a pharmaceutically acceptable carrier. Furthermore, the present invention also provides double-stranded molecules directed against a WHSC1 or WHSC1L1 gene and vectors encoding such molecules. The double-stranded molecules provided in the present invention inhibit expression of the WHSC1 or WHSC1L1 gene and inhibit the growth of cancerous cells over-expressing WHSC1 or WHSC1L1 when introduced into the cells. Preferably, such molecules target the nucleotide sequence corresponding to SEQ ID NO: 29 or 32 for the WHSC1 gene and SEQ ID NO: 35 or 38 for WHSC1L1 gene. The double-stranded molecules of the present invention include a sense strand and an antisense strand, wherein the sense strand includes a nucleotide sequence including the target sequence, and wherein the antisense strand includes a nucleotide sequence which is complementary to the target sequence. The sense and the antisense strands of the molecule hybridize to each other to form a double-stranded molecule.

Particularly, the present invention provides the following inventions:

[1] A method of detecting or diagnosing cancer in a subject, comprising determining an expression level of WHSC1 gene and/or an expression level of WHSC1L1 gene in a subject-derived biological sample (i.e., a sample obtained from the subject), wherein an increase of said level compared to a normal control level of said gene indicates that said subject suffers from or is at risk of developing cancer, wherein the expression level is determined by a method selected from the group consisting of:

(a) detecting an mRNA of WHSC1 and/or an mRNA of WHSC1L1;

(b) detecting a protein encoded by WHSC1 gene and/or a protein encoded by WHSC1L1 gene; and

(c) detecting a biological activity of a protein encoded by WHSC1 and/or a biological activity of a protein encoded by WHSC1L1 gene;

[2] The method of [1], wherein said increase is at least 10% greater than said normal control level;

[3] The method of [1], wherein the subject-derived biological sample is biopsy or blood sample;

[4] A kit for diagnosing cancer, which comprises at least one reagent selected from the group consisting of:

(a) a reagent for detecting mRNA of WHSC1 or WHSC1L1;

(b) a reagent for detecting protein encoded by a WHSC1 or WHSC1L1 gene; and

(c) a reagent for detecting a biological activity of the protein encoded by a WHSC1 or WHSC1L1 gene;

[5] The kit of [4], wherein the reagent is selected from the group consisting of:

(a) a probe to an mRNA of WHSC1 or WHSC1L1; and

(b) an antibody against a protein encoded by a WHSC1 or WHSC1L1 gene;

[6] A method of screening for a candidate substance for treating or preventing cancer, or inhibiting cancer cell growth, said method comprising steps of:

(a) contacting a test substance with a polypeptide encoded by a WHSC1 or WHSC1L1 gene;

(b) detecting binding activity between the polypeptide and the test substance; and

(c) selecting the test substance that binds to the polypeptide;

[7] A method of screening for a candidate substance for treating or preventing cancer, or inhibiting cancer cell growth, said method comprising steps of:

(a) contacting a test substance with a cell expressing a WHSC1 or WHSC1L1 gene;

(b) detecting the expression level of the WHSC1 or WHSC1L1 gene; and

(b) selecting the test substance that reduces the expression level of the WHSC1 or WHSC1L1 gene in comparison with the expression level detected in the absence of the test substance;

[8] A method of screening for a candidate substance for treating or preventing cancer or inhibiting cancer cell growth, said method comprising the steps of:

(a) contacting a test substance with a polypeptide encoded by a WHSC1 or WHSC1L1 gene;

(b) detecting biological activity of the polypeptide of step (a); and

(c) selecting the test substance that suppresses the biological activity of the polypeptide in comparison with the biological activity detected in the absence of the test substance;

[9] The method of [8], wherein the biological activity is cell proliferative activity, methyltransferase activity or binding activity to IQGAP1 polypeptide, TIAM1 polypeptide, AKT2 polypeptide or beta-catenin polypeptide;

[10] A method of screening for a candidate substance for treating or preventing cancer, or inhibiting cancer cell growth, said method comprising the steps of:

(a) contacting a test substance with a cell into which a vector comprising transcriptional regulatory region of WHSC1 or WHSC1L1 gene and a reporter gene that is expressed under control of the transcriptional regulatory region has been introduced,

(b) measuring the expression or activity of said reporter gene; and

(c) selecting the test substance that reduces the expression or activity of said reporter gene, as compared to the expression or activity in the absence of the test substance;

[11] A method of screening for a candidate substance for treating or preventing cancer, or inhibiting cancer cell growth, said method comprising the steps of:

(a) contacting an IQGAP1 polypeptide, TIAM1 polypeptide, AKT2 polypeptide and/or beta-catenin polypeptide, or functional equivalent thereof with a WHSC1 polypeptide or functional equivalent thereof in the presence of a test substance;

(b) detecting the binding between the IQGAP1 polypeptide, TIAM1 polypeptide, AKT2 polypeptide and/or beta-catenin polypeptide, and the WHSC1 polypeptide of step (a); and

(c) selecting the test substance that inhibits the binding between the IQGAP1 polypeptide, TIAM1 polypeptide, AKT2 polypeptide and/or beta-catenin polypeptide, and the WHSC1 polypeptide as compared to the binding detected in the absence of the test substance;

[12] A double-stranded molecule comprising a sense strand and an antisense strand, wherein the sense strand comprises a nucleotide sequence corresponding to a target sequence selected from the group consisting of SEQ ID NO: 29, 32, 35 and 38, and wherein the antisense strand comprises a nucleotide sequence which is complementary to the target sequence, wherein said sense strand and said antisense strand hybridize to each other to form said double-stranded molecule, and wherein said double-stranded molecule, when introduced into a cell expressing the WHSC1 or WHSC1L1 gene, inhibits expression of said gene;

[13] The double-stranded molecule of [12], wherein the double-stranded molecule is between about 19 and about 25 nucleotides in length;

[14] The double-stranded molecule of [12] or [13], wherein said double-stranded molecule is a single polynucleotide molecule comprising the sense strand and the antisense strand linked via a single-stranded nucleotide sequence;

[15] The double-stranded molecule of [14], wherein said polynucleotide has the general formula of

5′-[A]-[B]-[A′]-3′,

wherein [A] is a sense strand comprising a nucleotide sequence corresponding to a target sequence selected from the group consisting of SEQ ID NO: 29, 32, 35 and 38; [B] is a nucleotide sequence consisting of about 3 to about 23 nucleotides; and [A′] is an antisense strand comprising a nucleotide sequence complementary to the target sequence;

[16] A vector encoding the double-stranded molecule of any one of [12] to [15];

[17] A vector comprising each of a combination of polynucleotide, comprising a sense strand nucleic acid and an antisense strand nucleic acid, wherein said sense strand nucleic acid comprises a nucleotide sequence selected from the group consisting of SEQ ID NOs: 29, 32, 35 and 38, and said antisense strand nucleic acid consists of a sequence complementary to the sense strand, wherein the transcripts of said sense strand and said antisense strand hybridize to each other to form a double-stranded molecule, and wherein said vector inhibits expression of a target gene;

[18] A method of treating or preventing cancer in a subject, comprising administering to said subject a pharmaceutically effective amount of a double-stranded molecule directed against a WHSC1 or WHSC1L1 gene or a vector encoding said double-stranded molecule, and a pharmaceutically acceptable carrier, wherein the double-stranded molecule inhibits cell proliferation and expression of the WHSC1 or WHSC1L1 gene when introduced into a cell expressing the WHSC1 or WHSC1L1 gene;

[19] The method of [18], wherein the double-stranded molecule is that of any one of [12] to [15];
[20] The method of [18], wherein the vector is that of [16] or [17];
[21] A composition for treating or preventing cancer, which comprises a pharmaceutically effective amount of a double-stranded molecule directed against a WHSC1 or WHSC1L1 gene or a vector encoding said double-stranded molecule, and a pharmaceutically acceptable carrier, wherein the double-stranded molecule inhibits cell proliferation as well as expression of the WHSC1 or WHSC1L1 gene when introduced into a cell expressing the WHSC1 or WHSC1L1 gene;

[22] The composition of [21], wherein the double-stranded molecule is that of any one of [12] or [15];

[23] The composition of [21], wherein the vector is that of [16] or [17]; and

[24] A method of screening for a substance for inhibiting the binding between WHSC1 polypeptide or functional equivalent thereof and at least one of polypeptides selected from the group consisting of IQGAP1 polypeptide, TIAM1 polypeptide, AKT2 polypeptide or beta-catenin polypeptide or functional equivalent thereof, said method comprising the steps of:

(a) contacting an IQGAP1 polypeptide, TIAM1 polypeptide, AKT2 polypeptide or beta-catenin polypeptide, or functional equivalent thereof with a WHSC1 polypeptide or functional equivalent thereof in the presence of a test substance;

(b) detecting the binding between the polypeptides of step (a); and

(c) selecting the test substance that inhibits the binding between the IQGAP1 polypeptide, TIAM1 polypeptide, AKT2 polypeptide or beta-catenin polypeptide and the WHSC1 polypeptide.

In another embodiment, [1] a method of detecting or diagnosing cancer in a subject, comprising determining either of an expression level of WHSC1 gene or an expression level of WHSC1L1 gene, or both in a subject-derived biological sample (i.e., a sample obtained from the subject), wherein an increase of said level compared to a normal control level of said gene indicates that said subject suffers from or is at risk of developing cancer, wherein the expression level is determined by a method selected from the group consisting of:

(a) detecting either of an mRNA of WHSC1 or an mRNA of WHSC1L1, or both;

(b) detecting either of a protein encoded by WHSC1 gene or a protein encoded by WHSC1L1 gene, or both;

(c) detecting either of a biological activity of a protein encoded by WHSC1 or a biological activity of a protein encoded by WHSC1L1 gene, or both is provided.

In addition, in another embodiment, [7] a method of screening for a candidate substance for treating or preventing cancer or inhibiting cancer cell growth, said method comprising steps of:

(a) contacting a test substance with a cell expressing either or both of a WHSC1 and WHSC1L1 gene;
(b) detecting either of the expression level of the WHSC1 gene or the expression level of the WHSC1L1 gene, or both; and
(c) selecting the test substance that reduces either of the expression level of the WHSC1 gene or the expression level of the WHSC1L1 gene, or both in comparison with the expression level detected in the absence of the test substance is also provided. In addition, in another embodiment, [11] a method of screening for a candidate substance for treating or preventing cancer, said method comprising the steps of:
(a) contacting at least one of polypeptides selected from the group consisting of an IQGAP1 polypeptide, TIAM1 polypeptide, AKT2 polypeptide and beta-catenin polypeptide, or functional equivalent thereof with a WHSC1 polypeptide or functional equivalent thereof in the presence of a test substance,
(b) detecting the binding between at least one of the polypeptides selected from the group consisting of IQGAP1 polypeptide, TIAM1 polypeptide, AKT2 polypeptide and beta-catenin polypeptide, and the WHSC1 polypeptide of step (a), and
(c) selecting the test substance that inhibits the binding between at least one of the polypeptides selected from the group consisting of IQGAP1 polypeptide, TIAM1 polypeptide, AKT2 polypeptide and beta-catenin polypeptide, and the WHSC1 polypeptide as compared to the binding detected in the absence of the test substance is also provided.

In addition, in another embodiment, [24] a method of screening for a substance for inhibiting the binding between WHSC1 polypeptide or functional equivalent thereof and at least one of polypeptides selected from the group consisting of IQGAP1 polypeptide, TIAM1 polypeptide, AKT2 polypeptide or beta-catenin polypeptide or functional equivalent thereof, said method comprising the steps of:

(a) contacting at least one of polypeptides selected from the group consisting of an IQGAP1 polypeptide, TIAM1 polypeptide, AKT2 polypeptide and beta-catenin polypeptide, or functional equivalent thereof with a WHSC1 polypeptide or functional equivalent thereof in the presence of a test substance;

(b) detecting the binding between at least one of the polypeptides selected from the group consisting of IQGAP1 polypeptide, TIAM1 polypeptide, AKT2 polypeptide and beta-catenin polypeptide, or the or functional equivalent thereof and the WHSC1 polypeptide, or the functional equivalent thereof; and

(c) selecting the test substance that inhibits the binding between at least one of the polypeptides selected from the group consisting of IQGAP1 polypeptide, TIAM1 polypeptide, AKT2 polypeptide and beta-catenin polypeptide or the functional equivalent thereof, and the WHSC1 polypeptide or the functional equivalent thereof as compared to the binding in the absence of the test substance is also provided.

### BRIEF DESCRIPTION OF DRAWINGS

[FIG. 1A-B]FIG. 1 depicts the graphs and the photographs showing the elevated WHSC1 expression in human cancers. (A) Expression levels of WHSC1 were analyzed by quantitative real-time PCR, and the result is shown by box-whisker plot (median 50% boxed). Relative mRNA expression shows the value normalized by GAPDH and SDH expressions. Mann-Whitney U test was used for statistical analysis. (B) Immunohistochemical staining of WHSC1 and WHSC1L1 in normal and cancer bladder tissues. Counterstaining was done with hematoxylin and eosin. Original magnification, ×100 and ×400.

[FIG. 1C-D](C) Comparison of WHSC1 expression between normal and tumor tissues. Signal intensity of each sample was analyzed by cDNA microarray, and the result is shown by box-whisker plot (median 50% boxed). Mann-Whitney U test was used for the statistical analysis. (D) Comparison of WHSC1L1 expression between normal and tumor tissues in chronic myelogenous leukemia, lymphoma, lung cancer (SCLC) and breast cancer. Signal intensity of each sample was analyzed by cDNA microarray, and the result is shown by box-whisker plot (median 50% boxed). Mann-Whitney U test was used for the statistical analysis.

FIG. 2 depicts tissue microarray images of bladder tissues stained by standard immunohistochemistry for protein expression of WHSC1. Clinical information for each section is represented above histological pictures. Counterstaining was done with hematoxylin and eosin. Original magnification, ×400.

FIG. 3A depicts tissue microarray images of lung tissues stained by standard immunohistochemistry for protein expression of WHSC1. Clinical information for each section is represented above histological pictures. Counterstaining was done with hematoxylin and eosin. Original magnification, ×400.

FIG. 3B depicts tissue microarray images of lung tissues stained by standard immunohistochemistry for protein expression of WHSC1. Clinical information for each section is represented above histological pictures. Counterstaining was done with hematoxylin and eosin. Original magnification, ×400.

FIG. 3C-D depicts tissue microarray images of lung tissues stained by standard immunohistochemistry for protein expression of WHSC1. Clinical information for each section is represented above histological pictures. Counterstaining was done with hematoxylin and eosin. Original magnification, ×400.

[FIG. 4A-B]FIG. 4 depicts the graphs showing the involvement of WHSC1 and WHSC1L1 in the growth of bladder and lung cancer cells. (A) Quantitative real-time PCR showing suppression of endogenous expression of WHSC1 by two WHSC1-specific siRNAs (siWHSC1#1 and #2) in SW780 cells and two WHSC1L1-specific siRNAs (siWHSC1L1#1 and #2) in A549 cells. siRNAs targeting EGFP (siEGFP) and siNegative control (siNC) were used as controls. mRNA expression levels were normalized by GAPDH and SDH expressions, and values are relative to siEGFP (siEGFP=1). Results are the mean+/−SD of three independent experiments. P values were calculated using Student's t-test (***, P<0.001). (B) Effects of WHSC1 and WHSC1L1 siRNA knockdown on the viability of two bladder cancer cell line (SW780, RT4) and three lung cancer cell lines (A549, LC319 and SBC5). Relative cell number shows the value normalized to siEGFP-treated cells (siEGFP=1). Results are the mean+/−SD in three independent experiments. P values were calculated using Student's t-test (*, P<0.05; **, P<0.01; ***, P<0.001).

[FIG. 4C](C) Effect of siWHSC1 on cell cycle kinetics in A549 and SW780 cells. Cell cycle distribution was analyzed by flow cytometry after coupled staining with flu-orescein isothiocyanate (FITC)-conjugated anti-BrdU and 7-amino-actinomycin D (7-AAD).

[FIG. 5A]FIG. 5 depicts that WHSC1 interacted with IQGAP1, TIAM1, AKT2 and beta-catenin. (A) Two-dimensional, unsupervised hierarchical cluster analysis of SW780 and A549 mRNA expression profiles after knockdown of WHSC1 expression. Differentially expressed genes were selected for this analysis. Red, Up-regulated; Green, Down-regulated.

[FIG. 5B-D](B) Silver staining pattern of interacting proteins with WHSC1. pCAGGS-n3FH-WHSC1 vectors were transfected into 293T cells. After 48 h, interacting protein partners of WHSC1 were enriched by anti-FLAG immunoprecipitation, separated by SDS-PAGE and silver stained. The different bands compared with a control lane were cut out and identified by mass spectrometry. Western blot was performed to confirm the expression of FLAG-WHSC1 using anti-FLAG antibody. (C) Immunoprecipitants from lysates of 293T cells using anti-FLAG M2 agarose (SIGMA) were immunoblotted with anti-FLAG (WHSC1), IQGAP1, TIAM1, AKT2 and beta-catenin antibodies. (D). HA-beta-catenin and 3×FLAG-WHSC1 vectores were transfected into 293T cells, and cell lysates were fractionated by NE-PER nuclear and cytoplasmic ex-traction kit (Thermo Sciecntific). Immunoprecipitants from fractionated lysates using anti-HA agarose (SIGMA) were immunoblotted with anti-FLAG and HA antibodies. UHRF1 was used as a marker of a nuclear protein, and Rho A was used a marker of a cytoplasmic protein.

[FIG. 5E](E), Immunocytochemical analysis of HCT116 cells after transfection with FLAG-tagged WHSC1. Cells were stained with anti-FLAG (Alexa Fluor (registered trademark) 488 [green]), anti-beta-catenin or anti-active-beta-catenin antibodies (Alexa Fluor (registered trademark) 594 [red]) and 4′,6′-diamidine-2′-phenylindole dihy-drochloride (DAPI [blue]).

FIG. 6 depicts that WHSC1 regulates the Wnt signaling pathway. (A) TOPFLAH and FOPFLASH analyses in 293T cells after transfection with the pCAGGS-WHSC1 vector. The mock vector was used as a control. Results are the mean+/−SD in three independent experiments, and the P-value was calculated using Student's t-test (**, P<0.01). (B) Signal intensity of CCND1 in SW780 and A549 cells after treatment with siEGFP (control) and siWHSC1 was quantified by GeneChip U133 plus 2.0 (Affymetrix). (C) Relative CCND1 mRNA levels in A549 and SBC5 cells after treatment with siEGFP (control) and siWHSC1 were analyzed by quantitative real-time PCR. Results are the mean+/−SD in three independent experiments, and the P-values were calculated using Student's t-test (**, P<0.01).

FIG. 7 depicts expression levels of WHSC1 and WHSC1L1 in 2 normal cell lines, 14 bladder cancer cell lines, 5 lung cancer cell lines, 3 liver cancer cell lines and 3 colon cancer cell lines. Expression levels were analyzed by quantitative real-time PCR, and relative mRNA expression shows the value normalized by GAPDH and SDH expressions.

FIG. 8 depicts expression levels of WHSC1 and WHSC1L1 in 29 normal tissues. Signal intensity was quantified by cDNA microarray. GAPDH expression is shown as a control of the signal intensity.

FIG. 9 depicts expression levels of WHSC1 and WHSC1L1 in 78 normal tissues. The data were derived from BioGPS (http://biogps.gnf.org/#goto=genereport&id=54904). GAPDH expression is shown as a control of the signal intensity.

FIG. 10 depicts the correlation between WHSC1 expression and the prognosis of lung cancer. (A) Representative cases for positive WHSC1 expression in lung ADC, SCC tissues and normal lung tissues. Original magnification, ×100 and ×200. (B) Kaplan-Meier estimates of overall survival time of patients with NSCLC(P=0.8629, log-rank test). (C) Positive ratio of WHSC1 in 328 lung tumor tissues.

FIG. 11 depicts chromatin immunoprecipitation (ChIP) assay for WHSC1wt and WHSC1[delta]SET at the promoter region of CCND1 gene. Top panel depicts a schematic diagram of the CCND1 promoter region. PCR amplified fragments are positioned by nucleotide number relatives to TSS (arrows). Middle panel depicts the confirmation of WHSC1wt and WHSC1[delta]SET protein expressions. The input samples were fractionated by SDS-PAGE and immunoblotted with anti-FLAG antibody. Expression of ACTB was the internal control. Bottom left panel depicts real-time PCR analysis using primer pairs as described under “Example 1”. Cross-linked and sheared chromatin was immunoprecipiated with anti-FLAG antibody (M2, Sigma). The results are shown as a percentage of the input chromatin. Bottom right panel depicts quantification of H3K36triMe ChIP at the CCND1 promoter region using real-time PCR. Cross-linked and sheared chromatin was immunoprecipiated with anti-triMeH3K36 antibody (ab9050, abcam).

FIG. 12 depicts TOPFLAH and FOPFLASH analyses in 293T cells after transfection with mock, WHSC1 wt and WHSC1[delta]SET vectors. The mock vector was used as a control. Results are the mean+/−SD in three independent experiments, and the P-value was calculated using Student's t-test (*, P<0.05; **, P<0.01).

FIG. 13 depicts a proposed model of WHSC1-mediated enhancement of beta-catenin/TCF-4-dependent transcription through histone H3 at lysine 36 tri-methylation.

FIG. 14 depicts the knockdown effect of beta-catenin on the growth of bladder and lung cancer cells. (A) Expression levels of beta-catenin in HCT116, A549, SBC5 and SW780 cells analyzed by quantitative real-time PCR. mRNA expression levels were normalized by GAPDH and SDH expressions, and values are relative to HCT116 (HCT116=1). (B) Quantitative real-time PCR showing suppression of endogenous expression of beta-catenin after treatment with specific siRNA in A549 cells. siEGFP and siNC were used as controls. mRNA expression levels were normalized by GAPDH and SDH expressions, and values are relative to siEGFP (siEGFP=1). (C) Effects of beta-catenin knockdown on the viability of bladder and lung cancer cell lines. Relative cell number shows the value normalized to siEGFP-treated cells. Results are the mean+/−SD in three independent experiments. P-values were calculated using Student's t-test (*, P<0.05).

### DESCRIPTION OF EMBODIMENTS

Before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

Unless otherwise defined, all technical and scientific terms used herein have the same meaning as commonly understood by one of ordinary skill in the art to which this invention belongs. In case of conflict, the present specification, including definitions, will control.

### DEFINITION

The words “a”, “an”, and “the” as used herein mean “at least one” unless otherwise specifically indicated.

The terms “isolated” and “purified” used in relation with a substance (e.g., polypeptide, antibody, polynucleotide, etc.) indicates that the substance is substantially free of at least one substance that may be included in the natural source. Thus, an isolated or purified antibody refers to antibodies that are substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which the polypeptide is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies and polypeptides of the present invention are isolated or purified. An “isolated” or “purified” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residues are a modified residue, or a non-naturally occurring residue, such as an artificial chemical mimetic of a corresponding naturally occurring amino acid, as well as to naturally occurring amino acid polymers. The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “polynucleotide”, “oligonucleotide”, and “nucleic acid” are used inter-changeably unless otherwise specifically indicated, to refer to a polymer of nucleotide residues. The terms apply to nucleotide polymers in which one or more nucleotide residues are modified residue, or a non-naturally occurring residue, such as an artificial chemical mimetic of a corresponding naturally occurring nucleotide, as well as to naturally occurring nucleotide polymers. The polynucleotide, oligonucleotide or nucleic acid can be composed of DNA, RNA or a combination thereof.

The term “nucleotide” refers to, similarly to the amino acid, naturally occurring and non-naturally occurring nucleotides. Similar to the amino acids, nucleotides, are referred to by their commonly accepted single-letter codes.

Unless otherwise defined, the term “cancer” refers to any cancer over-expressing the WHSC1 and/or WHSC1L1 gene, or either of WHSC1 and WHSC1L1 gene, or both. Examples of cancer over-expressing WHSC1 gene include, but are not limited to, bladder cancer, breast cancer, cholangiocellular carcinoma, chronic myelogenous leukemia (CML), esophageal cancer, hepatocellular carcinoma (HCC), non small cell lung carcinoma (NSCLC), SCLC (small cell lung carcinoma), osteosarcoma, pancreatic cancer, prostate cancer, renal cell carcinoma and soft tissue tumor. Examples of cancer over-expressing WHSC1L1 gene include, but are not limited to, bladder cancer, breast cancer, CML, Lymphoma and lung cancer (in particular, SCLC).

The term “specifically inhibit” in the context of inhibitory polynucleotides and polypeptides refers to the ability of an agent or ligand to inhibit the expression or the biological function of WHSC1 or WHSC1L1. Specific inhibition typically results in at least about a 2-fold inhibition over background, preferably greater than about 10 fold and most preferably greater than 100-fold inhibition of WHSC1 or WHSC1L1 expression (e.g., transcription or translation) or measured biological function (e.g., cell growth or proliferation, inhibition of apoptosis). Either of expression levels and biological function, or both can be measured in the context of comparing treated and untreated cells, or a cell population before and after treatment. In some embodiments, the expression or biological function of WHSC1 or WHSC1L1 is completely inhibited. Typically, specific inhibition is a statistically meaningful reduction in WHSC1 or WHSC1L1 expression or biological function (e.g., p<=0.05) using an appropriate statistical test.

Genes and Proteins of WHSC1 and WHSC1L1:

The present invention is based in part on the discovery that the genes encoding WHSC1 and WHSC1L1 are over-expressed in several cancers compared to non-cancerous tissue.

WHSC1 (Wolf-Hirschhorn syndrome candidate-1), a protein encoded by one of several genes in the identified Wolf-Hirschhorn syndrome (WHS) critical region, is deleted in every known case of WHS and is dysregulated by t(4;14) translocations in lymphoid multiple myeloma (Bergemann A D, et al. Trends Genet. 21: 188-195, 2005, Stec I, et al. Hum Mol Genet. 7: 1071-1082, 1998). The WHSC1 protein contains AWS-SET-ProSET domains that are highly conserved with yeast H3K36-specific methyltransferase Set 2 (Sun X J, et al. J Biol Chem 280: 35261-35271, 2005).

WHSC1L1 (Wolf-Hirschhorn syndrome candidate 1-like 1) is related to the WHSC1 gene and encodes a protein with PWWP (proline-tryptophan-tryptophan-proline) domains (Stec I, van Ommen G J, den Dunnen J T. Genomics 2001; 76:5-8). WHSC1L1 is located in chromosome 8p12 and shows strong sequence similarity to WHSC1 and NSD1, particularly in the 3′ region of the protein, which includes the functional domains (Stec I, van Ommen G J, den Dunnen J T. Genomics 2001; 76:5-8; Douglas J, Coleman K, Tatton-Brown K, et al. Eur J Hum Genet. 2005; 13:150-3).

An exemplary polypeptide and nucleic acid sequence of WHSC1 are shown in SEQ ID NO: 2 and 1, respectively. Also, an exemplary polypeptide and nucleic acid sequence of WHSC1L1 are shown in SEQ ID NOs: 4 or 50 and 3 or 49, respectively. The sequence data of WHSC1 and WHSC1L1 are also available via the following GenBank™ accession numbers:

WHSC1: NM_{—}001042424.2, NM_{—}007331.1, NM_{—}133330.2, NM_{—}133331.2, NM_{—}133334.2, NM_{—}133335.3 (the entire disclosures of which are herein incorporated by reference),

WHSC1L1: NM_{—}017778.2, NM_{—}023034.1 (the entire disclosures of which are herein incorporated by reference).

According to an aspect of the present invention, functional equivalents are also considered “WHSC1 polypeptides” or “WHSC1L1 polypeptides”. Herein, a “functional equivalent” of a protein (e.g., a WHSC1 polypeptide or WHSC1L1 polypeptide) is a polypeptide that has a biological activity equivalent to the protein. Namely, any polypeptide that retains the biological ability of the WHSC1 protein or WHSC1L1 protein can be used as such a functional equivalent in the present invention. WHSC1 and WHSC1L1 are known to have histone methyltransferase activity. Accordingly, functional equivalents of WHSC1 protein and WHSC1L1 protein preferably retain histone methyltransferase activity. Further, the results disclosed in Examples demonstrate that WHSC1 and WHSC1L1 have cell proliferating activity (cell proliferation enhancing activity). Therefore, functional equivalents of those proteins retain cell proliferating activity. Moreover, functional equivalents of WHSC1 protein can retain binding activity to IQGAP1, TIAM1, AKT2 or beta-catenin. In a preferred embodiment, functional equivalents of WHSC1 protein or WHSC1L1 protein retain one or more of the aforementioned biological activities of WHSC1 protein or WHC1L1 protein.

For example, preferred examples of functional equivalents of WHSC1 protein include polypeptides containing the SET domain of WHSC1 protein (e.g., 1066-1179 of SEQ ID NO: 2). Also, preferred examples of functional equivalents of WHSC1L1 protein include polypeptides containing the SET domain of WHSC1L1 protein (e.g., 1148 to 1261 of SEQ ID NO: 50).

Functional equivalents of WHSC1 protein or WHSC1L1 protein include those wherein one or more amino acids are substituted, deleted, added, or inserted to the natural occurring amino acid sequence of the WHSC1 protein or the WHSC1L1 protein. Alternatively, the polypeptide comprises an amino acid sequence having at least about 80% homology (also referred to as sequence identity) to the sequence of the respective protein, more preferably at least about 90% to 95% homology, often about 96%, 97%, 98% or 99% homology. In other embodiments, the polypeptide can be encoded by a polynucleotide that hybridizes under stringent conditions to the natural occurring nucleotide sequence of the WHSC1 gene or the WHSC1L1 gene.

A polypeptide of the present invention can have variations in amino acid sequence, molecular weight, isoelectric point, the presence or absence of sugar chains, or form, depending on the cell or host used to produce it or the purification method utilized. Nevertheless, so long as it has a function equivalent to that of the human WHSC1 protein or WHSC1L1 protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions can also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the human WHSC1 or WHSC1L1 protein can be routinely selected by a person skilled in the art. For example, hybridization can be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition can include 42 degrees C., 2×SSC, 0.1% SDS, preferably 50 degrees C., 2×SSC, 0.1% SDS. High stringency conditions are often preferably used. An exemplary high stringency condition can include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Generally, it is known that modifications of one or more amino acids in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that at least one mutation or alteration selected from the group consisting of individual additions, deletions, insertions, and substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity of the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or fewer, preferably 20 amino acids or fewer, more preferably 10 amino acids or fewer, more preferably 6 amino acids or fewer, and even more preferably 3 amino acids or fewer.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing sidechain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the WHSC1 protein or WHSC1L1 protein used in the present invention. However, the present invention is not restricted thereto and the WHSC1 protein or WHSC1L1 protein includes non-conservative modifications, so long as at least one biological activity of the WHSC1 protein or WHSC1L1 protein is retained. Furthermore, the modified proteins do not exclude polymorphic variants, interspecies homologues, and those encoded by alleles of these proteins.

Moreover, the WHSC1 gene or WHSC1L1 gene of the present invention encompasses polynucleotides that encode such functional equivalents of the WHSC1 protein or WHSC1L1 protein, respectively. In addition to hybridization, a gene amplification method, for example, the polymerase chain reaction (PCR) method, can be utilized to isolate a polynucleotide encoding a polypeptide functionally equivalent to the WHSC1 protein or WHSC1L1 protein, using a primer synthesized based on the sequence information of the protein encoding DNA (SEQ ID NO: 1, or 3 or 49). Polynucleotides and polypeptides that are functionally equivalent to the human WHSC1 and WHSC1L1 gene and protein, respectively, normally have a high homology to the originating nucleotide or amino acid sequence thereof. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

A Method for Diagnosing Cancer:

As disclosed herein, the expression levels of WHSC1 gene were found to be specifically elevated in several cancers, including bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor (FIG. 1, 2, 3, Table 5). Further, the expression levels of WHSC1L1 gene were also found to be specifically elevated in several cancers, including bladder cancer, CML, lung cancer (e.g., SCLC), breast cancer and lymphoma (FIG. 1, Table 5).

Therefore, WHSC1 and WHSC1L1 genes identified herein as well as their transcription and translation products find diagnostic utility as a marker for cancers such as above cancers. Diagnosing or detecting cancer in a subject can be conducted by determining the expression level of WHSC1 and/or WHSC1L1 gene in a subject-derived sample and comparing such expression level with the expression level detected in a normal sample. In preferred embodiments, cancers to be diagnosed or detected include bladder cancer, breast cancer, cholangiocellular carcinoma, CML, esophageal cancer, HCC, NSCLC (e.g., lung adenocarcinoma, lung squamous cell carcinoma (SCC)), SCLC, osteosarcoma, pancreatic cancer, prostate cancer, renal cell carcinoma, soft tissue tumor and lymphoma.

Alternatively, the present invention provides a method for detecting or identifying cancer cells in a subject-derived tissue sample, the method including the step of determining the expression level of the WHSC1 and/or WHSC1L1 gene in a subject-derived biological sample (i.e., a sample obtained from the subject), wherein an increase in the expression level as compared to a normal control level of the gene indicates the presence or suspicion of cancer cells in the tissue.

According to the present invention, an intermediate result for examining the condition of a subject can be provided. Such intermediate result can be combined with additional information to assist a doctor, nurse, or other practitioner to diagnose that a subject suffers from the disease. Alternatively, the present invention can be used to detect cancerous cells in a subject-derived tissue, and provide a doctor with useful information to diagnose that the subject suffers from the disease.

For example, according to the present invention, when there is doubt regarding the presence of cancer cells in the tissue obtained from a subject, clinical decisions can be reached by considering the expression level of the WHSC1 gene and/or WHSC1L1 gene, plus a different aspect of the disease including tissue pathology, levels of known tumor marker(s) in blood, and clinical course of the subject, etc. For example, some well-known diagnostic cancer markers in blood include ACT, AFP, BCA225, BFP, CA15-3, CA19-9, CA50, CA72-4, CA125, CA130, CA602, CEA, DUPAN-2, IAP, KMO-1, alpha-macrogloblin, NCC-ST-439, NSE, PIVKA-II, SCC, sICAM-1, SLX, SP1, SOD, Span-1, STN, TK activity, TPA, YH-206, elastase I, cytokeratin-19 fragment, and CYFRA21-1. Namely, in this particular embodiment of the present invention, the outcome of the gene expression analysis serves as an intermediate result for further diagnosis of a subject's disease state.

Specifically, the present invention provides the following methods [1] to [10]:

[1] A method of detecting or diagnosing cancer in a subject, including determining an expression level of a WHSC1 gene and/or an expression level of a WHSC1L1 gene in a subject-derived biological sample, wherein an increase of the expression level compared to a normal control level of the gene indicates that the subject suffers from or is at risk of developing cancer;

[2] A method of detecting cancer cells in a subject-derived biological sample, including the step of determining the expression level of the WHSC1 and/or WHSC1L1 gene in the sample, wherein an increase in the expression level as compared to a normal control level of the gene indicates the presence or suspicion of cancer cells in the sample;

[3] The method of [1] or [2], wherein the expression level is at least 10% greater than the normal control level;

[4] The method of any one of [1] to [3], wherein the expression level is detected by a method selected from among:

(a) detecting an mRNA of WHSC1 and/or an mRNA of WHSC1L1;

(b) detecting a protein encoded by a WHSC1 gene and/or a protein encoded by a WHSC1L1 gene; and

(c) detecting a biological activity of a protein encoded by a WHSC1 gene and/or a biological activity of a protein encoded by a WHSC1L1 gene;

[5] The method of any one of [1] to [4], wherein the cancer is bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma;

[6] The method of any one of [1] to [5], wherein the expression level is determined by detecting the hybridization level between a probe directed against the mRNA and the mRNA;

[7] The method of any one of [1] to [5], wherein the expression level is determined by detecting the binding level between an antibody against the protein and the protein;

[8] The method of any one of [1] to [7], wherein the biological sample includes a biopsy sample or specimen, urine, sputum or blood;

[9] The method of any one of [1] to [8], wherein the subject-derived biological sample includes an epithelial cell;

[10] The method of any one of [1] to [9], wherein the subject-derived biological sample includes a cancer cell;

[11] The method of any one of [1] to [10], wherein the subject-derived biological sample includes a cancerous epithelial cell; and

[12] The method of any one of [1] to [11], wherein the subject-derived biological sample includes cells derived from bladder, breast, biliary tract, bone marrow, esophagus, liver, lung, bone, pancreas, prostate, kidney, soft tissue or lymph node.

In another embodiment, [1] a method of detecting or diagnosing cancer in a subject, including determining either of an expression level of a WHSC1 gene or an expression level of a WHSC1L1 gene, or both in a subject-derived biological sample, wherein an increase of the expression level compared to a normal control level of the gene indicates that the subject suffers from or is at risk of developing cancer is provided.

In addition, in another embodiment, [2] a method of detecting cancer cells in a subject-derived biological sample, including the step of determining either of the expression level of the WHSC1 or WHSC1L1 gene, or both in the sample, wherein an increase in the expression level as compared to a normal control level of the gene indicates the presence or suspicion of cancer cells in the sample is also provided.

Alternatively, in another embodiment, [3] the method of any one of [1] to [3], wherein the expression level is detected by a method selected from among:

(a) detecting either of an mRNA of WHSC1 or an mRNA of WHSC1L1, or both;

(b) detecting a protein encoded by either of a WHSC1 gene or a protein encoded by a WHSC1L1 gene, or both, and;

(c) detecting a biological activity of a protein encoded by either of a WHSC1 gene or a biological activity of a protein encoded by a WHSC1L1 gene, or both is also provided. The method of diagnosing cancer or detecting cancer cells will be described in more detail below.

A subject to be diagnosed or from whom a biological sample is obtained by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the diagnosis of cancer or detection of cancer cells so long as it includes the transcription or translation product of WHSC1 and/or WHSC1L1. The biological samples include, but are not limited to, bodily tissues which are desired for diagnosing or are suspicion of suffering from cancer such as a biopsy specimen or sample, and fluids, such as blood, sputum and urine. Preferably, the biological sample contains a cell population including an epithelial cell, more preferably a cancerous epithelial cell or an epithelial cell derived from tissue suspected to be cancerous. Further, if necessary, a cell population can be purified from a obtained bodily tissue and fluid, and then used as a biological sample.

In preferred embodiments, cancers to be diagnosed include, but are not limited to, bladder cancer, breast cancer, cholangio cellular carcinoma, CML, esophageal cancer, HCC, NSCLC, SCLC, osteosarcoma, pancreatic cancer, prostate cancer, renal cell carcinoma, soft tissue tumor and lymphoma. Also, cancer cells to be detected, include, but are not limited to, bladder cancer cells, breast cancer cells, cholangio cellular carcinoma cells, CML cells, esophageal cancer cells, HCC cells, NSCLC cells, SCLC cells, osteosarcoma cells, pancreatic cancer, prostate cancer, renal cell carcinoma, soft tissue tumor cells and lymphoma cells. In order to diagnose such cancers or detecting such cancer cells, biological samples derived from following organs collected from a subject can be used as biological samples:

bladder for bladder cancer;

breast for breast cancer;

biliary tract for cholangiocellular carcinoma;

lymphocyte, blood sample including lymphocyte, bone marrow for CML;

esophagus for esophageal cancer;

liver for HCC;

lung for NSCLC and SCLC;

bone for osteosarcoma;

pancreas for pancreatic cancer;

prostate for prostate cancer;

kidney for renal cell carcinoma;

soft tissue for soft tissue tumor; and

lymphocyte or lymph node for lymphoma.

Preferably, biological samples can be collected from sites suspected to be cancerous in aforementioned organs. Therefore, a biopsy tissue, or a surgically resected tissue collected from bladder, breast, biliary tract, lymphocyte, bone marrow, esophagus, liver, lung, bone, pancreas, prostate, kidney, soft tissue, lymphocyte or lymph node, is preferable as a biological sample of the present invention.

According to the present invention, the expression level of WHSC1 and/or WHSC1L1 in the subject-derived biological sample is determined. The expression level can be determined at the transcription (nucleic acid) product level, using methods known in the art. For example, the mRNA of WHSC1 and/or WHSC1L1 can be quantified using probes by hybridization methods (e.g., Northern hybridization). The detection can be carried out on a chip or an array. The use of an array is preferable for detecting the expression level of a plurality of genes (e.g., various cancer specific genes) including WHSC1 and/or WHSC1L1. Those skilled in the art can prepare such probes utilizing the sequence information of WHSC1 and/or WHSC1L1. For example, the cDNA of WHSC1 or WHSC1L1 can be used as the probes. If necessary, the probe can be labeled with a suitable label, such as dyes, fluorescent and isotopes, and the expression level of the gene can be detected as the intensity of the hybridized labels.

Furthermore, the transcription product of WHSC1 and/or WHSC1L1 can be quantified using primers by amplification-based detection methods (e.g., RT-PCR). Such primers can also be prepared based on the available sequence information of the gene. For example, the primers (SEQ ID NOs: 9 and 10 or 11 and 12 for WHSC1, and 13 and 14 or 15 and 16 for WHSC1L1) used in the Example can be employed for the detection by RT-PCR or Northern blot, but the present invention is not restricted thereto.

Specifically, a probe or primer used for the present method hybridizes under stringent, moderately stringent, or low stringent conditions to the mRNA of WHSC1 or WHSC1L1. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions can also be achieved with the addition of destabilizing agents, such as formamide.

Alternatively, the translation product can be detected for the diagnosis of the present invention. For example, the quantity of WHSC1 and/or WHSC1L1 protein can be determined. A method for determining the quantity of the protein as the translation product includes immunoassay methods that use an antibody specifically recognizing the protein. The antibody can be monoclonal or polyclonal. Furthermore, any fragment or modification (e.g., chimeric antibody, scFv, Fab, F(ab′)2, Fv, etc.) of the antibody can be used for the detection, so long as the fragment retains the binding ability to WHSC1 or WHSC1L1 protein. Methods to prepare these kinds of antibodies for the detection of proteins are well known in the art, and any method can be employed in the present invention to prepare such antibodies and equivalents thereof.

As another method to detect the expression level of WHSC1 and/or WHSC1L1 gene based on its translation product, the intensity of staining can be observed via immunohistochemical analysis using an antibody against WHSC1 or WHSC1L1 protein. Namely, the observation of strong staining indicates increased presence of the protein and at the same time high expression level of the gene.

Moreover, in addition to the expression level of WHSC1 and/or WHSC1L1 gene, the expression level of other cancer-associated genes, for example, genes known to be differentially expressed in cancer can also be determined to improve the accuracy of the diagnosis.

The expression level of cancer marker gene including WHSC1 and WHSC1L1 gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level can be determined at the same time with the test biological sample by using a sample(s) previously collected and stored from a subject/subjects whose disease state (cancerous or non-cancerous) is/are known. Alternatively, the control level can be determined by a statistical method based on the results obtained by analyzing previously determined expression level(s) of WHSC1 or WHSC1L1 gene in samples from subjects whose disease state are known. Furthermore, the control level can be a database of expression patterns from previously tested cells. Moreover, according to an aspect of the present invention, the expression level of WHSC1 or WHSC1L1 gene in a biological sample can be compared to multiple control levels, which control levels are determined from multiple reference samples. It is preferred to use a control level determined from a reference sample derived from a tissue type similar to that of the subject-derived biological sample. Moreover, it is preferred to use the standard value of the expression levels of WHSC1 or WHSC1L1 gene in a population with a known disease state. The standard value can be obtained by any method known in the art. For example, a range of mean+/−2 S.D. or mean+/−3 S.D. can be used as standard value.

In the context of the present invention, a control level determined from a biological sample that is known not to be cancerous is referred to as a “normal control level”. On the other hand, if the control level is determined from a cancerous biological sample, it is referred to as a “cancerous control level”.

When the expression level of WHSC1 and/or WHSC1L1 gene is increased as compared to the normal control level or is similar to the cancerous control level, the subject can be diagnosed to be suffering from or at a risk of developing cancer. Furthermore, in the case where the expression levels of multiple cancer-related genes are compared, a similarity in the gene expression pattern between the sample and the reference which is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

In another aspect, the present invention provides a method of identifying a subject suspected of suffering from cancer, including the step of determining an expression level of WHSC1 gene and/or an expression level of WHSC1L1 gene in a subject-derived biological sample, wherein an increase of the level compared to a normal control level of the gene indicates that the subject is suffering from cancer, wherein the expression level is determined by a method selected from the group consisting of:

(a) detecting an mRNA of WHSC1 or an mRNA of WHSC1L1;

(b) detecting a protein encoded by the WHSC1 gene or a protein encoded by the WHSC1L1 gene; and

(c) detecting a biological activity of a protein encoded by the WHSC1 gene or a biological activity of a protein encoded by the WHSC1L1 gene.

In another aspect, the present invention provides a method of identifying a subject-derived biological sample suspected of containing cancer cells, including the step of determining an expression level of WHSC1 gene and/or an expression level of WHSC1L1 gene in the biological sample, wherein an increase of the level compared to a normal control level of the gene indicates that the biological sample is suspected to contain cancer cells, wherein the expression level is determined by a method selected from the group consisting of:

(a) detecting an mRNA of WHSC1 or an mRNA of WHSC1L1;

(b) detecting a protein encoded by WHSC1 gene or a protein encoded by WHSC1L1 gene; and

(c) detecting a biological activity of a protein encoded by WHSC1 gene or a biological activity of a protein encoded by WHSC1L1 gene.

Determining an expression level of WHSC1 gene or WHSC1L1 gene can be conducted by the methods described above. After identifying a candidate subject or a suspicious biological sample, such candidate subject or sample can be further examined, for example, by other tumor markers, imaging analysis, pathological observation, and so on.

A Kit for Diagnosing Cancer:

The present invention provides a kit for diagnosing cancer. Preferably, the cancer is bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma. Specifically, the kit includes at least one reagent for detecting an expression level of the WHSC1 gene and/or WHSC1L1 gene in a subject-derived biological sample, which reagent can be selected from the group consisting of:

(a) a reagent for detecting an mRNA of the WHSC1 gene and/or an mRNA of the WHSC1L1 gene;

(b) a reagent for detecting a protein encoded by the WHSC1 gene and/or a protein encoded by the WHSC1L1 gene; and

(c) a reagent for detecting a biological activity of a protein encoded by the WHSC1 gene and/or a biological activity of a protein encoded by the WHSC1L1 gene.

Alternatively, the kit includes at least one reagent for detecting either of an expression level of the WHSC1 gene or an expression level of the WHSC1L1 gene, or both in a subject-derived biological sample, which reagent can be selected from the group consisting of:

(a) a reagent for detecting either of an mRNA of the WHSC1 gene or an mRNA of the WHSC1L1 gene, or both;

(b) a reagent for detecting either of a protein encoded by the WHSC1 gene or a protein encoded by the WHSC1L1 gene, or both; and

(c) a reagent for detecting either of a biological activity of a protein encoded by the WHSC1 gene or a biological activity of a protein encoded by the WHSC1L1 gene, or both.

Suitable reagents for detecting mRNA of the WHSC1 or WHSC1L1 gene include nucleic acids that specifically bind to or identify the WHSC1 mRNA or WHSC1L1 mRNA, such as oligonucleotides which have a complementary sequence to a part of the WHSC1 mRNA or WHSC1L1 mRNA. These kinds of oligonucleotides are exemplified by primers and probes that are specific to the WHSC1 mRNA or WHSC1L1 mRNA. These kinds of oligonucleotides can be prepared based on methods well known in the art. If needed, the reagent for detecting the WHSC1 mRNA or WHSC1L1 mRNA can be immobilized on a solid matrix. Moreover, more than one reagent for detecting the WHSC1 mRNA or WHSC1L1 mRNA can be included in the kit.

A probe or primer of the present invention typically comprises a substantially purified oligonucleotide. The oligonucleotide typically comprises a region of nucleotide sequence that hybridizes under stringent conditions to at least about 2000, 1000, 500, 400, 350, 300, 250, 200, 150, 100, 50, or 25, consecutive sense strand nucleotide sequence of a nucleic acid comprising a WHSC1 or WHSC1L1 sequence, or an antisense strand nucleotide sequence of a nucleic acid comprising a WHSC1 or WHSC1L1 sequence, or of a naturally occurring mutant of these sequences. In particular, for example, in a preferred embodiment, an oligonucleotide having 5-50 in length can be used as a primer for amplifying the genes, to be detected. More preferably, mRNA or cDNA of WHSC1 or WHSC1L1 gene can be detected with oligonucleotide probe or primer having 15-30b in length. In preferred embodiments, length of the oligonucleotide probe or primer can be selected from 15-25. Assay procedures, devices, or reagents for the detection of gene by using such oligonucleotide probe or primer are well known (e.g. oligonucleotide microarray or PCR). In these assays, probes or primers can also comprise tag or linker sequences. Further, probes or primers can be modified with detectable label or affinity ligand to be captured. Alternatively, in hybridization based detection procedures, a polynucleotide having a few hundreds (e.g., about 100-200) bases to a few kilo (e.g., about 1000-2000) bases in length can also be used for a probe (e.g., northern blotting assay or cDNA microarray analysis). In some embodiments, a nucleotide sequence of the probe or primer can be selected from regions specific for each transcript of WHSC1 or WHSC1L1. Alternatively, a probe or primer which recognizes both of transcripts of WHSC1 and WHSC1L1 is also suitable for the detection of both of the transcripts.

In the present invention, it is revealed that WHSC1 or WHSC1L1 is not only a useful diagnostic marker, but also suitable target for cancer therapy. Therefore, cancer treatment targeting WHSC1 or WHSC1L1 can be achieved by the present invention. In the present invention, the cancer treatment targeting WHSC1 or WHSC1L1 refers to suppression or inhibition of either or both of WHSC1 activity and expression, or either or both of WHSC1L1 activity and expression in the cancer cells. Any anti-WHSC1 or anti-WHSC1L1 agents may be used for the cancer treatment targeting WHSC1 or WHSC1L1. In the present invention, the anti-WHSC1 or anti-WHSC1L1 agents include following substances as active ingredient:

(a) a double-stranded molecule of the present invention,
(b) DNA encoding said double-stranded molecule, or
(c) a vector encoding said double-stranded molecule.

Additional suitable reagents can be reagents for detecting for detecting the WHSC1 protein or WHSC1L1 protein. Such reagents include antibodies to the WHSC1 protein or WHSC1L1 protein. The antibody can be monoclonal or polyclonal. Furthermore, any fragment or modification (e.g., chimeric antibody, scFv, Fab, F(ab′)2, Fv, etc.) of the antibody can be used as the reagent, so long as the fragment retains the binding ability to the WHSC1 protein or WHSC1L1 protein. Methods to prepare these kinds of antibodies for the detection of the protein are well known in the art, and any method can be employed in the present invention to prepare such antibodies and equivalents thereof. Furthermore, the antibody can be labeled with signal generating molecules via direct linkage or an indirect labeling technique. Labels and methods for labeling antibodies and detecting the binding of antibodies to their targets are well known in the art and any labels and methods can be employed for the present invention. Moreover, more than one reagent for detecting the WHSC1 protein or WHSC1L1 protein can be included in the kit.

Furthermore, the biological activity can be determined by, for example, measuring the cell proliferating activity due to the expressed WHSC1 and/or WHSC1L1 protein in the biological sample. For example, a cell is cultured in the presence of a subject-derived biological sample, and then by detecting the speed of proliferation, or by measuring the cell cycle or the colony forming ability, the cell proliferating activity of the biological sample can be determined. Moreover, more than one reagent for detecting the biological activity of the WHSC1 protein or WHSC1L1 protein can be included in the kit.

The kit may contain more than one of the aforementioned reagents. Furthermore, the kit may include a solid matrix and reagent for binding a probe directed against the WHSC1 gene or WHSC1L1 gene or antibody against the proteins, a medium and container for culturing cells, positive and negative control reagents, and a secondary antibody for detecting an antibody against the WHSC1 protein or WHSC1L1 protein. For example, tissue samples obtained from a subject suffering from cancer or a control subject not suffering from cancer can serve as useful control reagents. A kit of the present invention can further include other materials desirable from a commercial and user standpoint, including buffers, diluents, filters, needles, syringes, and package inserts (e.g., written, tape, CD-ROM, etc.) with instructions for use. These reagents and such may be included in a container with a label. Suitable containers include bottles, vials, and test tubes. The containers can be formed from a variety of materials, such as glass or plastic.

As an embodiment of the present invention, when the reagent is a probe directed against the WHSC1 mRNA or WHSC1L1 mRNA, the reagent can be immobilized on a solid matrix, such as a porous strip, to form at least one detection site. The measurement or detection region of the porous strip may include a plurality of sites, each containing a nucleic acid (probe). A test strip can also contain sites for negative and/or positive controls. Alternatively, control sites can be located on a strip separated from the test strip. Optionally, the different detection sites can contain different amounts of immobilized nucleic acids, i.e., a higher amount in the first detection site and lesser amounts in subsequent sites. Upon the addition of test sample, the number of sites displaying a detectable signal provides a quantitative indication of the amount of WHSC1 mRNA or WHSC1L1 mRNA present in the sample. The detection sites can be configured in any suitably detectable shape and are typically in the shape of a bar or dot spanning the width of a test strip.

The kit of the present invention can further include a positive control sample or WHSC1 standard sample and/or WHSC1L1 standard sample. The positive control sample of the present invention can be prepared by collecting WHSC1 and/or WHSC1L1 positive samples and then assaying the WHSC1 and/or WHSC1L1 levels. In one embodiment, the WHSC1 or WHSC1L1 positive tissue samples can be composed of cancer cells expressing WHSC1 or WHSC1L1. Such cancer includes, but are not limited to, bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma.

The WHSC1 level and/or WHSC1L1 level of the positive control sample can be, for example, more than cut off value. For example, positive control samples can be prepared by determined a cut-off value and preparing a sample containing an amount of the WHSC1 mRNA or protein and/or an amount of the WHSC1L1 mRNA or protein more than the cut-off value. Herein, the phrase “cut-off value” refers to the value dividing between a normal range and a cancerous range. For example, one skilled in the art can be determine a cut-off value using a receiver operating characteristic (ROC) curve.

Alternatively, the present kit can include a WHSC1 standard sample containing a cut-off value amount of a WHSC1 mRNA or protein and/or WHSC1L1 standard sample containing a cut-off value amount of a WHSC1L1 mRNA or protein.

Alternatively, the present kit includes a negative control sample. The negative control sample can be prepared from non-cancerous cell lines or non-cancerous tissues, or can be prepared by preparing a sample containing a WHSC1 mRNA or protein less than cut-off value and/or a WHSC1L1 mRNA or protein less than cut-off value.

In another aspect, the present invention provides a probe or a primer set directed against WHSC1 mRNA or WHSC1L1 mRNA, or an antibody against WHSC1 protein or WHSC1L1 protein for use in diagnosis of cancer.

In another aspect, the present invention provide a reagent for diagnosing cancer in a subject, comprising a probe or a primer set directed against WHSC1 mRNA or WHSC1L1 mRNA, or an antibody against WHSC1 protein or WHSC1L1 protein.

In another aspect, the present invention provides use of a probe or a primer set directed against WHSC1 mRNA or WHSC1L1 mRNA, or an antibody against WHSC1 protein or WHSC1L1 protein for the manufacture of a reagent for diagnosis of cancer.

Screening for an Anti-Cancer Substance:

In the context of the present invention, substances to be identified through the present screening methods can be any compounds or compositions including several compounds. Furthermore, the test substance exposed to a cell or protein according to the screening methods of the present invention can be a single compound or a combination of compounds. When a combination of compounds is used in the methods, the compounds can be contacted sequentially or simultaneously.

Any test substances, for example, cell extracts, cell culture supernatants, products of fermenting microorganism, extracts from a marine organism, plant extracts, purified or crude proteins, peptides, non-peptide substances, synthetic micromolecular substances (including nucleic acid constructs, such as antisense RNA, siRNA, Ribozymes, and aptamer etc.) and natural substances can be used in the screening methods of the present invention. The test substance of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds can be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A substance in which a part of the structure is converted by addition, deletion and/or replacement, is included in the substance obtained by the screening methods of the present invention.

Furthermore, when the screened test substance is a protein, for obtaining a DNA encoding the protein, either the whole amino acid sequence of the protein can be determined to deduce the nucleic acid sequence coding for the protein, or partial amino acid sequence of the obtained protein can be analyzed to prepare an oligo DNA as a probe based on the sequence, and screen cDNA libraries with the probe to obtain a DNA encoding the protein. The obtained DNA is confirmed its usefulness in preparing the test substance which is a candidate for treating or preventing cancer.

Test substances useful in the screenings described herein can also be antibodies that specifically bind to WHSC1 protein or WHSC1L1 protein or partial peptides of WHSC1 protein or WHSC1L1 protein that lack the biological activity of the original proteins in vivo.

It is herein revealed that suppression of either or both of the expression level and biological activity of WHSC1 or WHSC1L1 lead to suppression of the growth of cancer cells. Therefore, when a substance suppresses either or both of the expression and activity of WHSC1 or WHSC1L1, such suppression is indicative of a potential therapeutic effect in a subject. In the context of the present invention, a potential therapeutic effect refers to a clinical benefit with a reasonable expectation. Examples of such clinical benefit include but are not limited to;

(a) reduction in expression of the WHSC1 or WHSC1L1 gene,

(b) a decrease in size, prevalence, or metastatic potential of cancer in a subject,

(c) preventing cancer from forming, or

(d) preventing or alleviating a clinical symptom of cancer.

Although the construction of test substance libraries is well known in the art, additional guidance in identifying test substances and construction libraries of such substances for the present screening methods are provided below.

(i) Molecular Modeling:

Construction of test substance libraries is facilitated by knowledge of the molecular structure of compounds known to have the properties sought, and/or the molecular structure of WHSC1 protein or WHSC1L1 protein. One approach to preliminary screening of test substances suitable for further evaluation is computer modeling of the interaction between the test substance and its target.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test substances” can be screened using the methods of the present invention to identify candidate substances for treating or preventing cancer, such as bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma.

(ii) Combinatorial Chemical Synthesis:

Combinatorial libraries of test substances can be produced as part of a rational drug design program involving knowledge of core structures existing in known inhibitors. This approach allows the library to be maintained at a reasonable size, facilitating high throughput screening. Alternatively, simple, particularly short, polymeric molecular libraries can be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and can be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

(iii) Other Candidates:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules {e.g., 10¹⁵ different molecules) can be used for screening.

(1) Polypeptide Based Screening:

The present invention provides methods of screening for a candidate substance applicable to the treatment and/or prevention of cancer using a WHSC1 or WHSC1L1 polypeptide.

In the context of the present screening method, the WHSC1 or WHSC1L1 polypeptide to be used can be, for example, a purified polypeptide, a soluble protein, a form bound to a carrier or a fusion protein fused with other polypeptides. Further, the WHSC1 or WHSC1L1 polypeptide can be a recombinant polypeptide, a protein derived from the nature or a partial peptide thereof.

In addition to naturally-occurring WHSC1 or WHSC1L1 polypeptides, functional equivalents of the polypeptides can be included in WHSC1 or WHSC1L1 polypeptides used for the present screening so long as the modified peptide retains at least one biological activity of the original polypeptide. Examples of the biological activity of the WHSC1 or WHSC1L1 polypeptide include, but are not limited to, cell proliferative activity, methyltransferase activity, binding activity to IQGAP1 polypeptide, TIAM1 polypeptide, AKT2 polypeptide or beta-catenin polypeptide. Preferred examples of such functional equivalents are described above in the section entitled “Genes and proteins of WHSC1 and WHSC1L1”. For example, a preferred example of such functional equivalents includes a polypeptide containing the SET domain of WHSC1 polypeptide (e.g., 1066-1179 of SEQ ID NO: 2). Also, preferred examples of functional equivalents of WHSC1L1 protein include polypeptides containing the SET domain of WHSC1L1 polypeptide (e.g., 1148 to 1261 of SEQ ID NO: 50).

The polypeptides can be further linked to other substances, so long as the linking process and linked substance do not interfere with the biological activity of the original polypeptide and/or fragment. Usable substances include, for example: peptides, lipids, sugar and sugar chains, acetyl groups, natural and synthetic polymers, etc. These kinds of modifications can be performed to confer additional functions or to stabilize the polypeptide and fragments. The polypeptides used for the present method can be obtained from nature as naturally occurring proteins via conventional purification methods or through chemical synthesis based on a selected amino acid sequence. For example, conventional peptide synthesis methods that can be adopted for the synthesis include:
1) Peptide Synthesis, Interscience, New York, 1966;
2) The Proteins, Vol. 2, Academic Press, New York, 1976;
3) Peptide Synthesis (in Japanese), Maruzen Co., 1975;
4) Basics and Experiment of Peptide Synthesis (in Japanese), Maruzen Co., 1985;
5) Development of Pharmaceuticals (second volume) (in Japanese), Vol. 14 (peptide synthesis), Hirokawa, 1991;
6) WO99/67288; and
7) Barany G. & Merrifield R. B., Peptides Vol. 2, “Solid Phase Peptide Synthesis”, Academic Press, New York, 1980, 100-118.

Alternatively, the polypeptides can be obtained by adapting any known genetic engineering methods to the production of the instant polypeptides (e.g., Morrison J., J Bacteriology 1977, 132: 349-51; Clark-Curtiss & Curtiss, Methods in Enzymology (eds. Wu et al.) 1983, 101: 347-62). For example, first, a suitable vector including a polynucleotide encoding the objective protein in an expressible form (e.g., downstream of a regulatory sequence including a promoter) is prepared, transformed into a suitable host cell, and then the host cell is cultured to produce the protein. More specifically, a gene encoding a WHSC1 or WHSC1L1 polypeptide are expressed in host (e.g., animal) cells and such by inserting the gene into a vector for expressing foreign genes, such as pSV2neo, pcDNA I, pcDNA3.1, pCAGGS, or pCD8. A promoter can be used for the expression. Any commonly used promoters can be employed, including, for example, the SV40 early promoter (Rigby in Williamson (ed.), Genetic Engineering, vol. 3. Academic Press, London, 1982, 83-141), the EF-alpha promoter (Kim et al., Gene 1990, 91:217-23), the CAG promoter (Niwa et al., Gene 1991, 108:193), the RSV LTR promoter (Cullen, Methods in Enzymology 1987, 152:684-704), the SR-alpha promoter (Takebe et al., Mol Cell Biol 1988, 8:466), the CMV immediate early promoter (Seed et al., Proc Natl Acad Sci USA 1987, 84:3365-9), the SV40 late promoter (Gheysen et al., J Mol Appl Genet. 1982, 1:385-94), the Adenovirus late promoter (Kaufman et al., Mol Cell Biol 1989, 9:946), the HSV TK promoter, and such. The introduction of the vector into host cells to express an WHSC10R WHSC1L1 polypeptide can be performed according to any conventional methods, for example, the electroporation method (Chu et al., Nucleic Acids Res 1987, 15:1311-26), the calcium phosphate method (Chen et al., Mol Cell Biol 1987, 7:2745-52), the DEAE dextran method (Lopata et al., Nucleic Acids Res 1984, 12:5707-17; Sussman et al., Mol Cell Biol 1985, 4:1641-3), the Lipofectin method (Derijard B, Cell 1994, 7:1025-37; Lamb et al., Nature Genetics 1993, 5:22-30; Rabindran et al., Science 1993, 259:230-4), and such.

WHSC1 or WHSC1L1 polypeptides can also be produced in vitro using a conventional in vitro translation system.

(i) Screening for a Substance Binding to a WHSC1 or WHSC1L1 Polypeptide:

In the present invention, over-expression of WHSC1 gene was detected in bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor, in spite of little expression in normal organs (FIG. 1, 2, 3 and Table 5). Further, over-expression of WHSC1L1 gene was detected in bladder cancer, breast cancer, CML, lung cancer (e.g., SCLC) and lymphoma in spite of little expression in normal organs (FIG. 1 and Table 5). Therefore, using the WHSC1 and/or WHSC1L1 genes and polypeptides encoded by the genes, the present invention provides a method of screening for a substance that binds to WHSC1 polypeptide or WHSC1L1 polypeptide. Due to the expression of WHSC1 gene and WHSC1L1 gene in cancer, a substance binds to WHSC1 polypeptide or WHSC1L1 polypeptide is expected to suppress the proliferation of cancer cells, and thus be useful for treating or preventing cancer. Therefore, the present invention also provides a method for screening a candidate substance that suppresses the proliferation of cancer cells, and a method for screening a candidate substance for treating or preventing cancer using the WHSC1 polypeptide or WHSC1L1 polypeptide. Specially, an embodiment of this screening method includes the steps of:

(a) contacting a test substance with a polypeptide encoded by a WHSC1 or WHSC1L1 gene;

(b) detecting the binding activity between the polypeptide and the test substance; and

(c) selecting the test substance that binds to the polypeptide.

Alternatively, according to the present invention, the potential therapeutic effect of a test substance or compound on treating or preventing cancer can also be evaluated or estimated. In some embodiments, the present invention provides a method for evaluating or estimating a therapeutic effect of a test substance on treating or preventing cancer associated with over-expression of WHSC1 or WHSC1L1, the method including steps of:

(a) contacting a test substance with a polypeptide encoded by a polynucleotide of WHSC1 or WHSC1L1;

(b) detecting the binding activity between the polypeptide and the test substance; and

(c) correlating the potential therapeutic effect and the test substance, wherein the potential therapeutic effect is shown when a substance binds to the polypeptide.

The method of the present invention will be described in more detail below.

The WHSC1 polypeptide (i.e., the polypeptide encoded by a WHSC1 gene) or WHSC1L1 polypeptide (i.e., the polypeptide encoded by a WHSC1L1 gene) to be used for screening can be a recombinant polypeptide or a protein derived from the nature or a partial peptide thereof. The polypeptide to be contacted with a test substance can be, for example, a purified polypeptide, a soluble protein, a form bound to a carrier or a fusion protein fused with other polypeptides.

As a method of screening for proteins, for example, that bind to the WHSC1 or WHSC1L1 polypeptide using the WHSC1 or WHSC1L1 polypeptide, many methods well known by a person skilled in the art can be used. Such a screening can be conducted by, for example, immunoprecipitation method, specifically, in the following manner. The gene encoding the WHSC1 or WHSC1L1 polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression can be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet. 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by WHSC1 or WHSC1L1 gene can be expressed as a fusion protein including a recognition site (i.e., epitope) of a monoclonal antibody whose specificity has been revealed by introducing such epitope to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the polypeptide by the fusion can be used. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the WHSC1 or WHSC1L1 polypeptide (Experimental Medicine 13: 85-90 (1995)).

In immunoprecipitation, an immune complex is formed by adding these antibodies to cell lysate prepared using an appropriate detergent. The immune complex consists of the WHSC1 or WHSC1L1 polypeptide, a polypeptide including the binding ability with the polypeptide, and an antibody. Immunoprecipitation can be also conducted using antibodies against the WHSC1 or WHSC1L1 polypeptide, besides using antibodies against the above epitopes, which antibodies can be prepared as described above. An immune complex can be precipitated, for example by Protein A sepharose or Protein G sepharose when the antibody is a mouse IgG antibody. If the polypeptide encoded by WHSC1 or WHSC1L1 gene is prepared as a fusion protein with an epitope, such as GST, an immune complex can be formed in the same manner as in the use of the antibody against the WHSC1 or WHSC1L1 polypeptide, using a substance specifically binding to these epitopes, such as glutathione-Sepharose 4B. Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the WHSC1 or WHSC1L1 polypeptide is difficult to detect by a common staining method, such as Coomassie staining or silver staining, the detection sensitivity for the protein can be improved by culturing cells in culture medium containing radioactive isotope, “S-methionine or” S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to the WHSC1 or WHSC1L1 polypeptide using the polypeptide, for example, West-Western blotting analysis (Skolnik et al., Cell 65: 83-90 (1991)) can be used. Specifically, a protein binding to the WHSC1 or WHSC1L1 polypeptide can be obtained by preparing a cDNA library from cultured cells expected to express a protein binding to the WHSC1 or WHSC1L1 polypeptide using a phage vector (e.g., ZAP), expressing the protein on LB-agarose, fixing the protein expressed on a filter, reacting the purified and labeled WHSC1 or WHSC1L1 polypeptide with the above filter, and detecting the plaques expressing proteins bound to the WHSC1 or WHSC1L1 polypeptide according to the label. The WHSC1 or WHSC1L1 polypeptide can be labeled by utilizing the binding between biotin and avidin, or by utilizing an antibody that specifically binds to the WHSC1 or WHSC1L1 polypeptide, or a peptide or polypeptide (for example, GST) that is fused to the WHSC1 or WHSC1L1 polypeptide. Methods using radioisotope or fluorescence and such can be also used.

Alternatively, in another embodiment of the screening method of the present invention, a two-hybrid system utilizing cells can be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, the WHSC1 or WHSC1L1 polypeptide is fused to the SRF-binding region or GAL4-binding region and expressed in yeast cells. A cDNA library is prepared from cells expected to express a protein binding to the WHSC1 or WHSC1L1 polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A substance binding to the polypeptide encoded by WHSC1 or WHSC1L1 gene can also be screened using affinity chromatography. For example, the WHSC1 or WHSC1L1 polypeptide can be immobilized on a carrier of an affinity column, and a test substance, containing a protein capable of binding to the polypeptide of the invention, is applied to the column. A test substance herein can be, for example, cell extracts, cell lysates, etc. After loading the test substance, the column is washed, and substances bound to the polypeptide of the invention can be prepared. When the test substance is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon can be used as a means for detecting or quantifying the bound substance in the present invention. When such a biosensor is used, the interaction between the WHSC1 or WHSC1L1 polypeptide and a test substance can be observed real-time as a surface plasmon resonance signal, using only a minute amount of polypeptide and without labeling (for example, BIAcore, Pharmacia). Therefore, it is possible to evaluate the binding between the polypeptide of the invention and a test substance using a biosensor such as BIAcore.

The methods of screening for molecules that bind when the immobilized WHSC1 or WHSC1L1 polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the WHSC1 or WHSC1L1 protein (including agonist and antagonist) are well known to one skilled in the art.

(ii) Screening for a Substance Suppressing the Biological Activity of WHSC1 or WHSC1L1 Polypeptide:

In the present invention, the WHSC1 and WHSC1L1 polypeptides have the activity of promoting cell proliferation of cancer cells (FIG. 4). Moreover, the WHSC1 and WHSC1L1 polypeptides are known to have histone methyltransferase activity. As it has been demonstrated herein that WHSC1 and WHSC1L1 polypeptides play crucial roles in cancer cell survival, substances that suppress those biological activities of WHSC1 or WHSC1L1 polypeptide can be candidate drugs for cancer therapy. Therefore, the present invention provides a method for screening a substance that suppresses the proliferation of cancer cells expressing WHSC1 and/or WHSC1L1, and a method for screening a candidate substance for treating or preventing cancer, using above-mentioned biological activities as an index. Substances screened by the method of the present invention can be candidate drugs for any cancers as long as the cancers are associated with WHSC1 and/or WHC1L1 overexpression. For example, cancers associated with WHSC1 overexpression include, but are not limited to, bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor. Also, for example, cancer associated with WHSC1L1 overexpression include, but are not limited to, bladder cancer, breast cancer, CML, lung cancer (e.g., SCLC) and lymphoma.

Specifically, the present invention provides a method of screening for a candidate substance for treating and/or preventing cancer using the polypeptide encoded by WHSC1 or WHSC1L1 gene including the steps as follows:

(a) contacting a test substance with a polypeptide encoded by WHSC1 or WHSC1L1 gene;

(b) detecting a biological activity of the polypeptide of step (a); and

(c) selecting the test substance that suppresses the biological activity of the polypeptide as compared to the biological activity of the polypeptide detected in the absence of the test substance.

Alternatively, the present invention provides a method of screening for a candidate substance for either or both of treating and preventing cancer using the polypeptide encoded by WHSC1 or WHSC1L1 gene including the steps as follows:

(a) contacting a test substance with a polypeptide encoded by WHSC1 or WHSC1L1 gene;

(b) detecting a biological activity of the polypeptide of step (a); and

(c) selecting the test substance that suppresses the biological activity of the polypeptide as compared to the biological activity of the polypeptide detected in the absence of the test substance.

According to the present invention, the therapeutic effect of the test substance on suppressing the biological activity (e.g., the cell-proliferating activity or the methyltransferase activity) of WHSC1 or WHSC1L1 polypeptide, or a candidate substance for treating or preventing cancer can be evaluated. Therefore, the present invention also provides a method of screening for a candidate substance for suppressing the biological activity of WHSC1 or WHSC1L1 polypeptide, or a candidate substance for treating or preventing cancer, using the WHSC1 or WHSC1L1 polypeptide or fragments thereof, including the following steps:

(a) contacting a test substance with the WHSC1 or WHSC1L1 polypeptide or a functional fragment thereof; and
(b) detecting the biological activity of the polypeptide or fragment of step (a), and
(c) correlating the biological activity of (b) with the therapeutic effect of the test substance.

Alternatively, in some embodiments, the present invention provides a method for evaluating or estimating a therapeutic effect of a test substance on treating or preventing cancer associated with over-expression of WHSC1 or WHSC1L1, the method including steps of:

(a) contacting a test substance with a polypeptide encoded by a polynucleotide of WHSC1 or WHSC1L1 gene or a functional fragment thereof;

(b) detecting the biological activity of the polypeptide or the fragment of step (a); and

(c) correlating the potential therapeutic effect and the test substance, wherein the potential therapeutic effect is shown when a substance suppresses the biological activity of the polypeptide encoded by the polynucleotide of WHSC1 or WHSC1L1 gene or the fragment as compared to the biological activity of said polypeptide or the fragment detected in the absence of the test substance.

Such cancer includes bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor and lymphoma. In the present invention, the therapeutic effect can be correlated with the biological activity of the WHSC1 or WHSC1L1 polypeptide or a functional fragment thereof. For example, when the test substance suppresses or inhibits the biological activity of the WHSC1 or WHSC1L1 polypeptide or a functional fragment thereof as compared to a level detected in the absence of the test substance, the test substance can identified or selected as the candidate substance having the therapeutic effect. Alternatively, when the test substance does not suppress or inhibit the biological activity of the WHSC1 or WHSC1L1 polypeptide or a functional fragment thereof as compared to a level detected in the absence of the test substance, the test substance can identified as the substance having no significant therapeutic effect.

The method of the present invention will be described in more detail below.

Any polypeptides can be used for the screening method of the present invention so long as they have a biological activity of the WHSC1 or WHSC1L1 protein. Such biological activity includes, for example, cell-proliferating activity (cell proliferation promoting activity) and methyltransferase activity. For example, WHSC1 or WHSC1L1 protein can be used and polypeptides functionally equivalent to these proteins can also be used. Such polypeptides can be expressed endogenously or exogenously in cells.

The substance isolated by this screening method is a candidate for antagonists of the polypeptide encoded by WHSC1 or WHSC1L1 gene. The term “antagonist” refers to molecules that inhibit the function of the polypeptide by binding thereto. Said term also refers to molecules that reduce or inhibit expression of the gene encoding WHSC1 or WHSC1L1. Moreover, a substance isolated by this screening method is a candidate for substances which inhibit the in vivo interaction of the WHSC1 or WHSC1L1 polypeptide with molecules (including DNAs and proteins).

When the biological activity to be detected in the present screening method is cell proliferating activity, it can be detected, for example, by preparing cells which express the WHSC1 or WHSC1L1 polypeptide, culturing the cells in the presence of a test substance, and determining the speed of cell proliferation, measuring the cell cycle and such, as well as by measuring cell survival or the colony forming activity. The substances that reduce the speed of proliferation of the cells expressed WHSC1 or WHSC1L1 are selected as candidate substance for treating and/or preventing cancer.

More specifically, the method includes the step of:

(a) contacting a test substance with cells overexpressing WHSC1 or WHSC1L1;

(b) measuring cell-proliferating activity; and

(c) selecting the test substance that reduces the cell-proliferating activity in the comparison with the cell-proliferating activity detected in the absence of the test substance.

In preferable embodiments, the method of the present invention can further include the steps of:

(d) selecting the test substance that have no or little effect to the cells no or little expressing WHSC1 or WHSC1L1.

When the biological activity to be detected in the present screening method is methyltransferase activity, the methyltransferase activity can be determined by contacting WHSC1 or WHSC1L1 polypeptide with a substrate (e.g., histone H3 fragment including Lys-36 for WHSC1, histone H3 fragment including Lys-4 and/or Lys-27 for WHSC1L1 (Kim S M, et al. Biochem Biophys Res Commun. 2006 Jun. 23; 345(1):318-23)) and a co-factor (e.g., S-adenosyl-L-methionine) under conditions suitable for methylation of the substrate and detecting the methylation level of the substrate.

In the present invention, the screening methods using methyltransferase activity encompass the following methods of [1] to [7]:

[1] A method of screening for a candidate substance for treating or preventing cancer or inhibiting cancer cell growth, the method including the steps of:

(a) contacting a polypeptide encoded by WHSC1 or WHSC1L1 gene with a substrate and a cofactor in the presence of a test substance;

(b) detecting the methylation level of the substrate of step (a); and

(c) selecting the test substance that suppress the methylation level of the substrate as compared to the methylation level detected in the absence of the test substance;

[2] The method of [1], wherein the substrate is a histone or a fragment thereof including at least one methylation region for WHSC1 or WHSC1L1 polypeptide;

[3] The method of [2], wherein the substrate is a histone H3 or a fragment thereof including at least one methylation region for WHSC1 or WHSC1L1 polypeptide;

[4] The method of [3], wherein the methylation region is lysine 4, 27 or 36 of histone H3;

[5] The method of any one of [1] to [4], wherein the cofactor is an S-adenosylmethionine;

[6] The method of any one of [1] to [5], wherein the step (a) is conducted in the presence of an enhancing agent for the methylation; and

[7] The method of [6], wherein the enhancing agent for the methylation is S-adenosyl homocysteine hydrolase (SAHH).

In the present invention, methyltransferase activity of a WHSC1 or WHSC1L1 polypeptide can be determined by methods known in the art. For example, the WHSC1 or WHSC1L1 polypeptide and a substrate can be incubated with a labeled methyl donor, under a suitable assay condition. For example, a histone H3 peptide, and S-adenosyl-[methyl-¹⁴C]-L-methionine, or S-adenosyl-[methyl-³H]-L-methionine preferably can be used as such substrate and methyl donor, respectively. Transfer of the radiolabel to a histone H3 peptide can be detected, for example, by SDS-PAGE electrophoresis and fluorography. Alternatively, following the reaction, the histone H3 peptides can be separated from the methyl donor by filtration, and the amount of radiolabel retained on the filter quantitated by scintillation counting. Other suitable labels that can be attached to methyl donors, such as chromogenic and fluorescent labels, and methods of detecting transfer of these labels to histones and histone peptides, are known in the art.

Alternatively, the methyltransferase activity of WHSC1 or WHSC1L1 polypeptide can be determined using an unlabeled methyl donor (e.g., S-adenosyl-L-methionine) and reagents that selectively recognize methylated histones or histone peptides. For example, after incubation of the WHSC1 or WHSC1L1 polypeptide, a substrate and a methyl donor, under the condition capable of methylation of the substrate, the methylated substrate can be detected by immunological method. Any immunological techniques using an antibody that recognizes a methylated substrate can be used for the detection. For example, an antibody against a methylated histone is commercially available (abcam Ltd.). ELISA or Immunoblotting with an antibody that recognizes a methylated histone can be used for the present invention.

In the present invention, an enhancing agent for the methylation of a substance can be used. SAHH or functional equivalent thereof are one of the preferable enhancing agents for the methylation. The agent enhances the methylation of the substance, the methyltransferase activity can be determined with higher sensitivity thereby. WHSC1 or WHSC1L1 can be contacted with substrate and cofactor under the existence of the enhancing agent.

Furthermore, the present method detecting methyltransferase activity can be performed by preparing cells which express the WHSC1 or WHSC1L1 polypeptide, culturing the cells in the presence of a test substance, and determining methylation level of a histone, for example, by using the antibody specific binding to a methylation region.

More specifically, the method can include the steps of:

[1] contacting a test substance with cells expressing WHSC1 or WHSC1L1;

[2] detecting a methylation level of histone H3; and

[3] selecting the test substance that reduces the methylation level in the comparison with the methylation level detected in the absence of the test substance.

Alternatively, in some embodiments, the present invention also provides a method for evaluating or estimating a therapeutic effect of a test substance on treating or preventing cancer associated with over-expression of WHSC1 or WHSC1L1, the method including steps of:

(a) contacting a test substance with cells expressing WHSC1 or WHSC1L1 under the condition capable of methylation of histone H3

(b) detecting the methylation level of the histone H3; and

(c) correlating the potential therapeutic effect and the test substance, wherein the potential therapeutic effect is shown, when a test substance decreases the methylation level of the histone H3 as compared to the methylation level detected in the absence of the test substance as the candidate substance.

“Suppress the biological activity” as defined herein are preferably at least 10% suppression of the biological activity of WHSC1 or WHSC1L1 polypeptide in comparison with that in the absence of the substance, more preferably at least 25%, 50% or 75% suppression and further more preferably at 90% suppression.

In the preferred embodiments, control cells which do not express either or both of WHSC1 and WHSC1L1 polypeptide are used. Accordingly, the present invention also provides a method of screening for a candidate substance for inhibiting the growth of cells over-expressing either or both of WHSC1 and WHSCL1 or a candidate substance for treating or preventing a disease associated with either or both of WHSC1 and WHSC1L1 using either or both of the WHSC1 and WHSC1L1 polypeptide or a fragment thereof including the steps as follows:

a) culturing cells which express either or both of WHSC1 and WHSC1L1 polypeptide or a functional fragment thereof, and control cells that do not express either or both of WHSC1 and WHSC1L1 polypeptide or a functional fragment thereof in the presence of the test substance;

b) detecting the biological activity of the cells which express the protein and control cells; and

c) selecting the test compound that inhibits the biological activity in the cells which express the protein as compared to the proliferation detected in the control cells and in the absence of said test substance.

(iii) Screening for a Substance that Inhibits the Binding Between the WHSC1 Polypeptide and its Binding Proteins:

According to the present invention, it has been confirmed that the WHSC1 polypeptide interacts with the IQGAP1 polypeptide, TIAM1 polypeptide, AKT2 polypeptide and beta-catenin polypeptide. As those polypeptides are also known to be involved in carcinogenesis besides the involvement of WHSC1 polypeptide in carcinogenesis, the interaction between WHSC1 polypeptide and those polypeptides are considered to be important for cancer cell growth. Therefore, substances that inhibit the above interactions are expected to be useful for inhibiting cancer cell growth and/or survival, thus useful for treating or preventing cancer. Thus, the present invention provides methods of screening for candidate substances for treating or preventing cancer based on the binding activity of the WHSC1 polypeptide with the IQGAP1 polypeptide, TIAM1 polypeptide, AKT2 polypeptide or beta-catenin polypeptide. The present invention also provides methods of screening for a candidate substance for inhibiting cancer cell growth and/or survival. Specifically, the present screening methods include the steps as follows:

(a) contacting a IQGAP1 polypeptide, TIAM1 polypeptide, AKT2 polypeptide and/or beta-catenin polypeptide or functional equivalent thereof with a WHSC1 polypeptide or functional equivalent thereof in the presence of a test substance;

(b) detecting the binding between the IQGAP1 polypeptide, TIAM1 polypeptide, AKT2 polypeptide and/or beta-catenin polypeptide or functional equivalent thereof, and the WHSC1 polypeptide or functional equivalent thereof of the step (a); and

(c) selecting the test substance that inhibits the binding between the IQGAP1 polypeptide, TIAM1 polypeptide, AKT2 polypeptide and/or beta-catenin polypeptide or functional equivalent thereof, and the WHSC1 polypeptide or functional equivalent thereof as compared to the binding detected in the absence of the test substance.

Alternatively, the present screening methods include the steps as follows:

(a) contacting at least one of polypeptides selected from the group consisting of a IQGAP1 polypeptide, TIAM1 polypeptide, AKT2 polypeptide and beta-catenin polypeptide or functional equivalent thereof with a WHSC1 polypeptide or functional equivalent thereof in the presence of a test substance;
(b) detecting the binding between at least one of polypeptides selected from the group consisting of the IQGAP1 polypeptide, TIAM1 polypeptide, AKT2 polypeptide and beta-catenin polypeptide or functional equivalent thereof, and the WHSC1 polypeptide or functional equivalent thereof of the step (a); and
(c) selecting the test substance that inhibits the binding between at least one of polypeptides selected from the group consisting of the IQGAP1 polypeptide, TIAM1 polypeptide, AKT2 polypeptide and beta-catenin polypeptide or functional equivalent thereof, and the WHSC1 polypeptide or functional equivalent thereof as compared to the binding detected in the absence of the test substance.

According to the present invention, the therapeutic effect of the test substance on inhibiting the cancer cell growth or a candidate substance for treating or preventing WHSC1 associating disease can be evaluated. Therefore, the present invention also provides a method of screening for a candidate substance for inhibiting the cell growth or a candidate substance for treating or preventing WHSC1 associating disease, or a method of evaluating the therapeutic effect of the test substance on WHSC1 associating disease, using the WHSC1 polypeptide or functional equivalent thereof including the following steps:

(a) contacting a IQGAP1 polypeptide, TIAM1 polypeptide, AKT2 polypeptide and/or beta-catenin polypeptide or functional equivalent thereof with a WHSC1 polypeptide or functional equivalent thereof in the presence of a test substance;

(b) detecting the binding between the IQGAP1 polypeptide, TIAM1 polypeptide, AKT2 polypeptide and/or beta-catenin polypeptide or functional equivalent thereof with the WHSC1 polypeptide or functional equivalent thereof of the step (a); and

(c) correlating the binding of step (b) with the therapeutic effect of the test substance.

Alternatively, the present invention also provides a method of screening for a candidate substance for inhibiting the growth of cells over-expressing WHSC1 or a candidate substance for treating or preventing WHSC1 associating disease, or a method of evaluating the therapeutic effect of the test substance on WHSC1 associating disease, using the WHSC1 polypeptide or functional equivalent thereof including the following steps:

(a) contacting at least one of polypeptides selected from the group consisting of a IQGAP1 polypeptide, TIAM1 polypeptide, AKT2 polypeptide and beta-catenin polypeptide or functional equivalent thereof with a WHSC1 polypeptide or functional equivalent thereof in the presence of a test substance;

(b) detecting the binding between at least one of polypeptides selected from the group consisting of the IQGAP1 polypeptide, TIAM1 polypeptide, AKT2 polypeptide and beta-catenin polypeptide or functional equivalent thereof with the WHSC1 polypeptide or functional equivalent thereof of the step (a); and

(c) correlating the binding of step (b) with the therapeutic effect of the test substance.

Alternatively, in some embodiments, the present invention also provides a method for evaluating or estimating a therapeutic effect of a test substance on treating or preventing cancer, the method including steps of:

(a) contacting at least one of polypeptides selected from the group consisting of IQGAP1 polypeptide, TIAM1 polypeptide, AKT2 polypeptide and beta-catenin polypeptide or functional equivalent thereof with a WHSC1 polypeptide or functional equivalent thereof in the presence of a test substance;

(b) detecting the binding between at least one of polypeptides selected from the group consisting of the IQGAP1 polypeptide, TIAM1 polypeptide, AKT2 polypeptide and beta-catenin polypeptide or functional equivalent thereof with the WHSC1 polypeptide or functional equivalent thereof of the step (a); and

(c) comparing the binding level detected in the step (b) with those detected in the absence of the test substance; and

(d) correlating the potential therapeutic effect and the test substance, wherein the potential therapeutic effect is shown, when a test substance reduce the binding level.

In the present invention, the therapeutic effect can be correlated with the binding activity of the WHSC1 polypeptide or functional equivalent thereof to at least one of polypeptides selected from the group consisting of the IQGAP1 polypeptide, TIAM1 polypeptide, AKT2 polypeptide and beta-catenin polypeptide or a functional thereof. For example, when the test substance suppresses or inhibits the binding activity of the WHSC1 polypeptide to the above polypeptides as compared to the binding activity detected in the absence of the test substance, the test substance can identified or selected as the candidate substance having the therapeutic effect. Alternatively, when the test substance does not suppress or inhibit binding activity of the WHSC1 polypeptide to the above polypeptides as compared to a level detected in the absence of the test substance, the test substance is identified as having no significant therapeutic effect.

In the present results indicate that suppressing the binding activity among the WHSC1 polypeptide with the IQGAP1 polypeptide, TIAM1 polypeptide, AKT2 polypeptide and/or beta-catenin polypeptide or a functional equivalent thereof can reduce cancer cell growth. Thus, by screening for candidate substances that suppress binding activity, candidate substances that have the potential to treat or prevent cancers can be identified. The potential of these candidate substances to treat or prevent cancers can be evaluated by second and/or further screening to identify therapeutic substance for cancers.

According to the present invention, it was found that WHSC1 polypeptide interacted with IQGAP1 polypeptide, TIAM1 polypeptide, AKT2 polypeptide and/or beta-catenin polypeptide. Therefore, the present invention also provides a method of screening for a substance that inhibit the binding between WHSC1 polypeptide, and IQGAP1 polypeptide, TIAM1 polypeptide, AKT2 polypeptide and/or beta-catenin polypeptide, including the steps of:

(a) contacting a IQGAP1 polypeptide, TIAM1 polypeptide, AKT2 polypeptide and/or beta-catenin polypeptide or functional equivalent thereof with a WHSC1 polypeptide or functional equivalent thereof in the presence of a test substance;

(b) detecting the binding between the IQGAP1 polypeptide, TIAM1 polypeptide, AKT2 polypeptide and/or beta-catenin polypeptide or functional equivalent thereof, and the WHSC1 polypeptide or functional equivalent thereof; and

(c) selecting the test substance that inhibits the binding between the IQGAP1 polypeptide, TIAM1 polypeptide, AKT2 polypeptide and/or beta-catenin polypeptide or functional equivalent thereof, and the WHSC1 polypeptide or functional equivalent thereof as compared to the binding detected in the absence of the test substance.

Alternatively, the present invention also provides a method of screening for a substance that inhibit the binding between WHSC1 polypeptide, and at least one of polypeptides selected from the group consisting of IQGAP1 polypeptide, TIAM1 polypeptide, AKT2 polypeptide and beta-catenin polypeptide, including the steps of:

(a) contacting at least one of polypeptides selected from the group consisting of a IQGAP1 polypeptide, TIAM1 polypeptide, AKT2 polypeptide and beta-catenin polypeptide or functional equivalent thereof with a WHSC1 polypeptide or functional equivalent thereof in the presence of a test substance;

(b) detecting the binding between at least one of polypeptides selected from the group consisting of the IQGAP1 polypeptide, TIAM1 polypeptide, AKT2 polypeptide and beta-catenin polypeptide or functional equivalent thereof, and the WHSC1 polypeptide or functional equivalent thereof; and

(c) selecting the test substance that inhibits the binding between at least one of polypeptides selected from the group consisting of the IQGAP1 polypeptide, TIAM1 polypeptide, AKT2 polypeptide and beta-catenin polypeptide or functional equivalent thereof, and the WHSC1 polypeptide or functional equivalent thereof as compared to the binding detected in the absence of the test substance.

IQGAP1 polypeptide, TIAM1 polypeptide, AKT2 polypeptide and beta-catenin polypeptide to be used in the screening method of the present invention can be prepared based on the amino acid sequence data of the polypeptides or the nucleotide sequence data of the genes encoding those polypeptide by the methods well-known in the art as described below.

IQGAP1 (IQ motif containing GTPase activating protein 1provided) is a member of the IQGAP family. The IQGAP1 polypeptide is a 190-kDa protein and contains four IQ domains, one calponin homology domain, one Ras-GAP domain and one WW domain. It interacts with components of the cytoskeleton, with cell adhesion molecules, and with several signaling molecules to regulate cell morphology and motility. IQGAP1 polypeptide is likely related to cancer cell invasion. The typical nucleotide sequence of IQGAP1 gene and the typical amino acid sequence of IQGAP1 polypeptide are shown in SQ ID NO: 39 and SEQ ID NO: 40, respectively. These sequence data are also available from Genbank™ Accession No. NM_{—}003870.

TIAM1 (T-cell lymphoma invasion and metastasis 1) has been identified as an invasion- and metastasis-inducing gene in a murine T-lymphoma cell line. TIAM1 is Rac-specific guanine nucleotide exchange factor and specifically activates the Rho-like GTPase Rac. TIAM1-Rac signaling affects cell migration, invasion, and metastasis of cancer cells. The typical nucleotide sequence of TIAM1 gene and the typical amino acid sequence of IQGAP1 polypeptide are shown in SQ ID NO: 41 and SEQ ID NO: 42, respectively. These sequence data are also available from Genbank™ Accession No. NM_{—}003253.

AKT2 (v-akt murine thymoma viral oncogene homolog 2) is a putative oncogene encoding a protein belonging to a subfamily of serine/threonine kinases containing SH2-like (Src homology 2-like) domains. The AKT2 polypeptide is a general protein kinase capable of phosphorylating several known proteins. The typical nucleotide sequence of AKT2 gene and the typical amino acid sequence of AKT2 polypeptide are shown in SEQ ID NO: 43 and SEQ ID NO: 44, respectively. These sequence data are also available from Genbank™ Accession No. NM_{—}001626.

Beta-catenin is a part of a complex of proteins that constitute adherens junctions (AJs). Beta-catenin is involved in the Wnt/beta-catenin signaling pathway and the abnormal activation of Wnt/beta-catenin signaling pathway is considered to induce carcinogenesis. The typical nucleotide sequence of beta-catenin gene and the typical amino acid sequence of beta-catenin polypeptide are shown in SEQ ID NO: 45, 51 or 52 and SEQ ID NO: 46, respectively. These sequence data are also available from Genbank™ Accession No. NM_{—}001098209, NM_{—}001098210 or NM_{—}001904.

As a method of screening for substances that inhibit the binding between the WHSC1 polypeptide, and the IQGAP1 polypeptide, TIAM1 polypeptide, AKT2 polypeptide and/or beta-catenin polypeptide, many methods well known by one skilled in the art can be used. Alternatively, many methods well known by one skilled in the art can be used for screening for substances that inhibit the binding between the WHSC1 polypeptide, and at least one of polypeptides selected from the group consisting of IQGAP1, TIAM1, AKT2, and beta-catenin. For example, screening can be carried out as an in vitro assay system, such as a cellular system. More specifically, first, either the WHSC1 polypeptide, or the IQGAP1 polypeptide, TIAM1 polypeptide, AKT2 polypeptide and/or beta-catenin polypeptide is bound to a support, and the other protein is added together with a test substance thereto. Next, the mixture is incubated, washed and the other protein bound to the support is detected and/or measured.

Examples of supports that can be used for binding proteins include, for example, insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials can be used. When using beads, they can be filled into a column. Alternatively, the use of magnetic beads is also known in the art, and enables one to readily isolate proteins bound on the beads via magnetism.

The binding of a protein to a support can be conducted according to routine methods, such as chemical bonding and physical adsorption, for example. Alternatively, a protein can be bound to a support via antibodies that specifically recognize the protein. Moreover, binding of a protein to a support can be also conducted by means of avidin and biotin.

The binding between proteins is preferably carried out in buffer, examples of which include, but are not limited to, phosphate buffer and Tris buffer. However, the selected buffer must not inhibit binding between the proteins.

In the context of the present invention, a biosensor using the surface plasmon resonance phenomenon can be used as a mean for detecting or quantifying the bound protein. When such a biosensor is used, the interaction between the proteins can be observed in real-time as a surface plasmon resonance signal, using only a minute amount of polypeptide and without labeling (for example, BIAcore, Pharmacia). Therefore, it is possible to evaluate binding between the WHSC1 polypeptide, and IQGAP1 polypeptide, TIAM1 polypeptide, AKT2 polypeptide and/or beta-catenin polypeptide using a biosensor such as BIAcore.

Alternatively, either the WHSC1 polypeptide, or the IQGAP1 polypeptide, TIAM1 polypeptide, AKT2 polypeptide and/or beta-catenin polypeptide can be labeled, and the label of the bound protein can be used to detect or measure the bound protein. Specifically, after pre-labeling one of the proteins, the labeled protein is contacted with the other protein in the presence of a test substance, and then bound proteins are detected or measured according to the label after washing.

Labeling substances including but not limited to radioisotope (e.g., ³H, ¹⁴C, ³²P, ³³P, ³⁵S, ¹²⁵I, ¹³¹I), enzymes (e.g., alkaline phosphatase, horseradish peroxidase, beta-galactosidase, beta-glucosidase), fluorescent substances (e.g., fluorescein isothiocyanate (FITC), rhodamine) and biotin/avidin can be used for the labeling of a protein in the present method. When the protein is labeled with a radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein can be detected or measured using fluorophotometer.

Furthermore, the binding of the WHSC1 polypeptide, and IQGAP1 polypeptide, TIAM1 polypeptide, AKT2 polypeptide and/or beta-catenin polypeptide can be also detected or measured using antibodies to the polypeptide thereof. For example, after contacting the WHSC1 polypeptide immobilized on a support with a test substance and the IQGAP1 polypeptide, TIAM1 polypeptide, AKT2 polypeptide and/or beta-catenin polypeptide, the mixture is incubated and washed, and detection or measurement can be conducted using an antibody against the IQGAP1 polypeptide, TIAM1 polypeptide, AKT2 polypeptide or beta-catenin polypeptide. Alternatively, the IQGAP1 polypeptide, TIAM1 polypeptide, AKT2 polypeptide and/or beta-catenin polypeptide can be immobilized on a support, and an antibody against the WHSC1 polypeptide can be used as the antibody.

When using an antibody in the present screening, the antibody is preferably labeled with one of the labeling substances mentioned above, and detected or measured based on the labeling substance. Alternatively, an antibody against the WHSC1 polypeptide, IQGAP1 polypeptide, TIAM1 polypeptide, AKT2 polypeptide or beta-catenin polypeptide can be used as a primary antibody to be detected with a secondary antibody that is labeled with a labeling substance. Furthermore, an antibody bound to the protein in the screening of the present invention can be detected or measured using a protein G or protein A column.

The polypeptides to be used in the present screening methods can be recombinantly produced using standard procedures. For example, a gene encoding a polypeptide of interest can be expressed in animal cells by inserting the gene into an expression vector for foreign genes, such as pSV2neo, pcDNA I, pcDNA3.1, pCAGGS and pCD8. The promoter to be used for the expression can be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466-72 (1988)), the CMV immediate early promoter (Seed and Aruffo, Proc Natl Acad Sci USA 84: 3365-9 (1987)), the SV40 late promoter (Gheysen and Fiers, J Mol Appl Genet. 1: 385-94 (1982)), the Adenovirus late promoter (Kaufman et al., Mol Cell Biol 9: 946-58 (1989)), the HSV TK promoter and so on. The introduction of the gene into animal cells to express a foreign gene can be performed according to any conventional method, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B, Cell 76: 1025-37 (1994); Lamb et al., Nature Genetics 5: 22-30 (1993): Rabindran et al., Science 259: 230-4 (1993)), and so on. A polypeptide can be expressed as a fusion protein including a recognition site (epitope) of a monoclonal antibody by introducing the epitope of the monoclonal antibody, whose specificity has been revealed, to the N- or C-terminus of the polypeptide. Alternatively, a commercially available epitope-antibody system can be used (Experimental Medicine 13: 85-90 (1995)). Vectors which are capable of expressing a fusion protein with, for example, beta-galactosidase, maltose binding protein, glutathione S-transferase, green florescence protein (GFP), and so on, by the use of its multiple cloning sites are commercially available.

A fusion protein, prepared by introducing only small epitopes composed of several to a dozen amino acids so as not to change the property of the original polypeptide by the fusion, is also provided herein. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and antibodies recognizing them can be used as the epitope-antibody system for detecting the binding activity between the polypeptides (Experimental Medicine 13: 85-90 (1995)).

Antibodies to be used in the present screening methods can be prepared using techniques well known in the art. Antigens to prepared antibodies can be derived from any animal species, but preferably is derived from a mammal such as a human, mouse, rabbit, or rat, more preferably from a human. The polypeptide used as the antigen can be recombinantly produced or isolated from natural sources. The polypeptides to be used as an immunization antigen can be a complete protein or a partial peptide derived from the complete protein.

Any mammalian animal can be immunized with the antigen; however, the compatibility with parental cells used for cell fusion is preferably taken into account. In general, animals of the order Rodentia, Lagomorpha or Primate are used. Animals of the Rodentia order include, for example, mice, rats and hamsters. Animals of Lagomorpha order include, for example, hares, pikas, and rabbits. Animals of Primate order include, for example, monkeys of Catarrhini (old world monkey) such as Macaca fascicularis, rhesus monkeys, sacred baboons and chimpanzees.

Methods for immunizing animals with antigens are well known in the art. Intraperitoneal injection or subcutaneous injection of antigens is a standard method for immunizing mammals. More specifically, antigens can be diluted and suspended in an appropriate amount of phosphate buffered saline (PBS), physiological saline, etc. If desired, the antigen suspension can be mixed with an appropriate amount of a standard adjuvant, such as Freund's complete adjuvant, made into emulsion, and then administered to mammalian animals. Preferably, it is followed by several administrations of the antigen mixed with an appropriate amount of Freund's incomplete adjuvant every 4 to 21 days. An appropriate carrier can also be used for immunization. After immunization as above, the serum is examined by a standard method for an increase in the amount of desired antibodies.

Polyclonal antibodies can be prepared by collecting blood from the immunized mammal examined for the increase of desired antibodies in the serum, and by separating serum from the blood by any conventional method. Polyclonal antibodies include serum containing the polyclonal antibodies, as well as the fraction containing the polyclonal antibodies isolated from the serum. Immunoglobulin G or M can be prepared from a fraction which recognizes only the objective polypeptide using, for example, an affinity column coupled with the polypeptide, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion are preferably obtained from spleen. Other preferred parental cells to be fused with the above immune cell include, for example, myeloma cells of mammals, and more preferably myeloma cells having an acquired property for the selection of fused cells by drugs.

The above immune and myeloma cells can be fused according to known methods, for example, the method of Milstein et al., (Galfre and Milstein, Methods Enzymol 73: 3-46 (1981)).

Resulting hybridomas obtained by the cell fusion can be selected by cultivating them in a standard selection medium, such as HAT medium (hypoxanthine, aminopterin, and thymidine containing medium). The cell culture is typically continued in the HAT medium for several days to several weeks, the time being sufficient to allow all the other cells, with the exception of the desired hybridoma (non-fused cells), to die. Then, the standard limiting dilution is performed to screen and clone a hybridoma cell producing the desired antibody.

In addition to the above method, in which a non-human animal is immunized with an antigen for preparing hybridoma, human lymphocytes, such as those infected by the EB virus, can be immunized with an antigen, cells expressing such antigen, or their lysates in vitro. Then, the immunized lymphocytes are fused with human-derived myeloma cells that are capable of indefinitely dividing, such as U266, to yield a hybridoma producing a desired human antibody that is able to bind to the antigen (Unexamined Published Japanese Patent Application No. (JP-A) Sho 63-17688).

The obtained hybridomas can be subsequently transplanted into the abdominal cavity of a mouse and the ascites can be extracted. The obtained monoclonal antibodies can be purified by, for example, ammonium sulfate precipitation, a protein A or protein G column, DEAE ion exchange chromatography, or an affinity column carrying an objective antigen.

Antibodies against the WHSC1 polypeptide, IQGAP1 polypeptide, TIAM1 polypeptide, AKT2 polypeptide or beta-catenin polypeptide can be used not only in the present screening method, but also for the detection of the polypeptides as cancer markers in biological samples as described in “A method for diagnosing cancer”. They can further serve as candidates for agonists and antagonists of the polypeptides of interest. In addition, such antibodies, serving as candidates for antagonists, can be applied to the antibody treatment for diseases related to the WHSC1 polypeptide, including bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor as described infra.

Monoclonal antibodies thus obtained can be also recombinantly prepared using genetic engineering techniques (see, for example, Borrebaeck and Larrick, Therapeutic Monoclonal Antibodies, published in the United Kingdom by MacMillan Publishers LTD (1990)). For example, a DNA encoding an antibody can be cloned from an immune cell, such as a hybridoma or an immunized lymphocyte producing the antibody, inserted into an appropriate vector, and introduced into host cells to prepare a recombinant antibody. Such recombinant antibody can also be used in the context of the present screening.

Furthermore, antibodies used in the screening and so on can be fragments of antibodies or modified antibodies, so long as they retain the original binding activity. For instance, the antibody fragment can be an Fab, F(ab′)₂, Fv, or single chain Fv (scFv), in which Fv fragments from H and L chains are ligated by an appropriate linker (Huston et al., Proc Natl Acad Sci USA 85: 5879-83 (1988)). More specifically, an antibody fragment can be generated by treating an antibody with an enzyme, such as papain or pepsin. Alternatively, a gene encoding an antibody fragment can be constructed, inserted into an expression vector, and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9: 132-7 (1991)).

An antibody can be modified by conjugation with a variety of molecules, such as polyethylene glycol (PEG). Modified antibodies can be obtained through chemically modification of an antibody. These modification methods are conventional in the field.

Antibodies obtained as above can be purified to homogeneity. For example, the separation and purification of the antibody can be performed according to separation and purification methods used for general proteins. For example, the antibody can be separated and isolated by appropriately selected and combined column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)); however, the present invention is not limited thereto. A protein A column and protein G column can be used as the affinity column. Exemplary protein A columns to be used include, for example, Hyper D, POROS, and Sepharose F. F. (Pharmacia).

Exemplary chromatography, with the exception of affinity, includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

Alternatively, a two-hybrid system utilizing cells can be used for detecting or measuring the binding activity among the polypeptides (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, for example, the WHSC1 polypeptide are fused to the SRF-binding region or GAL4-binding region and expressed in yeast cells. The IQGAP1 polypeptide, TIAM1 polypeptide, AKT2 polypeptide or beta-catenin polypeptide are fused to the VP16 or GAL4 transcriptional activation region and also expressed in the yeast cells in the existence of a test substance. Alternatively, the IQGAP1 polypeptide, TIAM1 polypeptide, AKT2 polypeptide polypeptide or beta-catenin polypeptide may be fused to the SRF-binding region or GAL4-binding region, and the WHSC1 polypeptide can be fused to the VP16 or GAL4 transcriptional activation region. The binding of the two polypeptides activates a reporter gene, making positive clones detectable. As a reporter gene, for example, Ade2 gene, lacZ gene, CAT gene, luciferase gene and such can be used besides HIS3 gene.

(2) Gene Expression Based Screening:

(i) Screening for a Substance Altering the Expression of WHSC1 or WHSC1L1 Gene:

In the present invention, the decrease of the expression of WHSC1 or WHSC1L1 by siRNA inhibits cancer cell proliferation (FIG. 4). Therefore, the present invention provides a method of screening for a substance that inhibits the expression of WHSC1 or WHSC1L1 gene. A substance that inhibits the expression of WHSC1 or WHSC1L1 gene is expected to suppress the proliferation of cancer cells, and thus is useful for treating or preventing cancer. For example, substances that inhibit the expression of WHSC1 can be candidate therapeutic agents for treatment or prevention of cancers, including, but not limited to, bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor. Also, substances that inhibit the expression of WHSC1L1 can be candidate therapeutic agents for treatment or prevention of cancers, including but not limited to, bladder cancer, breast cancer, CML, lung cancer (e.g., SCLC) and lymphoma. Therefore, the present invention also provides a method for screening a substance that suppresses the proliferation of cancer cells, and a method for screening a candidate substance for treating or preventing cancer. In the context of the present invention, such screening method can include, for example, the following steps:

(a) contacting a test substance with a cell expressing either or both of WHSC1 and WHSC1L1 gene;

(b) detecting either of the expression level of WHSC1 gene or the expression level of WHSC1L1 gene, or both; and

(b) selecting the test substance that reduces either of the expression level of WHSC1 gene or the expression level of WHSC1L1 gene, or both as compared to the expression level(s) detected in the absence of the test substance.

Alternatively, in some embodiments, the present invention also provides a method for evaluating or estimating a therapeutic effect of a test substance on treating or preventing cancer associated with over-expression of WHSC1 or WHSC1L1, the method including steps of:

(a) contacting a test substance with a cell expressing either or both of WHSC1 and WHSC1L1 gene; and;

(b) correlating the potential therapeutic effect and the test substance, wherein the potential therapeutic effect is shown, when a substance reduces either of the expression level of WHSC1 gene or the expression level of WHSC1L1 gene, or both as compared to a control.

The method of the present invention will be described in more detail below.

Cells expressing the WHSC1 gene include, for example, cell lines established from bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor. Also, cells expressing the WHSC1L1 gene include, for example, cell lines established from bladder cancer, breast cancer CML, lung cancer (e.g., SCLC) and lymphoma. Such cells can be used for the above screening of the present invention. The expression level of WHSC1 or WHSC1L1 gene can be estimated by methods well known to one skilled in the art, for example, RT-PCR, Northern blot assay, Western blot assay, immunostaining and flow cytometry analysis. “Reduce the expression level” as defined herein are preferably at least 10% reduction of the expression level of WHSC1 or WHSC1L1 gene in comparison to the expression level in the absence of the substance, more preferably at least 25%, 50% or 75% reduced level and further more preferably at least 95% reduced level. Substances herein include chemical compounds, double-strand molecules against WHSC1 or WHSC1L1 gene (e.g., siRNA), antisense nucleic acids against WHSC1 or WHSC1L1 gene and so on. The preparation methods of the double-strand nucleotides are described in the following section. In the screening method of the present invention, a substance that reduces the expression level of WHSC1 or WHSC1L1 gene can be selected as candidate substances to be used for the treatment or prevention of cancer.

Alternatively, the screening method of the present invention can include the following steps:

(a) contacting a test substance with a cell into which a vector, including the transcriptional regulatory region of WHSC1 or WHSC1L1 gene and a reporter gene that is expressed under the control of the transcriptional regulatory region, has been introduced;

(b) measuring the expression or activity level of the reporter gene; and

(c) selecting the test substance that reduces the expression or activity of said reporter gene as compared to the expression or activity detected in the absence of the test substance.

Alternatively, in some embodiments, the present invention also provides a method for evaluating or estimating a therapeutic effect of a test substance on treating or preventing cancer associated with over-expression of WHSC1 or WHSC1L1, the method includes the steps of:

(a) contacting a test substance with a cell into which a vector, including the transcriptional regulatory region of WHSC1 or WHSC1L1 gene and a reporter gene that is expressed under the control of the transcriptional regulatory region, has been introduced;

(b) measuring the expression or activity of said reporter gene; and

(c) correlating the potential therapeutic effect and the test substance, wherein the potential therapeutic effect is shown, when a test substance reduces the expression or activity of said reporter gene.

Suitable reporter genes and host cells are well known in the art. For example, reporter genes include luciferase, green florescence protein (GFP), Discosoma sp. Red Fluorescent Protein (DsRed), Chrolamphenicol Acetyltransferase (CAT), lacZ and beta-glucuronidase (GUS), and host cells include COS7, HEK293, HeLa and so on. The reporter construct required for the screening can be prepared by connecting reporter gene sequence to the transcriptional regulatory region of WHSC1 or WHSC1L1 gene. The transcriptional regulatory region of WHSC1 or WHSC1L1 gene herein is the region from transcription stat site to at least 500 bp upstream, preferably 1,000 bp, more preferably 5,000 or 10,000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the reporter construct is introduced into host cells and the expression or activity of the reporter gene is detected by methods well known in the art (e.g., using luminometer, absorption spectrometer, flow cytometer and so on). “Reduces the expression or activity” as defined herein are preferably at least 10% reduction of the expression or activity of the reporter gene in comparison with in absence of the substance, more preferably at least 25%, 50% or 75% reduction and further more preferably at least 95% reduction.

By screening for test substances that (i) bind to the WHSC1 or WHSC1L1 polypeptide; (ii) suppress/reduce the biological activity (e.g., cell-proliferating activity or methyltransferase activity) of the WHSC1 or WHSC1L1 polypeptide; or (iii) reduce the expression level of WHSC1 or WHSC1L1 gene, candidate substances that have the potential to treat or prevent cancers (e.g., bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma) can be identified. Potential of these candidate substances to treat or prevent cancers can be evaluated by second and/or further screening to identify therapeutic substance for cancers. For example, when a substance that binds to the WHSC1 or WHSC1L1 polypeptide inhibits the above-described activities of WHSC1 or WHSC1L1 polypeptide, it can be concluded that such a substance has the specific therapeutic effect for cancer associated with WHSC1 and/or WHSC1L1 overexpression.

In the present invention, the downstream genes regulated by WHSC1 were identified. WHSC1 polypeptide is involved in a pathway relating carcinogenesis, as the suppression of WHSC1 expression level by siRNA inhibited cancer cell proliferation. Accordingly, a substance that regulates the expression level of a downstream gene may be useful for treating or preventing cancer. Therefore, the present invention also provides a method of screening for a candidate substance for treating or preventing cancer, such as bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor. In one embodiment, the method of screening for a candidate substance for treating or preventing cancer can include the steps of:

(a) contacting a test substance with a cell expressing WHSC1 and a downstream gene of WHSC1; and

(b) selecting the substance that reduces expression level of a downstream gene of WHSC1 in comparison with the expression level detected in the absence of the candidate substance.

Alternatively, in some embodiments, the present invention also provides a method for evaluating or estimating a therapeutic effect of a test substance on treating or preventing cancer associated with over-expression of WHSC1, the method including steps of:

(a) contacting a test substance with a cell expressing WHSC1 and a downstream gene of WHSC1; and

(b) correlating the potential therapeutic effect and the test substance, wherein the potential therapeutic effect is shown, when a substance reduces expression level of a downstream gene of WHSC1 as compared to a control.

Furthermore, in the present invention, the genes indicated in Table 8 were identified as downstream genes regulated by WHSC1. Therefore, the downstream genes to be detected by expression level in the method of the present invention can be one or more genes described in Table 8. For example, the expression of CCND1 was confirmed to be regulated by the regulation of WHSC1 expression (FIG. 6).

Accordingly, the present invention provides the method of screening for a candidate substance for treating or preventing cancer, the method including the steps of:

(a) contacting a test substance with a cell expressing WHSC1 and CCND1; and

(b) selecting the test substance that reduces expression level of CCND1 in comparison with the expression level detected in the absence of the test substance.

Alternatively, in some embodiments, the present invention also provides a method for evaluating or estimating a therapeutic effect of a test substance on treating or preventing cancer associated with over-expression of WHSC1, the method including the steps of:

(a) contacting a test substance with a cell expressing WHSC1 and CCND1; and;

(b) correlating the potential therapeutic effect and the test substance, wherein the potential therapeutic effect is shown, when a substance reduces the expression level of CCND1 as compared to a control.

The expression level of a CCND1 gene can be detected by the methods well known in the art such as methods described above (see, “A method for diagnosing cancer”), using the nucleotide and/or amino acid sequence data of CCND1. A typical nucleotide sequence of CCND1 is shown in SEQ ID NO: 47, and a typical amino acid sequence is shown in SEQ ID NO: 48. These sequence data are also available from Genbank Accession No. NM_{—}053056.

Double Stranded Molecule:

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g. doublestranded chimera of DNA and RNA (dsD/R-NA) or small hairpin chimera of DNA and RNA (shD/R-NA)).

As used herein, a target sequence is a nucleotide sequence within mRNA or cDNA sequence of a target gene, which will result in suppression of translation of the whole mRNA of the target gene if the double-stranded molecule is introduced within a cell expressing the gene. A nucleotide sequence within mRNA or cDNA sequence of a target gene can be determined to be a target sequence when a double-stranded molecule having a sequence corresponding to the target sequence inhibits expression of the gene in a cell expressing the gene. When a target sequence is shown by cDNA sequence, a sense strand sequence of a double-stranded cDNA, i.e., a sequence that mRNA sequence is converted into DNA sequence, is used for defining a target sequence. A double-stranded molecule is composed of a sense strand that has a sequence corresponding to a target sequence and an antisense strand that has a complementary sequence to the target sequence, and the antisense strand hybridizes with the sense strand at the complementary sequence to form a double-stranded molecule.

Herein, the phrase “corresponding to” means converting a target sequence to the kind of nucleic acid that constitutes a sense strand of a double-stranded molecule. For example, when a target sequence is shown in DNA sequence and a sense strand of a double-stranded molecule has an RNA region, base “t”s within the RNA region is replaced with base “u”s. On the other hand, when a target sequence is shown in RNA sequence and a sense strand of a double-stranded molecule has a DNA region, base “u”s within the DNA region is replaced with “t”s. For example, when a target sequence is the DNA sequence shown in SEQ ID NO: 29, 32, 35 or 38 and the sense strand of the double-stranded molecule is composed of RNA, “a sequence corresponding to a target sequence” is “CAGAUCUACA CAGCGGAUA” (for SEQ ID NO: 29), “GUUAAUUGGC AUAUGGAAU” (for SEQ ID NO: 32), “CUCACAAAUG GGUAUCCAU” (for SEQ ID NO: 35), or “GUACUGAAAU UCGGAGACA” (for SEQ ID NO: 38).

Also, a complementary sequence to a target sequence for an antisense strand of a double-stranded molecule can be defined according to the kind of nucleic acid that constitutes the antisense strand. For example, when a target sequence is the DNA sequence shown in SEQ ID NO: 29, 32, 35 or 38 and the antisense strand of the double-stranded molecule is composed of RNA, “a complementary sequence to a target sequence” is “UAUCCGCUG UGUAGAUCUG” (for SEQ ID NO: 29), “AUUCCAUAU GCCAAUUAAC” (for SEQ ID NO: 32), “AUGGAUACC CAUUUGUGAG” (for SEQ ID NO: 35) or “UGUCUCCGA AUUUCAGUAC” (for SEQ ID NO: 38). A double-stranded molecule can have one or two 3′ overhangs having 2 to 5 nucleotides in length (e.g., uu) and/or a loop sequence that links a sense strand and an antisense strand to form hairpin structure, in addition to a sequence corresponding to a target sequence and complementary sequence thereto.

As used herein, the term “siRNA” refers to a double-stranded RNA molecule which prevents translation of a target mRNA. Standard techniques of introducing siRNA into the cell are used, including those in which DNA is a template from which RNA is transcribed. Alternatively, siRNA can also be directly introduced in cells to be treated. Methods of introducing siRNA in a subject are well known in the art. For example, an administration of siRNA in conjunction with a delivery substance is preferable for the introductionod siRNA.

The siRNA includes a WHSC1 or WHSC1L1 sense nucleic acid sequence (also referred to as “sense strand”), a WHSC1 or WHSC1L1 antisense nucleic acid sequence (also referred to as “antisense strand”) or both. The siRNA can be constructed such that a single transcript has both the sense and complementary antisense nucleic acid sequences of the target gene, e.g., a hairpin. The siRNA can either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands can include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions are sufficient such that base pairing occurs between the regions, the first and second regions are joined by a loop region, the loop results from a lack of base pairing between nucleotides (or nucleotide analogs) within the loop region. The loop region of an shRNA is a single-stranded region intervening between the sense and antisense strands and can also be referred to as “intervening single-strand”.

As used herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule can contain RNA and DNA. Standard techniques of introducing siD/R-NA into the cell are used. The siD/R-NA includes a WHSC1 or WHSC1L1 sense nucleic acid sequence (also referred to as “sense strand”), a WHSC1 or WHSC1L1 antisense nucleic acid sequence (also referred to as “antisense strand”) or both. The siD/R-NA can be constructed such that a single transcript has both the sense and complementary antisense nucleic acid sequences from the target gene, e.g., a hairpin. The siD/R-NA can either be a dsD/R-NA or shD/R-NA.

As used herein, sense strand of a target sequence is a nucleotide sequence within mRNA or cDNA sequence of a gene, which will result in suppression of translation of the whole mRNA if a double-stranded nucleic acid molecule of the invention was introduced within a cell expressing the gene. A nucleotide sequence within mRNA or cDNA sequence of a gene can be determined to be a target sequence when a doublestranded polynucleotide comprising a sequence corresponding to the target sequence inhibits expression of the gene in a cell expressing the gene. The double stranded polynucleotide which suppresses the gene expression can consist of the target sequence and 3′ overhang having 2 to 5 nucleotides in length (e.g., uu).

As used herein, the term “dsD/R-NA” refers to a construct of two molecules composed of complementary sequences to one another and that have annealed together via the complementary sequences to form a double-stranded polynucleotide molecule. The nucleotide sequence of two strands can include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of a first and second regions complementary to one another, i.e., sense and antisense strands. The degree of complementarity and orientation of the regions are sufficient such that base pairing occurs between the regions, the first and second regions are joined by a loop region, the loop results from a lack of base pairing between nucleotides (or nucleotide analogs) within the loop region. The loop region of an shD/R-NA is a single-stranded region intervening between the sense and antisense strands and can also be referred to as “intervening single-strand”.

As used herein, an “isolated nucleic acid” is a nucleic acid removed from its original environment (e.g., the natural environment if naturally occurring) and thus, synthetically altered from its natural state. In the present invention, examples of isolated nucleic acid includes DNA, RNA, and derivatives thereof.

A double-stranded molecule directed against WHSC1 or WHSC1L1, which molecule hybridizes to target mRNA, decreases or inhibits production of WHSC1 or WHSC1L1 protein encoded by WHSC1 or WHSC1L1 gene by associating with the normally single-stranded mRNA transcript of the gene, thereby interfering with translation and thus, inhibiting expression of the protein. As demonstrated herein, the expression of WHSC1 or WHSC1L1 in several cancer cell lines was inhibited by dsRNA (FIG. 4). Therefore, the present invention provides isolated double-stranded molecules that are capable of inhibiting the expression of the WHSC1 or WHSC1L1 gene when introduced into a cell expressing the gene. The target sequence of double-stranded molecule can be designed by an siRNA design algorithm such as that mentioned below.

Target sequences for WHSC1 gene include, for example, nucleotide sequences of SEQ ID NO: 29 and 32, and target sequences include, for example, nucleotide sequence of SEQ ID NO: 35 and 38.

Specifically, the present invention provides the following double-stranded molecules of [1] to [18]:

[1] An isolated double-stranded molecule that, when introduced into a cell, inhibits in vivo expression of WHSC1 or WHSC1L1 and cell proliferation, such molecules composed of a sense strand and an antisense strand complementary thereto, hybridized to each other to form the double-stranded molecule;

[2] The double-stranded molecule of [1], wherein said double-stranded molecule acts on mRNA, matching a target sequence of SEQ ID NO: 29, 32, 35 or 38

[3] The double-stranded molecule of [1], wherein the sense strand contains a nucleotide sequence corresponding to a target sequence of SEQ ID NO: 29, 32, 35 or 38;

[4] The double-stranded molecule of [3], having a length of less than about 100 nucleotides;

[5] The double-stranded molecule of [4], having a length of less than about 75 nucleotides;

[6] The double-stranded molecule of [5], having a length of less than about 50 nucleotides;

[7] The double-stranded molecule of [6] having a length of less than about 25 nucleotides;

[8] The double-stranded molecule of [7], having a length of between about 19 and about 25 nucleotides;

[9] The double-stranded molecule of any one of [1] to [8], composed of a single polynucleotide having both the sense and antisense strands linked by an intervening single-strand;

[10] The double-stranded molecule of [9], having the general formula of 5′-[A]-[B]-[A′]-3′ or 5′-[A′]-[B]-[A]-3, wherein [A] is the sense strand containing a sequence corresponding to a target sequence of SEQ ID NO: 29, 32, 35 or 38, [B] is the intervening single-strand composed of 3 to 23 nucleotides, and [A′] is the antisense strand containing a sequence complementary to [A];

[11] The double-stranded molecule of any one of [1] to [10], composed of RNA;

[12] The double-stranded molecule of any one of [1] to [10], composed of both DNA and RNA;

[13] The double-stranded molecule of [12], wherein the molecule is a hybrid of a DNA polynucleotide and an RNA polynucleotide;

[14] The double-stranded molecule of [13] wherein the sense and the antisense strands are composed of DNA and RNA, respectively;

[15] The double-stranded molecule of [12], wherein the molecule is a chimera of DNA and RNA;

[16] The double-stranded molecule of [15], wherein a region flanking to the 3′-end of the antisense strand, or both of a region flanking to the 5′-end of sense strand and a region flanking to the 3′-end of antisense strand are RNA;

[17] The double-stranded molecule of [16], wherein the flanking region is composed of 9 to 13 nucleotides; and

[18] The double-stranded molecule of [2], wherein the molecule contains one or two 3′ overhangs.

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (www.ambion.com/techlib/misc/siRNA_finder.html).

The computer program selects target nucleotide sequences for double-stranded molecules based on the following protocol.

Selection of Target Sites:

1. Beginning with the AUG start codon of the transcript, scan downstream for AA dinucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Using the above protocol, the target sequence of the isolated double-stranded molecules of the present invention were designed as:

SEQ ID NO: 29 or 32 for WHSC1 gene; and

SEQ ID NO: 35 or 38 for WHSC1L1 gene.

Double-stranded molecules targeting the above-mentioned target sequences were respectively examined for their ability to suppress the growth of cells expressing the target genes. Therefore, the present invention provides double-stranded molecules targeting the sequences of SEQ ID NO: 29 or 32 for WHSC1 gene, or the sequences of SEQ ID NO: 35 or 38 for WHSC1L1 gene.

The double-stranded molecule of the present invention can be directed to a single target WHSC1 or WHSC1L1 gene sequence or can be directed to a plurality of target WHSC1 or WHSC1L1 gene sequences.

A double-stranded molecule of the present invention targeting the above-mentioned target sequence of WHSC1 or WHSC1L1 gene include isolated polynucleotide that contain the nucleic acid sequence corresponding to the target sequence and/or the complementary sequence to the target sequence. Examples of a polynucleotide targeting a WHSC1 gene includes one containing the sequence corresponding to the target sequence of SEQ ID NO: 29 or 32 and/or complementary sequence to such target sequence. Also, examples of a polynucleotide targeting a WHSC1L1gene includes one containing the sequence corresponding to the target sequence of SEQ ID NO: 35 or 38 and/or complementary sequence to such target sequence.

In an embodiment, a double-stranded molecule is composed of two polynucleotides, one polynucleotide has a sequence corresponding to a target sequence, i.e., sense strand, and another polypeptide has a complementary sequence to the target sequence, i.e., antisense strand. The sense strand polynucleotide and the antisense strand polynucleotide hybridize to each other to form double-stranded molecule. Examples of such double-stranded molecules include dsRNA and dsD/R-NA.

In an another embodiment, a double-stranded molecule is composed of a polynucleotide that has both a sequence corresponding to a target sequence, i.e., sense strand, and a complementary sequence to the target sequence, i.e., antisense strand. Generally, the sense strand and the antisense strand are linked by a intervening strand, and hybridize to each other to form a hairpin loop structure. Examples of such doublestranded molecule include shRNA and shD/R-NA.

In other words, a double-stranded molecule of the present invention is composed of a sense strand polynucleotide having a nucleotide sequence of the target sequence and anti-sense strand polynucleotide having a nucleotide sequence complementary to the target sequence, and both of polynucleotides hybridize to each other to form the double-stranded molecule. In the double-stranded molecule including the polynucleotides, a part of the polynucleotide of either or both of the strands can be RNA, and when the target sequence is defined with a DNA sequence, the nucleotide “t” within the target sequence and complementary sequence thereto is replaced with “u”.

In one embodiment of the present invention, such a double-stranded molecule of the present invention includes a stem-loop structure, composed of the sense and antisense strands. The sense and antisense strands can be joined by a loop. Accordingly, the present invention also provides the double-stranded molecule composed of a single polynucleotide containing both the sense strand and the antisense strand linked or flanked by an intervening single-strand.

In the present invention, double-stranded molecules targeting the WHSC1 or WHSC1L1 gene can have a sequence selected from among SEQ ID NOs: 29, 32, 35 and 38 as a target sequence. In preferred embodiments, the target sequence is a sequence of SEQ ID NO: 29, 32, 35 or 38. Accordingly, preferable examples of the double-stranded molecule of the present invention include polynucleotides that hybridize to each other at a sequence corresponding to SEQ ID NO: 29, 32, 35 or 38 and a complementary sequence thereto, and a polynucleotide that has a sequence corresponding to SEQ ID NO: 29, 32, 35 or 38 and a complementary sequence thereto.

However, the present invention is not limited to this example, and minor modifications in the aforementioned target sequences are acceptable so long as the modified molecule retains the ability to suppress the expression of WHSC1 or WHSC1L1 gene. Herein, the phrase “minor modification” as used in connection with a nucleic acid sequence indicates one, two or several substitution, deletion, addition or insertion to the sequence.

In the context of the present invention, the term “several” as applies to substitutions, deletions, additions and/or insertions in a nucleic acid sequence can mean 3-7, preferably 3-5, more preferably 3-4, even more preferably 3 nucleic acid residues.

According to the present invention, a double-stranded molecule of the present invention can be tested for its ability using the methods utilized in the Examples. In the Examples herein below, double-stranded molecules composed of sense strands of various portions of mRNA of WHSC1 or WHSC1L1 genes or antisense strands complementary thereto were tested in vitro for their ability to decrease production of WHSC1 or WHSC1L1 gene product in cancer cell lines according to standard methods. Furthermore, for example, reduction in WHSC1 or WHSC1L1 gene product in cells contacted with the candidate double-stranded molecule compared to cells cultured in the absence of the candidate molecule can be detected by, e.g., RT-PCR using primers for WHSC1 or WHSC1L1 mRNA mentioned under Example 1, item “Quantitative RT-PCR”. Sequences which decrease the production of WHSC1 or WHSC1L1 gene product in vitro cell-based assays can then be tested for there inhibitory effects on cell growth. Sequences which inhibit cell growth in in vitro cell-based assay can then be tested for their in vivo ability using animals with cancer, e.g., nude mouse xenograft models, to confirm decreased production of WHSC1 or WHSC1L1 gene product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides can also bind each other in the same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is preferably less than 1,000 nucleotides in length. For example, the polynucleotide is less than 500, 200, 100, 75, 50, or 25 nucleotides in length. The isolated polynucleotides of the present invention are useful for forming doublestranded molecules against WHSC1 or WHSC1L1 gene or preparing template DNAs encoding the double-stranded molecules. When the polynucleotides are used for forming double-stranded molecules, the polynucleotide can be longer than 19 nucleotides, preferably longer than 21 nucleotides, and more preferably has a length of between about 19 and 25 nucleotides. Alternatively, the double-stranded molecules of the present invention can be double-stranded molecules, wherein the sense strand is hybridize with antisense strand at the target sequence to form the double-stranded molecule having less than 500, 200, 100, 75, 50 or 25 nucleotides pair in length. Preferably, the double-stranded molecules have between about 19 and about 25 nucleotides pair in length. Further, the sense strand of the double-stranded molecule can preferably include less than 500, 200, 100, 75, 50, 30, 28, 27, 26, 25 nucleotides, more preferably, between about 19 and about 25 nucleotides.

The double-stranded molecule serves as a guide for identifying homologous sequences in mRNA for the RISC complex, when the double-stranded molecule is introduced into cells. The identified target RNA is cleaved and degraded by the nuclease activity of Dicer, through which the double-stranded molecule eventually decreases or inhibits production (expression) of the polypeptide encoded by the RNA. Thus, a double-stranded molecule of the invention can be defined by its ability to generate a single-strand that specifically hybridizes to the mRNA of the WHSC1 or WHSC1L1 gene under stringent conditions. Herein, the portion of the mRNA that hybridizes with the single-strand generated from the double-stranded molecule is referred to as “target sequence” or “target nucleic acid” or “target nucleotide”. In the present invention, nucleotide sequence of the “target sequence” can be shown using not only the RNA sequence of the mRNA, but also the DNA sequence of cDNA synthesized from the mRNA.

The double-stranded molecules of the invention can contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which can be incorporated into the present molecules (WO2003/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C— methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides can be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications can be employed for the double-stranded molecules of the present invention so long as the resulting molecule retains the ability to inhibit the expression of the target gene.

Furthermore, the double-stranded molecules of the present invention can include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type doublestranded molecule composed of a DNA strand (polynucleotide) and an RNA strand (polynucleotide), a chimera type double-stranded molecule containing both DNA and RNA on any or both of the single strands (polynucleotides), or the like can be formed for enhancing stability of the double-stranded molecule.

The hybrid of a DNA strand and an RNA strand can be either where the sense strand is DNA and the antisense strand is RNA, or the opposite so long as it can inhibit expression of the target gene when introduced into a cell expressing the gene. Preferably, the sense strand polynucleotide is DNA and the antisense strand polynucleotide is RNA. Also, the chimera type double-stranded molecule can be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

sense strand:
5′-[---DNA---]-3′
3′-(RNA)-[DNA]-5′:
antisense strand,
sense strand:
5′-(RNA)-[DNA]-3′
3′-(RNA)-[DNA]-5′:
antisense strand,
and
sense strand:
5′-(RNA)-[DNA]-3′
3′-(---RNA---)-5′:
antisense strand.

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type doublestranded molecule, the effect to inhibit expression of the target gene is much higher when the entire antisense strand is RNA (US20050004064).

In the present invention, the double-stranded molecule can form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′ or 5′-[A′]-[B]-[A]-3′, wherein [A] is the sense strand containing a sequence corresponding to a target sequence, [B] is an intervening single-strand and [A′] is the antisense strand containing a complementary sequence to [A]. The target sequence can be selected from among, for example, the nucleotide sequence of SEQ ID NO: 29 or 32 for WHSC1 and the nucleotide sequence of SEQ ID NO: 35 or 38 for WHSC1L1.

The present invention is not limited to these examples, and the target sequence in [A] can be modified sequences from these examples so long as the double-stranded molecule retains the ability to suppress the expression of the targeted WHSC1 or WHSC1L1 gene. The region [A] hybridizes to [A′] to form a loop composed of the region [B]. The intervening single-stranded portion [B], i.e., loop sequence can be preferably 3 to 23 nucleotides in length. The loop sequence, for example, can be selected from among the sequences available from ambion.com/techlib/tb/tb_{—}506.html. Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 Jun., 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

(for target sequence SEQ ID NO: 29)
CAGAUCUACACAGCGGAUA-[B]-UAUCCGCUGUGUAGAUCUG,
(for target sequence SEQ ID NO: 32)
GUUAAUUGGCAUAUGGAAU-[B]-AUUCCAUAUGCCAAUUAAC,
(for target sequence SEQ ID NO: 35)
CUCACAAAUGGGUAUCCAU-[B]-AUGGAUACCCAUUUGUGAG,
and
(for target sequence SEQ ID NO: 38)
GUACUGAAAUUCGGAGACA-[B]-UGUCUCCGAAUUUCAGUAC,

Furthermore, in order to enhance the inhibition activity of the double-stranded molecules, several nucleotides can be added to 3′ end of the sense strand and/or antisense strand of the target sequence, as 3′ overhangs. The preferred examples of nucleotides constituting a 3′ overhang include “t” and “u”, but are not limited thereto. The number of nucleotides to be added is at least 2, generally 2 to 10, preferably 2 to 5. The added nucleotides form single strand at the 3′ end of the sense strand and/or antisense strand of the double-stranded molecule. In cases where double-stranded molecules consists of a single polynucleotide to form a hairpin loop structure, a 3′ overhang sequence can be added to the 3′ end of the single polynucleotide.

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. A specific example for the annealing includes where the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

Alternatively, the double-stranded molecules can be transcribed intracellularly by cloning its coding sequence into a vector containing a regulatory sequence (e.g., a RNA poly III transcription unit from the small nuclear RNA (snRNA) U6 or the human H1 RNA promoter) that directs the expression of the double-stranded molecule in an adequate cell adjacent to the coding sequence. The regulatory sequences flanking the coding sequences of double-stranded molecule can be identical or different, such that their expression can be modulated independently, or in a temporal or spatial manner. Details of vectors capable of producing the double-stranded molecules are described below.

Vector Containing a Double-Stranded Molecule of the Present Invention:

Also included in the present invention are vectors containing one or more of the double-stranded molecules described herein, and a cell containing such a vector. Specifically, the present invention provides the following vector of [1] to [11]:

[1] A vector, encoding a double-stranded molecule that, when introduced into a cell, inhibits in vivo expression of WHSC1 or WHSC1L1 and cell proliferation, such molecules composed of a sense strand and an antisense strand complementary thereto, hybridized to each other to form the double-stranded molecule;

[2] The vector of [1], encoding the double-stranded molecule acts on mRNA, matching a target sequence of SEQ ID NO: 29, 32, 35 or 38;

[3] The vector of [1], wherein the sense strand contains a sequence corresponding to a target sequence of SEQ ID NO: 29, 32, 35 or 38;

[4] The vector of [2] or [3], encoding the double-stranded molecule having a length of less than about 100 nucleotides;

[5] The vector of [4], encoding the double-stranded molecule having a length of less than about 75 nucleotides;

[6] The vector of [5], encoding the double-stranded molecule having a length of less than about 50 nucleotides;

[7] The vector of [6] encoding the double-stranded molecule having a length of less than about 25 nucleotides;

[8] The vector of [7], encoding the double-stranded molecule having a length of between about 19 and about 25 nucleotides;

[9] The vector of any one of [1] to [8], wherein the double-stranded molecule is composed of a single polynucleotide having both the sense and antisense strands linked by an intervening single-strand; and

[10] The vector of [9], encoding the double-stranded molecule having the general formula 5′-[A]-[B]-[A′]-3′ or 5′-[A′]-[B]-[A]-3, wherein [A] is the sense strand containing a sequence corresponding to a target sequence of SEQ ID NO: 29, 32, 35 or 38, [B] is the intervening single-strand composed of 3 to 23 nucleotides, and [A′] is the antisense strand containing a sequence complementary to [A].

[11] A vector including each of a combination of polynucleotide comprising a sense strand nucleic acid and an antisense strand nucleic acid, wherein said sense strand nucleic acid comprises a nucleotide sequence selected from the group consisting of SEQ ID NOs: 29, 32, 35 and 38, and said antisense strand nucleic acid consists of a sequence complementary to the sense strand, wherein the transcripts of said sense strand and said antisense strand hybridize to each other to form a double-stranded molecule, and wherein said vectors inhibits expression of target gene.

A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Accordingly, in one embodiment, the expression vector encodes the nucleic acid sequence of the present invention and is adapted for expression of said nucleic acid sequence. Such vectors of the present invention can be used for producing the present double-stranded molecules, or directly as an active ingredient for treating cancer.

Vectors of the present invention can be produced, for example, by cloning WHSC1 or WHSC1L1 sequence into an expression vector so that regulatory sequences are operatively-linked to WHSC1 or WHSC1L1 sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the doublestranded molecule are utilized to respectively express the sense and anti-sense strands and then forming a double-stranded molecule construct. Furthermore, the cloned sequence can encode a construct having a secondary structure (e.g., hairpin); namely, a single transcript of a vector contains both the sense and complementary antisense sequences of the target gene.

The vectors of the present invention can also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the doublestranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adenoassociated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Method of Inhibiting or Reducing Growth of a Cancer Cell or Treating Cancer Using a Double-Stranded Molecule of the Present Invention:

In present invention, dsRNAs for WHSC1 or WHSC1L1 gene were tested for their ability to inhibit cell growth. The dsRNAs for WHSC1 or WHSC1L1 gene, effectively knocked down the expression of the gene in several cancer cell lines coincided with suppression of cell proliferation (FIG. 4).

Therefore, the present invention provides methods for inhibiting cell growth by inducing dysfunction of WHSC1 or WHSC1L1 gene via inhibiting the expression of WHSC1 or WHSC1L1 gene. WHSC1 or WHSC1L1 gene expression can be inhibited by any of the aforementioned double-stranded molecules of the present invention which specifically target of WHSC1 or WHSC1L1 gene. Examples of cancer cells of which growth can be inhibited by double-stranded molecules against WHSC1 gene or vectors encoding such molecules preferably include, for example, bladder cancer cells, breast cancer cells, cholangiocellular carcinoma cells, CML cells, esophageal cancer cells, HCC cells, NSCLC cells, SCLC cells, osteosarcoma cells, pancreatic cancer cells, prostate cancer cells, renal cell carcinoma cells and soft tissue tumor cells. Examples of cancer cells of which growth can be inhibited by double-stranded molecules against WHSC1L1 gene or vectors encoding such molecules preferably include, for example, bladder cancer cells, breast cancer cells, CML cells, lung cancer cells (e.g., SCLC cells) and lymphoma cells.

The ability of the double-stranded molecules of the present invention and vectors encoding such molecules to inhibit cell growth of cancerous cells indicates that they can be used for methods for treating and/or preventing cancer. For example, the double-stranded molecules against WHSC1 gene or vectors encoding them can be preferably used in treatment and/or prevention for bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor. Also, for example, the double-stranded molecules against WHSC1L1 gene or vectors encoding them can be preferably used in treatment and/or prevention for bladder cancer, breast cancer, CML, lung cancer (e.g., SCLC) and lymphoma. Thus, the present invention provides methods for treating a patient with cancer by administering a double-stranded molecule against WHSC1 or WHSC1L1 gene or a vector expressing the molecule without adverse effect because WHSC1 or WHSC1L1 gene is minimally expressed in normal organs (FIG. 1, 2, 3).

Specifically, the present invention provides the following methods of [1] to [33]:

[1] A method for inhibiting a growth of cancer cell, and treating and/or preventing cancer, wherein the cancer cell or the cancer expresses a WHSC1 and/or WHSC1L1 gene, which method includes the step of administering at least one isolated doublestranded molecule inhibiting the expression of WHSC1 or WHSC1L1 gene in a cell over-expressing the gene and the cell proliferation, wherein the double-stranded molecule is composed of a sense strand and an antisense strand complementary thereto, hybridized to each other to form the double-stranded molecule;

[2] A method of treating and/or preventing cancer in a subject comprising administering to the subject a pharmaceutically effective amount of a double-stranded molecule against a WHSC1 or WHSC1L1 gene or a vector encoding thereof, and a pharmaceutically acceptable carrier, wherein the double-stranded molecule inhibits the cell proliferation as well as the expression of the WHSC1 or WHSC1L1 gene when introduced into a cell expressing the WHSC1 or WHSC1L1 gene;

[3] The method of [1] or [2], wherein the double-stranded molecule acts at mRNA which matches a target sequence of SEQ ID NO: 29, 32, 35 or 38;

[4] The method of [1] or [2], wherein the sense strand contains a sequence corresponding to a target sequence of SEQ ID NO: 29, 32, 35 or 38;

[5] The method of any one of [1] to [4], wherein the cancer is bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma;

[6] The method of any one of [1] to [5], wherein the double-stranded molecule has a length of less than about 100 nucleotides;

[7] The method of [6], wherein the double-stranded molecule has a length of less than about 75 nucleotides;

[8] The method of [7], wherein the double-stranded molecule has a length of less than about 50 nucleotides;

[9] The method of [8], wherein the double-stranded molecule has a length of less than about 25 nucleotides;

[10] The method of [9], wherein the double-stranded molecule has a length of between about 19 and about 25 nucleotides in length;

[11] The method of any one of [1] to [10], wherein the double-stranded molecule is composed of a single polynucleotide containing both the sense strand and the antisense strand linked by an intervening single-strand;

[12] The method of Mt wherein the double-stranded molecule has the general formula 5′-[A]-[B]-[A′]-3′ or 5′-[A′]-[B]-[A]-3, wherein [A] is the sense strand containing a sequence corresponding to a target sequence of SEQ ID NO: 29, 32, 35 or 38, [B] is the intervening single strand composed of 3 to 23 nucleotides, and [A′] is the antisense strand containing a sequence complementary to [A];

[13] The method of any one of [1] to [12], wherein the double-stranded molecule is an RNA;

[14] The method of any one of [1] to [12], wherein the double-stranded molecule contains both DNA and RNA;

[15] The method of [14], wherein the double-stranded molecule is a hybrid of a DNA polynucleotide and an RNA polynucleotide;

[16] The method of [15] wherein the sense and antisense strand polynucleotides are composed of DNA and RNA, respectively;

[17] The method of [14], wherein the double-stranded molecule is a chimera of DNA and RNA;

[18] The method of [17], wherein a region flanking to the 3′-end of the antisense strand, or both of a region flanking to the 5′-end of sense strand and a region flanking to the 3′-end of antisense strand are composed of RNA;

[19] The method of [18], wherein the flanking region is composed of 9 to 13 nucleotides;

[20] The method of any one of [1] to [19], wherein the double-stranded molecule contains one or two 3′ overhangs;

[21] The method of any one of [1] to [20], wherein the double-stranded molecule or the vector is contained in a composition which includes, in addition to the molecule, a transfection-enhancing agent and pharmaceutically acceptable carrier.

[22] The method of [1], wherein the double-stranded molecule is encoded by a vector;

[23] The method of [22], wherein the double-stranded molecule encoded by the vector acts at mRNA which matches a target sequence of SEQ ID NO: 29, 32, 35 or 38;

[24] The method of [23], wherein the sense strand of the double-stranded molecule encoded by the vector contains a sequence corresponding to a target sequence of SEQ ID NO: 29, 32, 35 or 38;

[25] The method of any one of [22] to [24], wherein the cancer is bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma;

[26] The method of any one of [22] to [25], wherein the double-stranded molecule encoded by the vector has a length of less than about 100 nucleotides;

[27] The method of [26], wherein the double-stranded molecule encoded by the vector has a length of less than about 75 nucleotides;

[28] The method of [27], wherein the double-stranded molecule encoded by the vector has a length of less than about 50 nucleotides;

[29] The method of [28], wherein the double-stranded molecule encoded by the vector has a length of less than about 25 nucleotides;

[30] The method of [29], wherein the double-stranded molecule encoded by the vector has a length of between about 19 and about 25 nucleotides in length;

[31] The method of any one of [22] to [30], wherein the double-stranded molecule encoded by the vector is composed of a single polynucleotide containing both the sense strand and the antisense strand linked by an intervening single-strand;

[32] The method of [31], wherein the double-stranded molecule encoded by the vector has the general formula 5′-[A]-[B]-[A′]-3′ or 5′-[A′]-[B]-[A]-3, wherein [A] is the sense strand containing a sequence corresponding to a target sequence of SEQ ID NO: 29, 32, 35 or 38, [B] is a intervening single-strand is composed of 3 to 23 nucleotides, and [A′] is the antisense strand containing a sequence complementary to [A]; and

[33] The method of any one of [22] to [32], wherein the double-stranded molecule encoded by the vector is contained in a composition which includes, in addition to the molecule, a transfection-enhancing agent and pharmaceutically acceptable carrier.

In addition, in another embodiment, [1] a method for a purpose selected from the group consisting of:

(a) inhibiting a growth of cancer cell,

(b) treating cancer, and

(b) preventing cancer,

wherein the cancer cell or the cancer expresses either or both of a WHSC1 and WHSC1L1 gene, which method includes the step of administering at least one isolated double-stranded molecule inhibiting the expression of WHSC1 or WHSC1L1 gene in a cell over-expressing the gene and the cell proliferation, wherein the double-stranded molecule is composed of a sense strand and an antisense strand complementary thereto, hybridized to each other to form the double-stranded molecule is provided.

In addition, in another embodiment, [2] a method of either or both of treating and preventing cancer in a subject, comprising administering to the subject a pharmaceutically effective amount of a double-stranded molecule against a WHSC1 or WHSC1L1 gene, or a vector encoding thereof, and a pharmaceutically acceptable carrier, wherein the double-stranded molecule inhibits the cell proliferation and the expression of the WHSC1 or WHSC1L1 gene when introduced into a cell expressing the WHSC1 or WHSC1L1 gene is also provided.

The method of the present invention will be described in more detail below.

The growth of cells expressing WHSC1 and/or WHSC1L1 genes can be inhibited by contacting the cells with a double-stranded molecule against WHSC1 or WHSC1L1 gene, a vector expressing the molecule or a composition containing the same. The cell can be further contacted with a transfection agent. Suitable transfection agents are known in the art. The phrase “inhibition of cell growth” indicates that the cell proliferates at a lower rate or has decreased viability as compared to a cell not exposed to the molecule. Cell growth can be measured by methods known in the art, e.g., using the MTT cell proliferation assay.

The growth of any kind of cell can be suppressed according to the present method so long as the cell expresses or over-expresses the target gene of the double-stranded molecule of the present invention. Exemplary cells that over-express WHSC1 gene include, for example, bladder cancer cells, breast cancer cells, cholangiocellular carcinoma cells, CML, esophageal cancer cells, HCC cells, NSCLC cells, SCLC cells, osteosarcoma cells, pancreatic cancer cells, prostate cancer cells, renal cell carcinoma cells and soft tissue tumor. Exemplary cells that ever-express WHSC1L1 gene include, for example, bladder cancer cells, breast cancer cells, CML cells, lung cancer cells (e.g., SCLC cells) and lymphoma cells.

Thus, patients suffering from or at risk of developing disease related to WHSC1 and/or WHSC1L1 overexpression can be treated by administering the presents at least one double-stranded molecule of the present invention, at least one vector expressing the molecule or composition containing the molecule. For example, cancer patients can be treated according to the present methods. The type of cancer can be identified by standard methods according to the particular type of tumor to be diagnosed. More preferably, patients treated by the methods of the present invention are selected by detecting the expression of WHSC1 or WHSC1L1 gene in a biopsy specimen or sample from the patient by RT-PCR or immunoassay. Preferably, before the treatment of the present invention, the biopsy specimen or sample from the subject is confirmed for WHSC1 or WHSC1L1 gene over-expression by methods known in the art, for example, methods described in the item of “A method for diagnosing cancer” such as immunohistochemical analysis or RT-PCR.

For inhibiting cell growth, a double-stranded molecule of the present invention can be directly introduced into the cells in a form to achieve binding of the molecule with corresponding mRNA transcripts. Alternatively, as described above, a DNA encoding the double-stranded molecule can be introduced into cells as a vector. For introducing the double-stranded molecules or vectors into the cells, a transfection-enhancing agent, such as FuGENE (Roche diagnostics), Lipofectamine 2000 (Invitrogen), Oligofectamine (Invitrogen), and Nucleofector (Wako pure Chemical), can be employed.

A treatment is deemed “efficacious” if it leads to a clinical benefit such as, reduction in expression of WHSC1 or WHSC1L1 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecule of the present invention degrades the WHSC1 or WHSC1L1 mRNA in substoichiometric amounts. Without wishing to be bound by any theory, it is believed that the double-stranded molecule of the invention causes degradation of the target mRNA in a catalytic manner. Thus, compared to standard cancer therapies, significantly less a double-stranded molecule needs to be delivered at or near the site of cancer to exert therapeutic effect.

One skilled in the art can readily determine an effective amount of the doublestranded molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the invention is an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance can be readily and routinely determined by one of skill in the art.

The present methods can be used to inhibit the growth or metastasis of cancer expressing WHSC1 and/or WHSC1L1. For example, cancers expressing WHSC1 include bladder cancer, breast cancer, cholangiocellular carcinoma, CML, esophageal cancer, HCC, NSCLC, SCLC, osteosarcoma, pancreatic cancer, prostate cancer, renal cell carcinoma and soft tissue tumor. For example, cancers expressing WHSC1L1 include bladder cancer, breast cancer, CML, lung cancer (e.g., SCLC) and lymphoma. In particular, a double-stranded molecule containing a nucleotide sequence corresponding to a target sequence of WHSC1 (preferably, SEQ ID NO: 29 or 32) or a target sequence of WHSC1L1 (preferably, SEQ ID NO: 35 or 38) is particularly preferred for the treatment of cancer.

For treating cancer, the double-stranded molecule of the present invention can also be administered to a subject in combination with a pharmaceutical substance different from the double-stranded molecule of the present invention. Alternatively, the doublestranded molecule of the present invention can be administered to a subject in combination with another therapeutic method designed to treat cancer. For example, the double-stranded molecule of the present invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule of the present invention can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as lung tumor tissue, and can also increase the blood half-life of the double-stranded molecule. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule include a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60 degrees C.

Vectors expressing a double-stranded molecule of the invention are discussed above. Such vectors expressing at least one double-stranded molecule of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the present invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intraarterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example by a catheter or other placement device (e.g., a suppository or an implant including a porous, non-porous, or gelatinous material); and inhalation. It is preferred that injections or infusions of the double-stranded molecule or vector be given at or near the site of cancer.

The double-stranded molecule of the present invention can be administered in a single dose or in multiple doses. Where the administration of the double-stranded molecule of the invention is by infusion, the infusion can be a single sustained dose or can be delivered by multiple infusions. Injection of the substance directly into the tissue is at or near the site of cancer preferred. Multiple injections of the substance into the tissue at or near the site of cancer are particularly preferred.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen includes multiple administrations, it is understood that the effective amount of a double-stranded molecule administered to the subject can include the total amount of a double-stranded molecule administered over the entire dosage regimen.

In the present invention, a cancer overexpressing WHSC1 or WHSC1L1 gene can be treated with at least one active ingredient selected from the group consisting of:

(a) a double-stranded molecule of the present invention,

(b) DNA encoding said double-stranded molecule, and

(c) a vector encoding said double-stranded molecule.

The cancer includes, but is not limited to, bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma. Accordingly, prior to the administration of the double-stranded molecule of the present invention as active ingredient, it is preferable to confirm whether the expression level of WHSC1 or WHSC1L1 in the cancer cells or tissues to be treated is enhanced as compared with normal cells of the same organ. Thus, in one embodiment, the present invention provides a method for treating a cancer overexpressing WHSC1 or WHSC1L1 gene, which method can include the steps of:

i) determining the expression level of WHSC1 or WHSC1L1 gene in cancer cells or tissue(s) obtained from a subject with the cancer to be treated;

ii) comparing the expression level of WHSC1 or WHSC1L1 with normal control; and

iii) administrating at least one component selected from the group consisting of

(a) a double-stranded molecule of the present invention,

(b) DNA encoding said double-stranded molecule, and

(c) a vector encoding said double-stranded molecule,

to a subject with a cancer overexpressing WHSC1 or WHSC1L1 gene compared with normal control. Alternatively, the present invention also provides a pharmaceutical composition comprising at least one component selected from the group consisting of:

(a) a double-stranded molecule of the present invention,

(b) DNA encoding said double-stranded molecule, and

(c) a vector encoding said double-stranded molecule,

for use in administrating to a subject with a cancer overexpressing WHSC1 or WHSC1L1 gene. In other words, the present invention further provides a method for identifying a subject to be treated with:

(a) a double-stranded molecule of the present invention,

(b) DNA encoding said double-stranded molecule, or

(c) a vector encoding said double-stranded molecule,

which method can include the step of determining an expression level of WHSC1 or WHSC1L1 in subject-derived cancer cells or tissue(s), wherein an increase of the level compared to a normal control level of the gene indicates that the subject has cancer which can be treated with a double-stranded molecule of the present invention.

The method of treating a cancer of the present invention will be described in more detail below.

A subject to be treated by the present method is preferably a mammal. Exemplary mammals include, but are not limited to, e.g., human, non-human primate, mouse, rat, dog, cat, horse, and cow.

According to the present invention, the expression level of WHSC1 or WHSC1L1 gene in cancer cells or tissues obtained from a subject is determined. The expression level can be determined at the transcription product (mRNA) level, using methods known in the art. For example, hybridization methods (e.g., Northern hybridization), a chip or an array, probes, RT-PCR can be used to determine the transcription product level of WHSC1 or WHSC1L1 gene.

Alternatively, the translation product (polypeptide or protein) can be detected for the treatment of the present invention. For example, the quantity of observed protein can be determined.

As another method to detect the expression level of WHSC1 or WHSC1L1 gene based on its translation product, the intensity of staining can be measured via immunohistochemical analysis using an antibody against the WHSC1 or WHSC1L1 protein. Namely, in this measurement, strong staining indicates increased presence/level of the protein and, at the same time, high expression level of WHSC1 or WHSC1L1 gene.

Methods for detecting or measuring the WHSC1 or WHSCL1 polypeptide, and the polynucleotide encoding thereof can be exemplified as described above (A method for Diagnosing cancer).

Compositions Containing a Double-Stranded Molecule of the Present Invention:

In addition to the above, the present invention also provides pharmaceutical composition that include the double-stranded molecule of the present invention or the vector coding for the molecule.

Specifically, the present invention provides the following compositions [1] to [33]:

[1] A composition for inhibiting a growth of cancer cell and treating a cancer, wherein the cancer cell and the cancer expresses a WHSC1 or WHSC1L1 gene, including isolated double-stranded molecule inhibiting the expression of WHSC1 or WHSC1L1 and the cell proliferation, which molecule is composed of a sense strand and an antisense strand complementary thereto, hybridized to each other to form the double-stranded molecule;

[2] A composition for treating and/or preventing cancer, which comprises a pharmaceutically effective amount of a double-stranded molecule against a WHSC1 or WHSC1L1 gene or a vector encoding said double-stranded molecule, and a pharmaceutically acceptable carrier, wherein the double-stranded molecule inhibits cell proliferation as well as expression of the WHSC1 or WHSC1L1 gene when introduced into a cell expressing the WHSC1 or WHSC1L1 gene;

[3] The composition of [1] or [2], wherein the double-stranded molecule acts at mRNA which matches a target sequence of SEQ ID NO: 29, 32, 35 or 38;

[3] The composition of [1] or [2], wherein the double-stranded molecule, wherein the sense strand contains a sequence corresponding to a target sequence of SEQ ID NO: 29, 32, 35 or 38;

[5] The composition of [1], wherein the cancer is bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma;

[6] The composition of any one of [1] to [5], wherein the double-stranded molecule has a length of less than about 100 nucleotides;

[7] The composition of [6], wherein the double-stranded molecule has a length of less than about 75 nucleotides;

[8] The composition of [7], wherein the double-stranded molecule has a length of less than about 50 nucleotides;

[9] The composition of [8], wherein the double-stranded molecule has a length of less than about 25 nucleotides;

[10] The composition of [9], wherein the double-stranded molecule has a length of between about 19 and about 25 nucleotides;

[11] The composition of any one of [1] to [10], wherein the double-stranded molecule is composed of a single polynucleotide containing the sense strand and the antisense strand linked by an intervening single-strand;

[12] The composition of Mt wherein the double-stranded molecule has the general formula 5′-[A]-[B]-[A′]-3′ or 5′-[A′]-[B]-[A]-3′, wherein [A] is the sense strand sequence contains a sequence corresponding to a target sequence of SEQ ID NO: 29, 32, 35 or 38, [B] is the intervening single-strand consisting of 3 to 23 nucleotides, and [A′] is the antisense strand contains a sequence complementary to [A];

[13] The composition of any one of [1] to [12], wherein the double-stranded molecule is an RNA;

[14] The composition of any one of [1] to [12], wherein the double-stranded molecule is composed of DNA and RNA;

[15] The composition of [14], wherein the double-stranded molecule is a hybrid of a DNA polynucleotide and an RNA polynucleotide;

[16] The composition of [15], wherein the sense and antisense strand polynucleotides are composed of DNA and RNA, respectively;

[17] The composition of [14], wherein the double-stranded molecule is a chimera of DNA and RNA;

[18] The composition of [15], wherein a region flanking to the 3′-end of the antisense strand, or both of a region flanking to the 5′-end of sense strand and a region flanking to the 3′-end of antisense strand are composed of RNA;

[19] The composition of [18], wherein the flanking region is composed of 9 to 13 nucleotides;

[20] The composition of any one of [1] to [19], wherein the double-stranded molecule contains one or two 3′ overhangs;

[21] The composition of any one of [1] to [20], wherein the composition includes a transfection-enhancing agent and a pharmaceutically acceptable carrier;

[22] The composition of any one of [1] to [21], wherein the double-stranded molecule is encoded by a vector;

[23] The composition of [22], wherein the double-stranded molecule encoded by the vector acts at mRNA which matches a target sequence of SEQ ID NO: 29, 32, 35 or 38;

[24] The composition of [22], wherein the sense strand of the double-stranded molecule encoded by the vector contains a sequence corresponding to a target sequence of SEQ ID NO: 29, 32, 35 or 38;

[25] The composition of any one of [22] to [24], wherein the cancer is bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma;

[26] The composition of any one of [22] to [25], wherein the double-stranded molecule encoded by the vector has a length of less than about 100 nucleotides;

[27] The composition of [26], wherein the double-stranded molecule encoded by the vector has a length of less than about 75 nucleotides;

[28] The composition of [27], wherein the double-stranded molecule encoded by the vector has a length of less than about 50 nucleotides;

[29] The composition of [28], wherein the double-stranded molecule encoded by the vector has a length of less than about 25 nucleotides;

[30] The composition of [29], wherein the double-stranded molecule encoded by the vector has a length of between about 19 and about 25 nucleotides in length;

[31] The composition of any one of [22] to [29], wherein the double-stranded molecule encoded by the vector is composed of a single polynucleotide containing both the sense strand and the antisense strand linked by an intervening single-strand;

[32] The composition of [31], wherein the double-stranded molecule has the general formula 5′-[A]-[B]-[A′]-3′ or 5′-[A′]-[B]-[A]-3′, wherein [A] is the sense strand containing a sequence corresponding to a target sequence of SEQ ID NO: 29, 32, 35 or 38, [B] is a intervening single-strand composed of 3 to 23 nucleotides, and [A′] is the antisense strand containing a sequence complementary to [A]; and

[33] The composition of any one of [22] to [32], wherein the composition includes a transfection-enhancing agent and a pharmaceutically acceptable carrier.

In addition, in another embodiment, [2] a composition for either or both of treating and preventing cancer, which comprises a pharmaceutically effective amount of a double-stranded molecule against a WHSC1 or WHSC1L1 gene or a vector encoding said double-stranded molecule, and a pharmaceutically acceptable carrier, wherein the double-stranded molecule inhibits cell proliferation and the expression of the WHSC1 or WHSC1L1 gene when introduced into a cell expressing the WHSC1 or WHSC1L1 gene is provided.

Suitable compositions of the present invention are described in additional detail below.

The double-stranded molecule of the present invention is preferably formulated as pharmaceutical compositions prior to administering to a subject, according to techniques known in the art. Pharmaceutical composition of the present invention is characterized as being at least sterile and pyrogen-free. As used herein, “pharmaceutical composition” includes formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical composition contains the double-stranded molecule of the present invention or vector encoding the molecule (e.g., 0.1 to 90% by weight), or a physiologically acceptable salt of the molecule, mixed with a physiologically or pharmaceutically acceptable carrier medium. Preferred physiologically or pharmaceutically acceptable carrier media are water, buffered water, normal saline, 0.4% saline, 0.3% glycine, hyaluronic acid and the like.

Moreover, the double-stranded molecule of the present invention can be contained in liposomes in the present composition. See under the item of “Methods of treating cancer using the double-stranded molecule” for details of liposomes.

Pharmaceutical compositions of the present invention can also include conventional pharmaceutical excipients and/or additives. Suitable pharmaceutical excipients include, for example, stabilizers, antioxidants, osmolality adjusting agents, buffers, and pH adjusting agents. Suitable additives include, for example, physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25-75%, of one or more double-stranded molecule of the invention. A pharmaceutical composition for aerosol (inhalational) administration can include 0.01-20% by weight, preferably 1-10% by weight, of one or more double-stranded molecule of the invention encapsulated in a liposome as described above, and propellant. A carrier can also be included as desired; e.g., lecithin for intranasal delivery.

In addition to the above, the present composition can contain other pharmaceutical active ingredients so long as they do not inhibit the in vivo function of the doublestranded molecules of the present invention. For example, the composition of the present invention can contain chemotherapeutic agents conventionally used for treating cancers.

In another embodiment, the present invention also provides the use of the doublestranded molecule of the present invention in manufacturing a pharmaceutical composition for treating and/or preventing cancer characterized by the expression of WHSC1 or WHSC1L1 gene. For example, the present invention relates to a use of double-stranded molecule inhibiting the expression of WHSC1 or WHSC1L1 gene in a cell, which molecule includes a sense strand and an antisense strand complementary thereto, hybridized to each other to form the double-stranded molecule and target to a sequence of SEQ ID NO: 29, 32, 35 or 38, for manufacturing a pharmaceutical composition for treating cancer expressing WHSC1 or WHSC1L1 gene.

Alternatively, the present invention further provides a method or process for manufacturing a pharmaceutical composition for treating and/or preventing cancer characterized by the expression of WHSC1 or WHSC1L1 gene, wherein the method or process includes a step for formulating a pharmaceutically or physiologically acceptable carrier with a double-stranded molecule inhibiting the expression of WHSC1 or WHSC1L1 gene in a cell, which over-expresses the gene, which molecule includes a sense strand and an antisense strand complementary thereto, hybridized to each other to form the double-stranded molecule and target to a sequence of SEQ ID NO: 29, 32, or 38 as active ingredients.

Alternatively, the present invention further provides the double-stranded nucleic acid molecules of the present invention for use in treating a cancer expressing the WHSC1 or WHSC1L1 gene.

In another embodiment, the present invention also provides a method or process for manufacturing a pharmaceutical composition for treating and/or preventing cancer characterized by the expression of WHSC1 or WHSC1L1 gene, wherein the method or process includes a step for admixing an active ingredient with a pharmaceutically or physiologically acceptable carrier, wherein the active ingredient is a double-stranded molecule inhibiting the expression of WHSC1 or WHSC1L1 gene in a cell, which over-expresses the gene, which molecule includes a sense strand and an antisense strand complementary thereto, hybridized to each other to form the double-stranded molecule and targets to a sequence of SEQ ID NO: 29, 32, 35 or 38.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The invention will be further described in the following examples, which do not limit the scope of the invention described in the claims.

### Example 1

### General Methods

Bladder Tissue Samples and RNA Preparation

120 surgical specimens of primary urothelial carcinoma were collected, either at cystectomy or transurethral resection of bladder tumor (TURBT), and snap frozen in liquid nitrogen. 22 specimens of normal bladder urothelial tissue were collected from areas of macroscopically normal bladder urothelium in patients with no evidence of malignancy. Five sequential sections of 7 micro-m thickness were cut from each tissue and stained using Histogene™ staining solution (Arcturus, Calif., USA) following the manufacturer's protocol, and assessed for cellularity and tumor grade by an independent consultant urohistopathologist. Slides were then transferred for microdissection using a Pix Cell II laser capture microscope (Arcturus, Calif., USA). This technique employs a low-power infrared laser to melt a thermoplastic film over the cells of interest, to which the cells become attached. Additionally, the sections were graded according to the degree of inflammatory cell infiltration (low, moderate and severe). Samples showing significant inflammatory cell infiltration were excluded (Wallard M et al. Br J Cancer 94: 569-577, 2006).

Approximately 10,000 cells were microdissected from both stromal and epithelial/tumor compartments in each tissue. RNA was extracted using an RNeasy Micro Kit (QIAGEN, Crawley, UK). Areas of cancer or stroma containing significant inflammatory areas of tumor or stroma containing significant inflammatory cell infiltration were avoided to prevent contamination (Wallard M et al. Br J Cancer 94: 569-577, 2006). Total RNA was reverse transcribed, and qRT-PCR was performed as described below. Given the low yield of RNA from such small samples, NanoDrop™ quantification was not performed, but correction for the endogenous 18S CT value was used as an accurate measure of the amount of intact starting RNA. To validate the accuracy of microdissection, primers and probes for Vimentin and Uroplakin were sourced and qRT-PCR performed according to the manufacturer's instructions (Assays on demand, Applied Biosystems, Warrington, UK). Vimentin is primarily expressed in messenchymally derived cells, and was used as a stromal marker. Uroplakin is a marker of urothelial differentiation and is preserved in up to 90% of epithelially derived tumors (Olsburgh J et al. J Pathol 199: 41-49, 2003). Use of tissues in the examples was approved by Cambridge shire Local Research Ethics Committee (Ref 03/018).

Lung Tissue Samples for Tissue Microarray

Primary non-SCLC(NSCLC) tissue samples as well as their corresponding normal tissues adjacent to resection margins from patients having no anticancer treatment before tumor resection had been obtained earlier with informed consent (Kato T, et al. Cancer Res 2005; 65:5638-46., Kikuchi T, et al. Oncogene 2003; 22:2192-205., Taniwaki M, et al. Int J Oncol 2006; 29:567-75.). All tumors were staged on the basis of the pathologic tumor-node-metastasis classification of the International Union Against Cancer. Formalin-fixed primary lung tumors and adjacent normal lung tissue samples used for immunostaining on tissue microarrays had been obtained from 328 patients undergoing curative surgery at Saitama Cancer Center (Saitama, Japan) (Ishikawa N, et al. Clin Cancer Res 2004; 10:8363-70., Ishikawa N, et al. Cancer Res 2007; 67:11601-11.). To be eligible for this study, tumor samples were selected from patients who fulfilled all of the following criteria: (a) patients suffered primary NSCLC with histologically confirmed stage (only pT1 to pT3, pN0 to pN2, and pM0); (b) patients underwent curative surgery, but did not receive any preoperative treatment; (c) among them, NSCLC patients with positive lymph node metastasis (pN1, pN2) were treated with platinum-based adjuvant chemotherapies after surgical resection, whereas patients with pN0 did not receive adjuvant chemotherapies; and (d) patients whose clinical follow-up data were available. This study and the use of all clinical materials mentioned were approved by individual institutional ethics committees.

Cell Culture

All cell lines were grown in monolayers in appropriate media: Eagle's minimal essential medium (EMEM) for IMR-90, 253J, 253J-BV, HT1197, HT1376, J82, SCaBER, UMUC3 bladder cancer cells and SBC5 small cell lung cancer cells; RPMI1640 medium for 5637 bladder cancer cells and A549, NCI-H2170 and LC319 non-small cell lung cancer cells, and SNU475 hepatocellular cancer cells; Dulbecco's modified Eagle's medium (DMEM) for EJ28 bladder cancer cells, RERF-LC-AI non-small cell lung cancer cells, HepG2 hepatocellular cancer cells and 293T cells; McCoy's 5A medium for RT4 and T24 bladder cancer cells and HCT116 colorectal cancer cells; Leibovitz's L-15 for SW780 and SW480 cells supplemented with 10% fetal bovine serum and 1% antibiotic/antimycotic solution (Sigma). LoVo cells were cultured in Ham's F-12 medium supplemented with 20% fetal bovine serum and 1% antibiotic/antimycotic solution (Sigma). SAEC cells were maintained in small airway epithelial cell basal medium supplemented with 52 micro-g/ml bovine pituitary extract, 0.5 ng/ml human recombinant EGF, 0.5 micro-g/ml hydrocortisone, 0.5 micro-g/ml epinephrine, 10 micro-g/ml transferrin, 5 micro-g/ml insulin, 0.1 ng/ml retinoic acid (RA), 6.5 ng/ml triiodothyronine, 50 micro-g/ml Gentamicin/Amphotericin-B (GA-1000) and 50 micro-g/ml fatty acid-free bovine serum albumin (BSA). All cells were maintained at 37 degrees C. in humid air with 5% CO₂, (IMR-90, SAEC, 5637, 253J, 253J-BV, EJ28, HT1197, HT1376, J82, RT4, SCaBER, T24, UMUC3, A549, H2170, LC319, RERF-LC-AI, SBC5, 293T, HepG2, SNU475, Huh7 and LoVo) or without CO₂ (SW780 and SW480). Cells were transfected with FuGENE6 (ROCHE, Basel, Switzerland) according to manufacturer's protocols.

Expression Profiling in Cancer Using cDNA Microarrays

The present inventors established a genome-wide cDNA microarray with 36,864 cDNAs selected from the UniGene database of the National Center for Biotechnology Information (NCBI). This microarray system was constructed essentially as described previously (Kikuchi T et al. Oncogene 22: 2192-2205, 2003, Kitahara O et al. Cancer Res 61: 3544-3549, 2001, Nakamura T et al. Oncogene 23: 2385-2400, 2004). Briefly, the cDNAs were amplified by RT-PCR using poly (A)+ RNAs isolated from various human organs as templates; the lengths of the amplicons ranged from 200 to 1,100 bp, without any repetitive or poly (A) sequences. Many types of tumor and corresponding non-neoplastic tissues were prepared in 8 micro-m, as described previously (Kitahara O et al. Cancer Res 61: 3544-3549, 2001). A total of 30,000-40,000 cancer or non-cancerous cells were collected selectively using the EZ cut system (SL Microtest GmbH, Germany) according to the manufacturer's protocol. Extraction of total RNA, T7-based amplification, and labeling of probes were performed as described previously (Kitahara O et al. Cancer Res 61: 3544-3549, 2001). A measure of 2.5 micro-g aliquots of twice-amplified RNA (aRNA) from each cancerous and non cancerous tissue was then labeled, respectively, with Cy3-dCTP or Cy5-dCTP.

Quantitative Real-Time PCR

As described previously, the present inventors prepared 121 bladder cancer and 24 normal bladder tissues in Cambridge Addenbrooke's Hospital. For quantitative RT-PCR reactions, specific primers for all human GAPDH (housekeeping gene), SDH (housekeeping gene), WHSC1 and WHSC1L1 were designed (primer sequences in Table 1). PCR reactions were performed using the ABI prism 7700 Sequence Detection System (Applied Biosystems, Warrington, UK) following the manufacture's protocol. 50% SYBR GREEN universal PCR Master Mix without UNG (Applied Biosystems, Warrington, UK), 50 nano M each of the forward and reverse primers and 2 micro-1 of reverse transcriptional cDNA were applied. Amplification conditions were firstly 5 min at 95 degrees C. and then 45 cycles each consisting of 10 sec at 95 degrees C., 1 min at 55 degrees C. and 10 sec at 72 degrees C. After this, samples were incubated for 15 sec at 95 degrees C., 1 min at 65 degrees C. to draw the melting curve, and cooled to 50 degrees C. for 10 sec. Reaction conditions for target gene amplification were as described above and 5 nano g of reverse transcribed RNA was used in each reaction.

TABLE 1
Primer sequences for quantitative RT-PCR

| Gene name | Primer sequence | SEQ ID NO. |
|---|---|---|
| GAPDH (housekeeping gene)-f | 5′ GCAAATTCCATGGCACCGTC 3′ | 5 |
| GAPDH (housekeeping gene)-r | 5′ TCGCCCCACTTGATTTTGG 3′ | 6 |
| SDH (housekeeping gene)-f | 5′ TGGGAACAAGAGGGCATCTG 3′ | 7 |
| SDH (housekeeping gene)-r | 5′ CCACCACTGCATCAAATTCATG 3′ | 8 |
| WHSC1-f1 | 5′ TCGAAGCAGCTCTTGTGTCTAAG 3′ | 9 |
| WHSC1-r1 | 5′ TTTGGACCACACCAAATCACCAAC 3′ | 10 |
| WHSC1-f2 | 5′ AATATGACTCCTTGCTGGAGCAGG 3′ | 11 |
| WHSC1-r2 | 5′ ATTTCAACAGGTGGTCTTTGTCTC 3′ | 12 |
| WHSC1L1-f1 | 5′ AGAACGTGCTCAGTGGGATATTGG 3′ | 13 |
| WHSC1L1-r1 | 5′ TGCTTGGGATAAAGCCTCTTCAGG 3′ | 14 |
| WHSC1L1-f2 | 5′ CAAGCCAGCAATCACTCTGAGAAAC 3′ | 15 |
| WHSC1L1-r2 | 5′ TATACTGTTCTATTCTTTCTTCTCG 3′ | 16 |

Immunohistochemical Staining

Sections of human bladder tissues were stained by VECTASTAIN (registered trademark) ABC KIT (VECTOR LABORATORIES, CA, USA). Briefly, endogenous peroxidase activity of xylene-deparaffinized and dehydrated sections was inhibited by treatment with 0.3% H₂O₂/methanol. Nonspecific binding was blocked by incubating sections with 3% BSA in a humidified chamber for 30 min at ambient temperature followed by overnight incubation at 4 degrees C. with a 1:500 dilution of rabbit polyclonal anti-WHSC1 (HPA015801, SIGMA-ALDRICH, St. Louis, Mo., USA) antibody. The sections were washed twice with PBS (−), incubated with a 1:500 dilution of goat anti-rabbit biotinylated IgG and a 1:500 dilution of goat anti-mouse biotinylated IgG in PBS (−) containing 1% BSA for 30 min at ambient temperature, and then incubated with ABC reagent for 30 min. Specific immunostaining was visualized by 3,3′-diaminobenzidine. Slides were dehydrated through graded alcohol and xylene washing, and mounted on cover slips. Hematoxylin was used for nuclear counterstaining.

The expression patterns of WHSC1 in bladder and lung tumors, and normal human tissues were examined by immunohistochemistry as described previously (Unoki M, et al. Br J Cancer 2009; 101:98-105.). Briefly, slides of paraffin-embedded bladder tumor specimens and normal human tissues were processed under high pressure (125 degrees C., 30 s) in antigen-retrieval solution, high pH 9 (S2367, Dako Cytomation, Carpinteria, Calif., USA), treated with peroxidase blocking regent, and then treated with protein blocking regent (K130, X0909, Dako Cytomation). Tissue sections were incubated with the rabbit anti-WHSC1 polyclonal antibody (HPA015801, 1:25, SIGMA-ALDRICH, St. Lois, Mo.), the rabbit anti-WHSC1L1 polyclonal antibody (HPA005659, 1:25, SIGMA-ALDRICH, St. Louis, Mo.) or normal rabbit IgG (1:25, Santa Cruz, Santa Cruz, Calif., USA) followed by HRP-conjugated secondary antibody (Dako Cytomation). Antigen was visualized with substrate chromogen (Dako liquid DAB chromogen; Dako Cytomation). Finally, tissue specimens were stained with Mayer's haematoxylin (Muto pure chemicals Ltd, Tokyo, Japan) for 20 s to discriminate the nucleus from the cytoplasm.

siRNA Transfection

siRNA oligonucleotide duplexes were purchased from SIGMA Genosys for targeting the human WHSC1 and WHSC1L1 transcripts. siEGFP and siNegative control (siNC), which is a mixture of three different oligonucleotide duplexes, were used as control siRNAs. The siRNA sequences are described in Table 2. siRNA duplexes (100 nM final concentration) were transfected into bladder and lung cancer cell lines with Lipofectamine 2000 (Invitrogen) for 72 hrs, and cell viability was examined using the Cell Counting Kit-8 (Dojindo, Kumamoto, Japan).

TABLE 2
siRNA sequences

| siRNA name | | Sequence | SEQ IN NO. |
|---|---|---|---|
| siEGFP | | Sense: 5′ GCAGCACGACUUCUUCAAGTT 3′ | 17 |
| | | Antisense: 5′ CUUGAAGAAGUCGUGCUGCTT 3′ | 18 |
| siFFLuc | | Sense: 5′ GUGCGCUGCUGGUGCCAACTT 3′ | 19 |
| | | Antisense: 5′ GUUGGCACCAGCAGCGCACTT 3′ | 20 |
| siNegative control | Target#1 | Sense: 5′ AUCCGCGCGAUAGUACGUA 3′ | 21 |
| (Cocktail) | | Antisense: 5′ UACGUACUAUCGCGCGGAU 3′ | 22 |
| | Target#2 | Sense: 5′ UUACGCGUAGCGUAAUACG 3′ | 23 |
| | | Antisense: 5′ CGUAUUACGCUACGCGUAA 3′ | 24 |
| | Target#3 | Sense: 5′ UAUUCGCGCGUAUAGCGGU 3' | 25 |
| | | Antisense: 5′ ACCGCUAUACGCGCGAAUA 3′ | 26 |
| siWHSC1#1 | | Sense: 5′ CAGAUCUACACAGCGGAUATT 3′ | 27 |
| | | Antisense: 5′ UAUCCGCUGUGUAGAUCUGTT 3′ | 28 |
| | | Target: 5′ CAGATCTACACAGCGGATA 3′ | 29 |
| siWHSC1#2 | | Sense: 5′ GUUAAUUGGCAUAUGGAAUTT 3′ | 30 |
| | | Antisense: 5′ AUUCCAUAUGCCAAUUAACTT 3′ | 31 |
| | | Target: 5′ GTTAATTGGCATATGGAAT 3′ | 32 |
| siWHSC1L1#1 | | Sense: 5′ CUCACAAAUGGGUAUCCAUTT 3′ | 33 |
| | | Antisense: 5′ AUGGAUACCCAUUUGUGAGTT 3′ | 34 |
| | | Target: 5′ CTCACAAATGGGTATCCAT 3′ | 35 |
| siWHSC1L1#2 | | Sense: 5′ GUACUGAAAUUCGGAGAGCATT 3′ | 36 |
| | | Antisense: 5′ UGUCUCCGAAUUUCAGUACTT 3′ | 37 |
| | | Target: 5′GTACTGAAATTCGGAGACA 3′ | 38 |

Flow Cytometry Assays (FACS)

To examine the role of WHSC1 in the cell cycle, SW780 and A549 cells were treated with siWHSC1 s (siWHSC1#1, siWHSC1#2) or control siRNAs (siEGFP and siNC), and cultured in a CO₂ incubator at 37 degrees C. for 72 hours. Aliquots of 1×10⁵ cells were collected by trypsinization, and stained with propidium iodide following the manufacturer's instructions (Cayman Chemical, Ann Arbor, Mich.). Cells were analyzed by FACScan (BECKMAN COULTER, Brea, Calif.) with MultiCycle for Windows software (BECKMAN COULTER) for detailed cell cycle status. The percentages of cells in G₀/G₁, S and G₂/M phases of the cell cycle were determined from at least 20,000 ungated cells.

For more details, a 5′-bromo-2′-deoxyuridine (BrdU) flow kit (BD Pharmingen, San Diego, Calif.) was used to determine the cell cycle kinetics and to measure the incorporation of BrdU into DNA of proliferating cells. The assay was performed according to the manufacturer's protocol. Briefly, cells (2×105 per well) were seeded overnight in 6-well tissue culture plates and treated with an optimized concentration of siRNAs in medium containing 10% FBS for 72 h, followed by addition of 10 micro-M BrdU, and incubations continued for an additional 30 min. Both floating and adherent cells were pooled from triplicate wells per treatment point, fixed in a solution containing paraformaldehyde and the detergent saponin, and incubated for 1 h with DNase at 37 degrees C. (30 micro-g per sample). FITC-conjugated anti-BrdU antibody (1:50 dilution in Wash buffer; BD Pharmingen, San Diego, Calif.) was added and incubation continued for 20 min at room temperature. Cells were washed in Wash buffer and total DNA was stained with 7-amino-actinomycin D (7-AAD; 20 micro-L per sample), followed by flow cytometric analysis using FACScan (BECKMAN COULTER) and total DNA content (7-AAD) was determined CXP Analysis Software Ver. 2.2 (BECKMAN COULTER).

Microarray Hybridization and Statistical Analysis for the Clarification of Down-Stream Genes

Purified total RNA was labeled and hybridized onto Affymetrix GeneChip U133 Plus 2.0 oligonucleotide arrays (Affymetrix, Santa Clara, Calif.) according to the manufacturer's instructions. Probe signal intensities were normalized by RMA and Quantile (using R and Bioconductor). Next, signal intensity fluctuation due to inter-experimental variation was estimated. Each experiment was replicated (1 and 2), and the standard deviation (stdev) of log₂(intensity₂/intensity₁) was calculated for each of a set of intensity ranges with the midpoints being at log₂((intensity₁+intensity₂)/2)=5, 7, 9, 11, 13, and 15. The present inventors modeled intensity variation using the formula stdev(log₂(intensity₂/intensity₁))=a*(log₂((intensity₁+intensity₂)/2))+b and estimated parameters a and b using the method of least squares. Using these values, the standard deviation of intensity fluctuation was calculated. The signal intensities of each probe were then compared between siWHSC1 (EXP) and controls (EGFP/FFLuc) (CONT) and tested for up/down-regulation by calculating the z-score: log₂(intensity_{EXP}/intensity_{CONT})/(a*(log₂((intensity_{EXP}+intensity_{CONT})/2))+b). Resultant P values for the replication sets were multiplied to calculate the final P value of each probe. These procedures were applied to each comparison: siEGFP vs. siWHSC1, siFFLuc vs. siWHSC1, and siEGFP vs. siFFLuc, respectively. The present inventors determined up and down-regulated gene sets as those that simultaneously satisfied the following criteria: (1) The Benjamini-Hochberg false discovery rate (FDR)<=0.05 for EGFP vs. siWHSC1, (2) FDR<=0.05 for FFLuc vs. siWHSC1 and the regulation direction is the same as (1), and (3) EGFP vs. FFLuc has the direction opposite to (1) and (2) or P>0.05 for EGFP vs. FFLuc. Finally, the present inventors performed a pathway analysis using the hyper-geometric distribution test, which calculates the probability of overlap between the up/down-regulated gene set and each GO category compared against another gene list that is randomly sampled. The present inventors applied the test to the identified up/down-regulated genes to test whether or not they are significantly enriched (FDR<=0.05) in each category of “Biological processes” (857 categories) as defined by the Gene Ontology database.

Chromatin Immunoprecipitation Assay (ChIP)

ChIP assays were performed using ChIP Assay kit (Millipore, Billerica, Mass.) according to the manufacture's protocol. Briefly, the fragment of WHSC1 and chromatin complexes was immunoprecipitated with anti-FLAG antibody 48 h after transfection with pCAGGS-n3FC (mock), pCAGGS-n3FC-WHSC1 wt (WHSC1 wt) and pCAGGS-n3FC-WHSC1[delta]SET (WHSC1[delta]SET) vectors. After the bound DNA fragments to WHSC1 wt or WHSC1[delta]SET were eluted, and the amount was subjected to quantitative real-time PCR reactions. Primer sequences are shown in Table 3.

TABLE 3
Primer sequences for ChIP assay

| | | SEQ ID |
|---|---|---|
| Primer name | Primer sequence | NO. |
| Ch1-forward | 5′ CAGTAACGTCACACGGACTAC 3′ | 53 |
| Ch1-reverse | 5′ CGCTCCCTCGCGCTCTTCTGC 3′ | 54 |
| Ch2-forward | 5′ CCCCTCTTCCCTGGCGGGGAG 3′ | 55 |
| Ch2-reverse | 5′ GCCCAAAAGCCATCCCTGAGG 3′ | 56 |
| Ch3-forward | 5′ GTGGTCTCCCCAGGCTGCGTG 3′ | 57 |
| Ch3-reverse | 5′ AGGGGTGCAGGGGGCCCCGTC 3′ | 58 |
| Ch4-forward | 5′ GCAGTCGCTGAGATTCTTTGG 3′ | 59 |
| Ch4-reverse | 5′ ACCACGAGAAGGGGTGACTGG 3′ | 60 |
| Ch5-forward | 5′ CGCCCCTGTGCGCCCGGAATG 3′ | 61 |
| Ch5-reverse | 5′ TCAGCGACTGCATCTTCTTTC 3′ | 62 |

Luciferase Assays for TOPFLASH and FOPFLASH Reporter Activities

The luciferase assays were performed using Dual-Luciferase Reporter Assay System (Promega) according to the manufacturer's protocol. 293T cells were cultured on 24-well microplates and co-transfected with mock, pCAGGS-WHSC1, TOPFLASH, FOPFLASH and pRL-TK, which was used as an internal control, vectors in a suitable combination. Cells were lysed 24 h after transfection for analysis, and luciferase activity was measured with a luminometer (BERTHOLD TECHNOLOGIES, Bad Wildbad, Germany). Further, 293T cells were cultured on 24-well microplates and cotransfected with pCAGGS-n3FC (mock), pCAGGS-n3FC-WHSC1 wt (WHSC1 wt), pCAGGS-n3FC-WHSC1[delta]SET (WHSC1[delta]SET) TOPFLASH, FOPFLASH and pRL-TK, which was used as an internal control, vectors in a suitable combination. Cells were lysed 48 h after transfection for analysis, and luciferase activity was measured with a luminometer (BERTHOLD TECHNOLOGIES, Bad Wildbad, Germany).

### Example 2

### Overexpression of WHSC1 in Clinical Cancer Tissues

Through examinations of the level of histone lysine methyltransferase genes in a small subset of British clinical bladder cancer samples, the present inventors found significant overexpression of WHSC1 as well as WHSC1L1 in the cancer samples compared with non-cancerous samples. Subsequently, the present inventors analyzed 120 bladder cancer samples and 22 normal control samples (British), and confirmed significant elevation of WHSC1 expression levels in tumor cells compared with normal cells (both P=0.0002, Mann-Whitney U test, FIG. 1A). WHSC1 expression appeared to be high at the advanced stage (pT3 and pT4), but subclassification of tumors according to tumor grade, metastasis status, gender, recurrence status and smoking history identified but no significant difference at other factors (Table 4). Further, significant elevation of WHSC1 and WHSC1L1 expression levels in tumor cells compared with normal cells was confirmed (both P<0.0001, Mann-Whitney U test). Subclassification of tumors according to tumor grade, metastasis status, gender, recurrence status and smoking history identified no significant difference in their expression levels (Table 5). To evaluate protein expression levels of WHSC1 and WHSC1L1 in bladder tissues, the present inventors performed immunohistochemical analysis using anti-WHSC1 and anti-WHSC1L antibody, and observed their strong staining in the nucleus of malignant cells, but weak or absent staining in non-neoplastic tissues (FIG. 1B). In addition, previous microarray expression analysis of a large number of clinical samples (Kikuchi T et al. Oncogene 22: 2192-2205, 2003, Nakamura T et al. Oncogene 23: 2385-2400, 2004, Nishidate T et al. Int J Oncol 25: 797-819, 2004., Takata Ret al. Clin Cancer Res 11: 2625-2636, 2005) indicated that WHSC1 expression was significantly up-regulated in various types of cancer, including bladder cancer, breast cancer, prostate cancer, renal cancer, small cell lung cancer (SCLC) and pancreas cancer and that elevated WHSC1L1 expression was also observed in breast cancer, CML, lymphoma and lung cancer (FIG. 1C, Table 6).

TABLE 4
Statistical analysis of WHSC1 expression levels in clinical bladder tissues

| | | WHSC1 | | |
|---|---|---|---|---|
| Characteristic | Case (n) | Mean | SD | 95% CI |
| Normal (Control) | 22 | 0.234 | 0.097 | 0.193-0.274 |
| Tumor (Total) | 120 | 0.666 | 1.061 | 0.476-0.856 |
| Tumor stage | | | | |
| pTa, pT1 | 85 | 0.683 | 1.122 | 0.445-0.922 |
| pT2 | 26 | 0.516 | 0.411 | 0.358-0.674 |
| pT3, pT4 | 6 | 1.175 | 2.085 | −0.494-2.843 |
| Tumor grade | | | | |
| G1 | 12 | 0.794 | 1.227 | 0.100-1.488 |
| G2 | 60 | 0.643 | 1.168 | 0.347-0.938 |
| G3 | 47 | 0.666 | 0.894 | 0.411-0.922 |
| Metastasis | | | | |
| Negative | 93 | 0.684 | 1.150 | 0.450-0.918 |
| Positive | 27 | 0.604 | 0.685 | 0.345-0.862 |
| Gender | | | | |
| Male | 88 | 0.643 | 0.824 | 0.471-0.816 |
| Female | 30 | 0.680 | 1.594 | 0.110-1.251 |
| Recurrence | | | | |
| No | 27 | 0.594 | 1.028 | 0.206-0.981 |
| Yes | 49 | 0.601 | 0.818 | 0.372-0.830 |
| Died | 8 | 1.543 | 2.974 | −0.518-3.604 |
| Smoke | | | | |
| No | 27 | 0.782 | 1.061 | 0.382-1.183 |
| Yes | 48 | 0.725 | 1.434 | 0.319-1.131 |

TABLE 5
Statistical analysis of WHSC1 and WHSC1L1 expression levels in clinical bladder tissues

| | | WHSC1 | | | | WHSC1L1 | | |
|---|---|---|---|---|---|---|---|---|
| Characteristic | Case (n) | Mean | SD | 95% CI | Case (n) | Mean | SD | 95% CI |
| Normal (Control) | 22 | 0.219 | 0.086 | 0.183-0.254 | 22 | 1.593 | 0.397 | 1.427-1.759 |
| Tumor (Total) | 120 | 0.527 | 0.681 | 0.406-0.649 | 120 | 5.537 | 8.376 | 4.039-7.036 |
| Tumor stage | | | | | | | | |
| pTa, pT1 | 85 | 0.538 | 0.725 | 0.384-0.692 | 85 | 6.181 | 9.670 | 4.125-8.236 |
| pT2 | 25 | 0.424 | 0.297 | 0.308-0.541 | 25 | 4.527 | 3.658 | 3.093-5.961 |
| pT3, pT4 | 6 | 0.839 | 1.255 | −0.165-1.843 | 6 | 3.169 | 1.814 | 1.717-4.620 |
| Tumor grade | | | | | | | | |
| G1 | 12 | 0.684 | 1.005 | 0.115-1.253 | 12 | 6.987 | 6.892 | 3.088-10.887 |
| G2 | 60 | 0.495 | 0.715 | 0.314-0.676 | 60 | 5.782 | 8.458 | 3.642-7.922 |
| G3 | 47 | 0.530 | 0.541 | 0.376-0.685 | 47 | 4.857 | 8.792 | 2.343-7.370 |
| Metastasis | | | | | | | | |
| Negative | 93 | 0.544 | 0.744 | 0.393-0.695 | 93 | 5.433 | 7.412 | 3.926-6.939 |
| Positive | 27 | 0.469 | 0.400 | 0.318-0.620 | 27 | 5.898 | 11.251 | 1.654-10.142 |
| Gender | | | | | | | | |
| Male | 88 | 0.505 | 0.562 | 0.388-0.623 | 88 | 6.155 | 9.453 | 4.197-8.130 |
| Female | 30 | 0.527 | 0.941 | 0.190-0.863 | 30 | 3.727 | 3.908 | 2.329-5.126 |
| Recurrence | | | | | | | | |
| No | 27 | 0.431 | 0.627 | 0.194-0.667 | 27 | 7.635 | 11.681 | 3.229-12.041 |
| Yes | 49 | 0.496 | 0.615 | 0.324-0.668 | 49 | 5.574 | 8.832 | 3.101-8.046 |
| Died | 8 | 1.014 | 1.691 | −0.158-2.186 | 8 | 8.263 | 8.403 | 2.441-14.086 |
| Smoke | | | | | | | | |
| No | 27 | 0.620 | 0.708 | 0.353-0.887 | 27 | 6.446 | 6.318 | 4.063-8.828 |
| Yes | 48 | 0.542 | 0.883 | 0.292-0.792 | 48 | 6.509 | 12.013 | 3.110-9.907 |

TABLE 6
Expression of WHSC1 and WHSC1L1 in cancer tissues analyzed by cDNA microarray*

| | | | Ratio (Tumor/Normal) | | | |
|---|---|---|---|---|---|---|
| | | | Count >2 | Count >3 | Count >5 | Count >10 |
| | | Case (n) | (T/N) | (T/N) | (T/N) | (T/N) |
| WHSC1 | | | | | | |
| Tissue type | | | | | | |
| Bladder cancer | | 32 | 26 (81.3%) | 18 (56.3%) | 15 (46.9%) | 3 (9.4%) |
| Breast cancer | | 40 | 31 (77.5%) | 22 (55%) | 8 (20%) | 1 (2.5%) |
| Cholangio cellular | | 15 | 6 (40%) | 4 (26.7%) | 1 (6.7%) | 1 (6.7%) |
| carcinoma | | | | | | |
| CML | | 56 | 37 (66.1%) | 27 (48.2%) | 16 (28.6%) | 10 (17.9%) |
| Esophageal cancer | | 18 | 9 (50%) | 5 (27.8%) | 3 (16.7%) | 0 (0%) |
| HCC | | 14 | 8 (57.1%) | 5 (35.7%) | 5 (35.7%) | 2 (14.3%) |
| Lung cancer | NSCLC | 28 | 10 (35.7%) | 5 (17.9%) | 2 (7.1%) | 2 (7.1%) |
| | SCLC | 15 | 14 (93.3%) | 12 (80%) | 8 (53.3%) | 1 (6.7%) |

| | | | | | |
|---|---|---|---|---|---|
| Osteosarcoma | 16 | 9 (56.3%) | 7 (35.7%) | 4 (25%) | 3 (18.8%) |
| Pancreatic cancer | 13 | 12 (92.3%) | 11 (84.6%) | 10 (76.9%) | 7 (53.8%) |
| Prostate cancer | 38 | 18 (47.4%) | 9 (23.7%) | 5 (13.2%) | 1 (2.6%) |
| Renal cell carcinoma | 19 | 8 (42.1%) | 5 (26.3%) | 3 (13.2%) | 0 (0%) |
| Soft tissue tumor | 52 | 19 (36.5%) | 10 (19.2%) | 4 (7.7%) | 2 (3.8%) |
| WHSC1L1 | | | | | |
| Tissue type | | | | | |
| Breast cancer | 3 | 3 (100%) | 3 (100%) | 2 (66.7%) | 0 (0%) |
| CML | 24 | 18 (75%) | 15 (62.5%) | 14 (58.4%) | 11 (45.8%) |
| Lymphoma | 3 | 3 (100%) | 3 (100%) | 3 (100%) | 2 (66.7%) |
| Small cell lung cancer | 4 | 4 (100%) | 3 (75%) | 2 (50%) | 1 (25%) |

*The signal intensity of WHSC1 and WHSC1L1 between tumor tissues and corresponding nonneoplatic tissues derived from the same patient were compared.

### Example 3

### WHSC1 Protein was Significantly Upregulated in a Number of Bladder and Lung Tumor Tissues

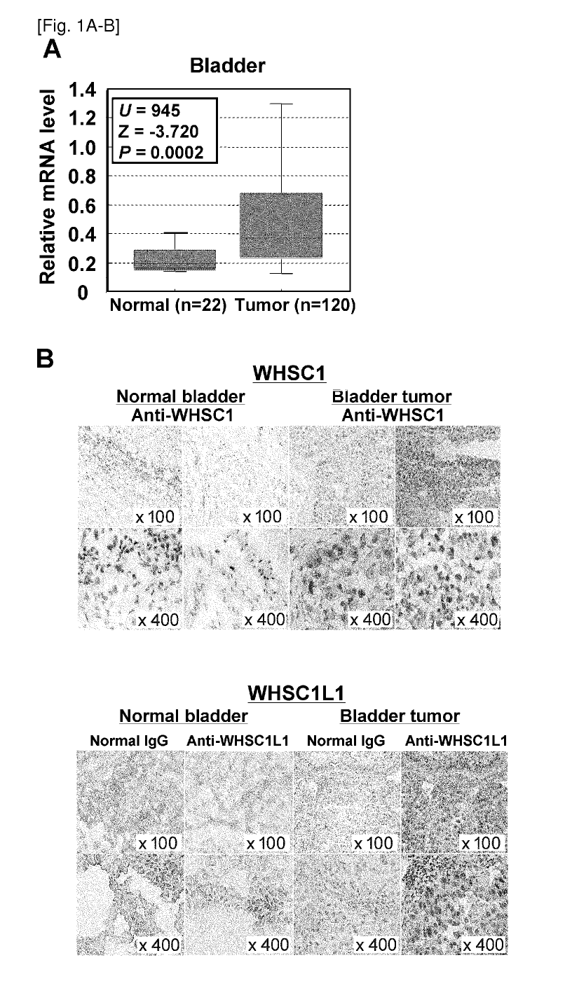

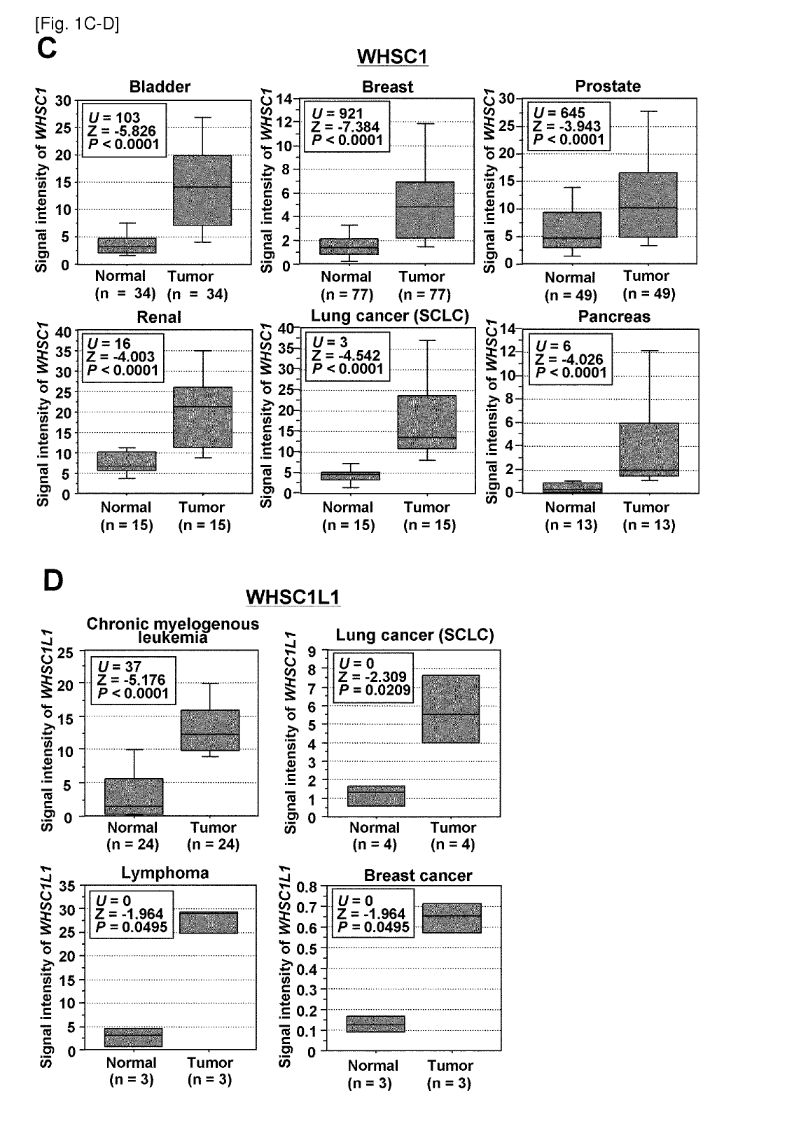

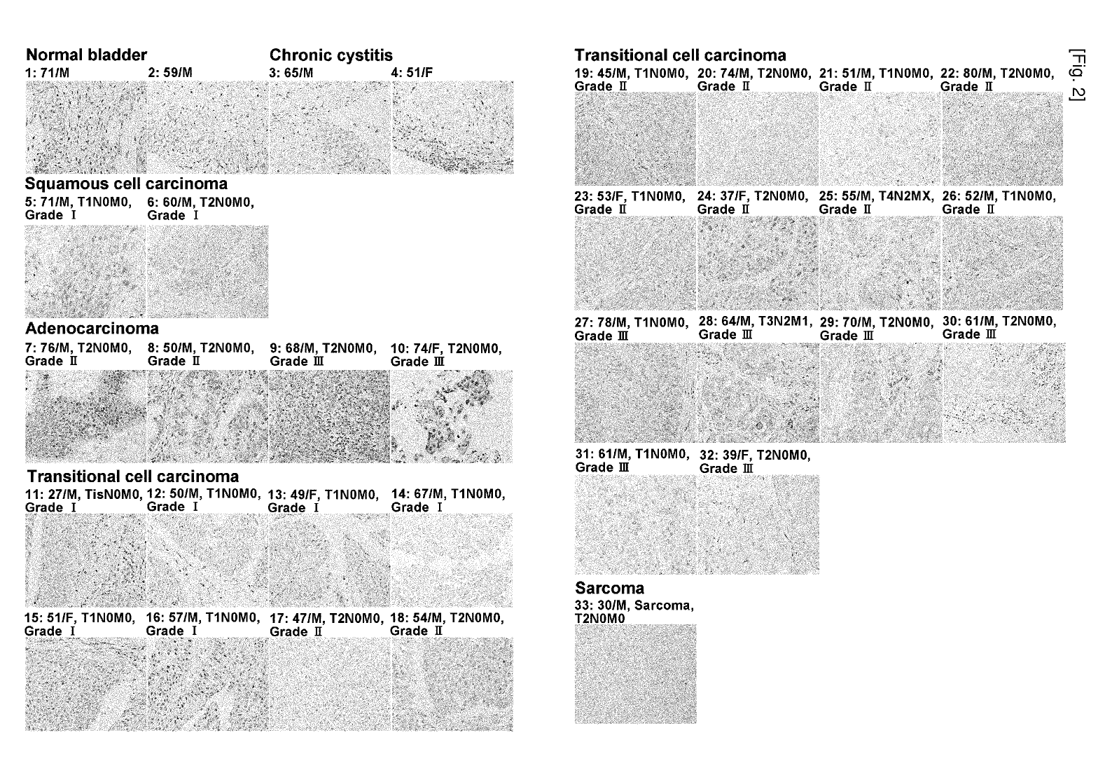

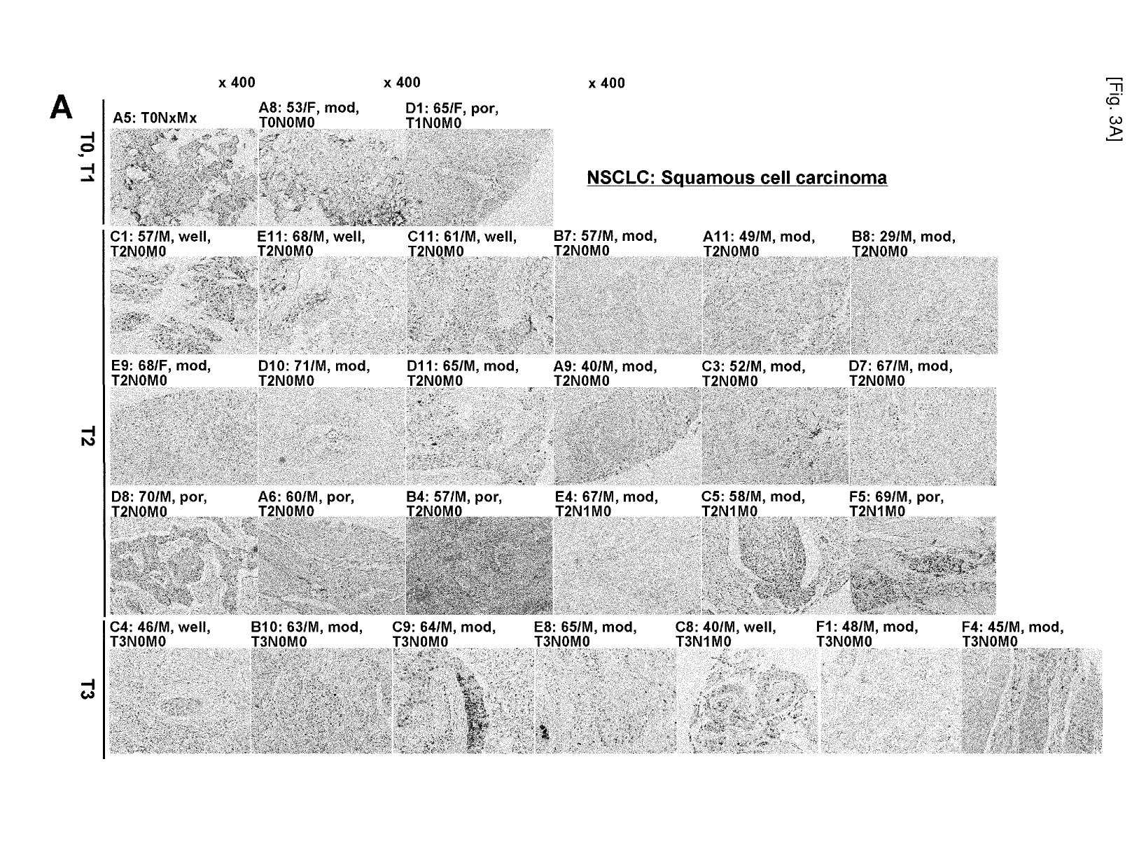

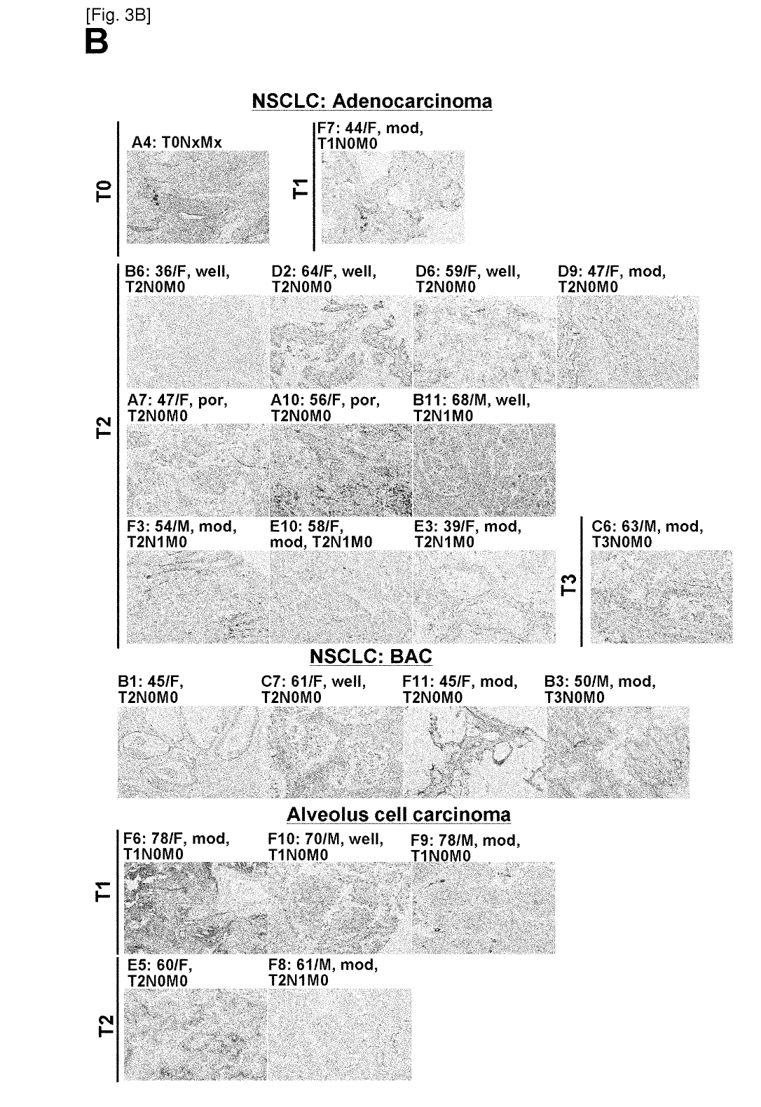

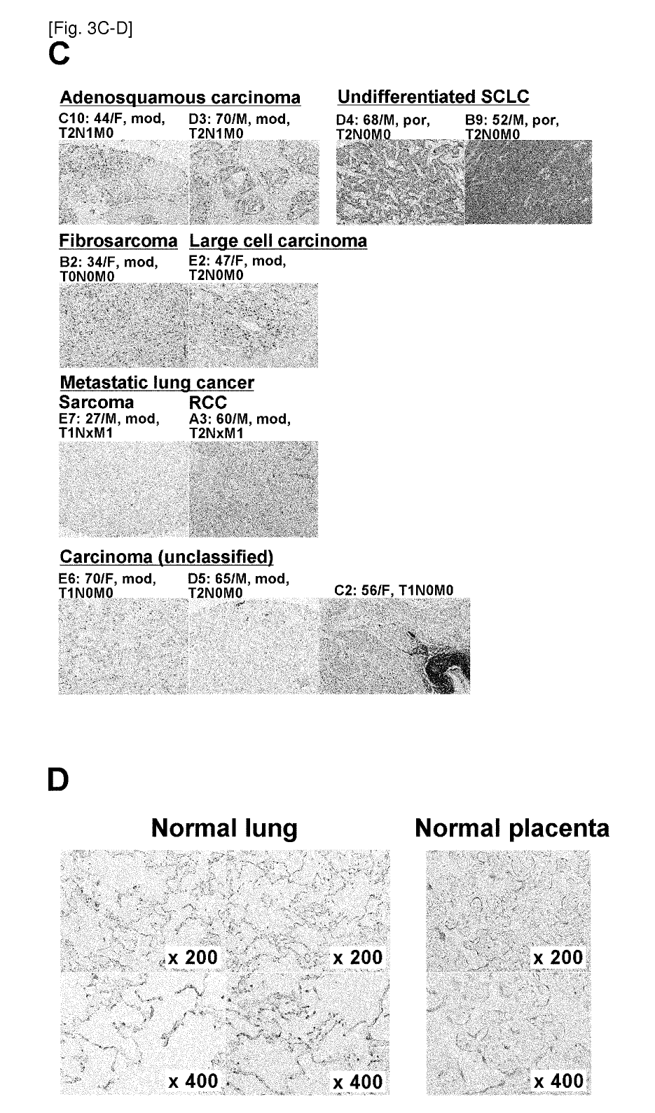

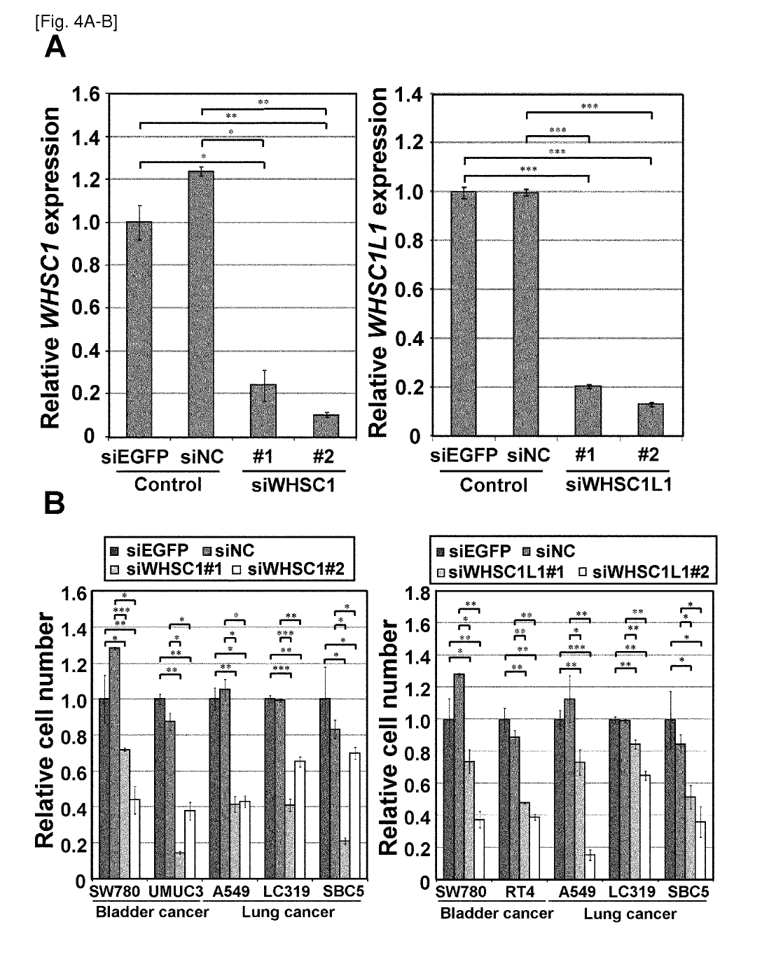

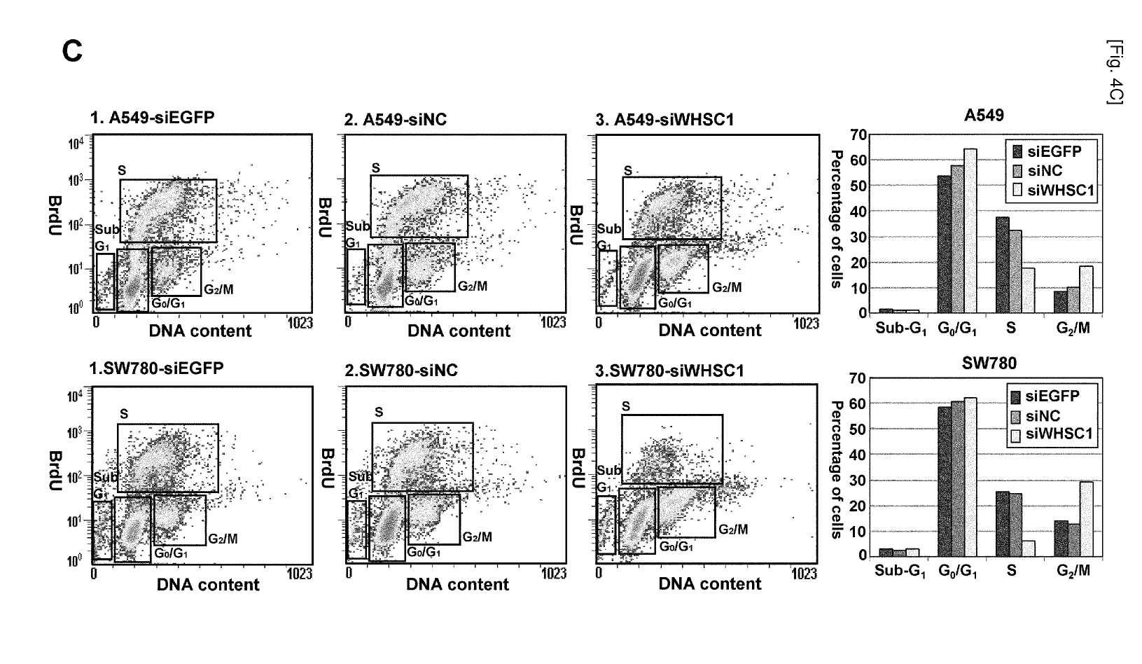

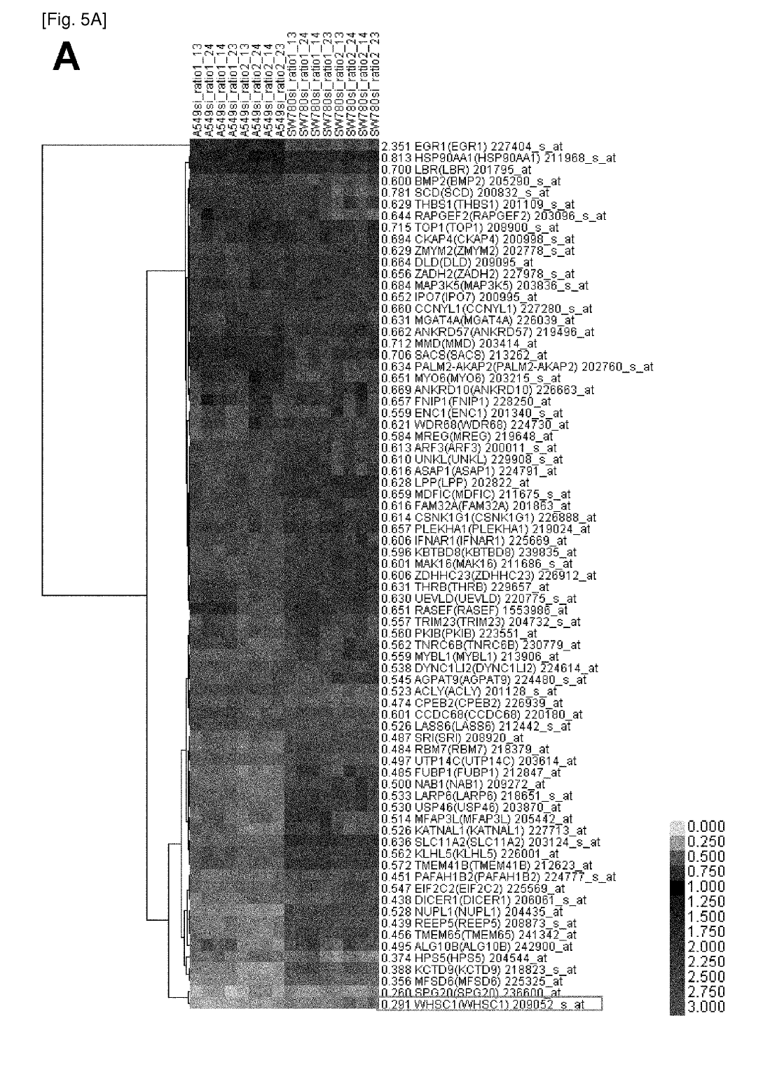

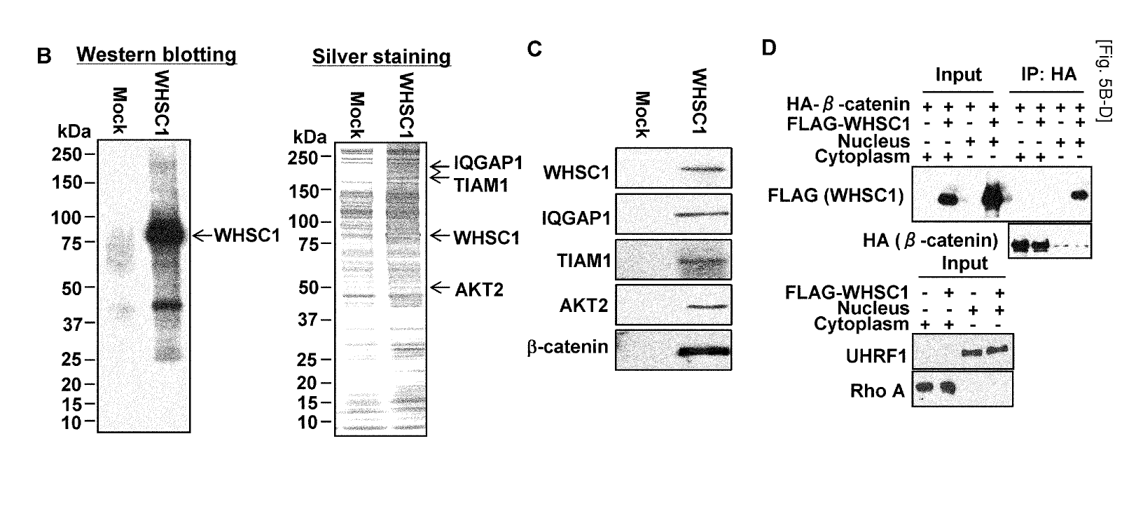

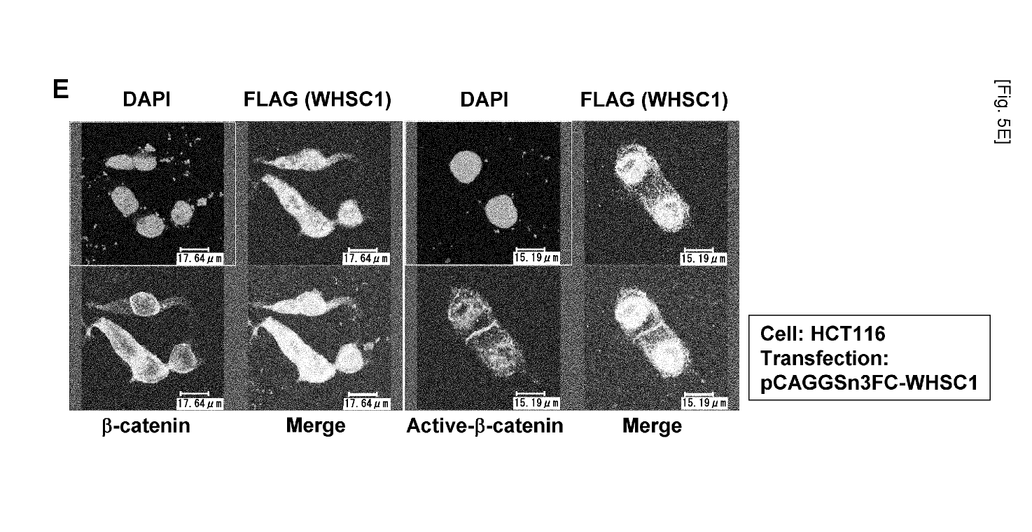

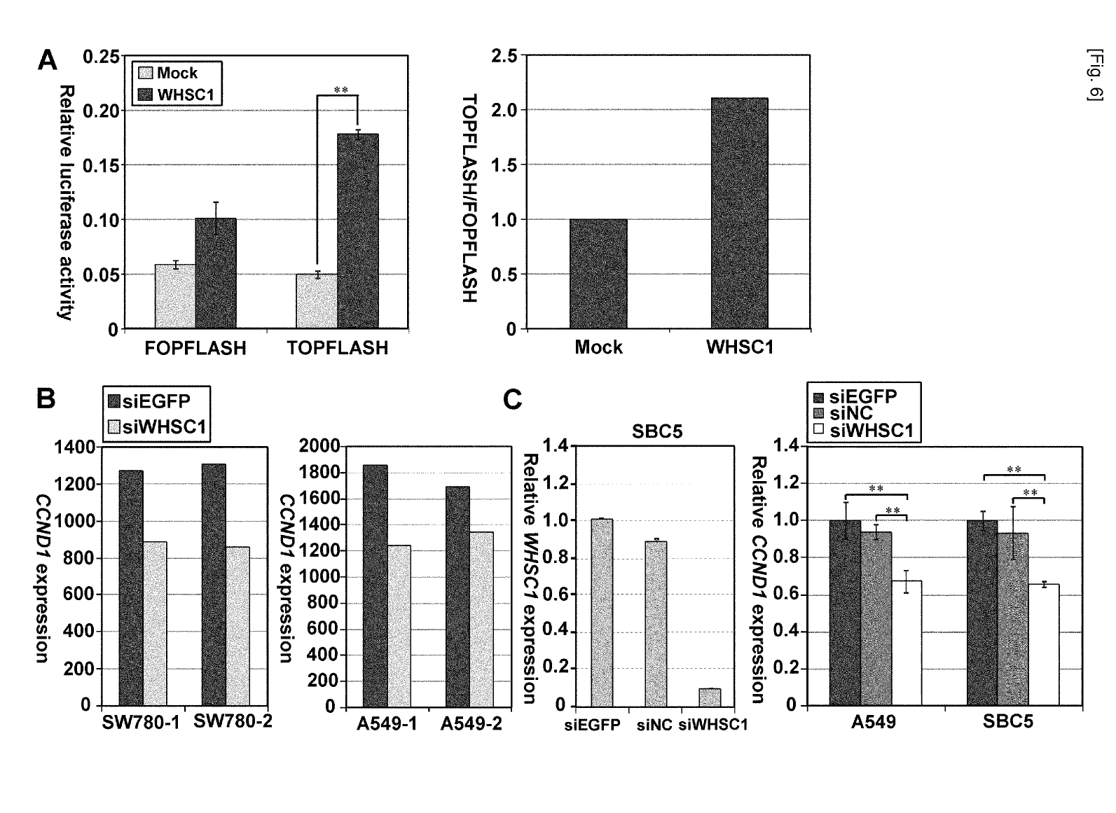

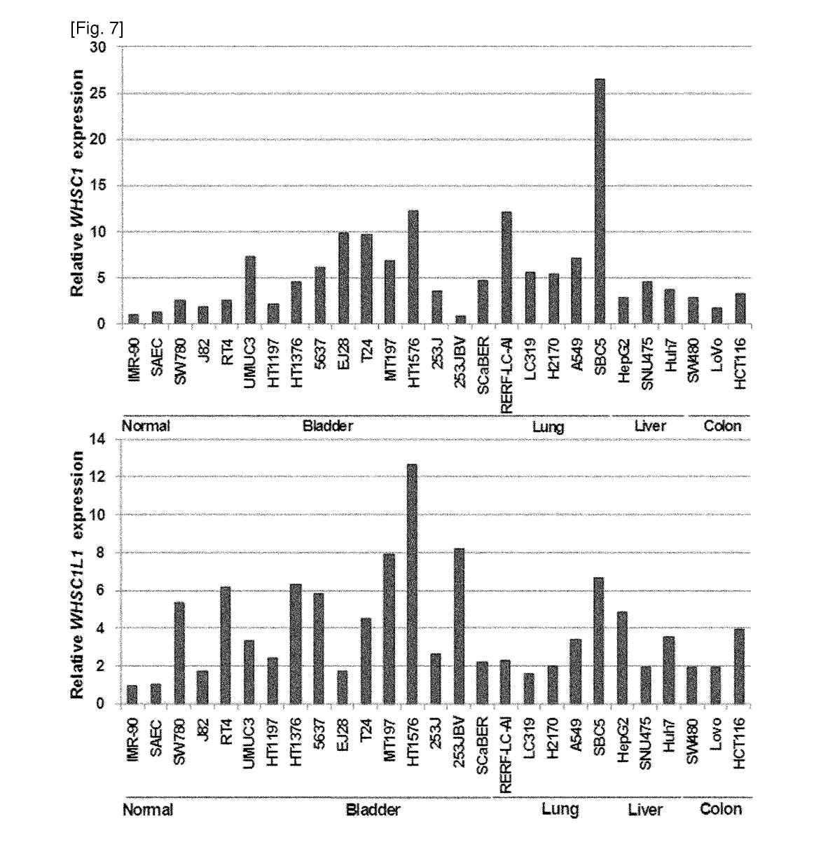

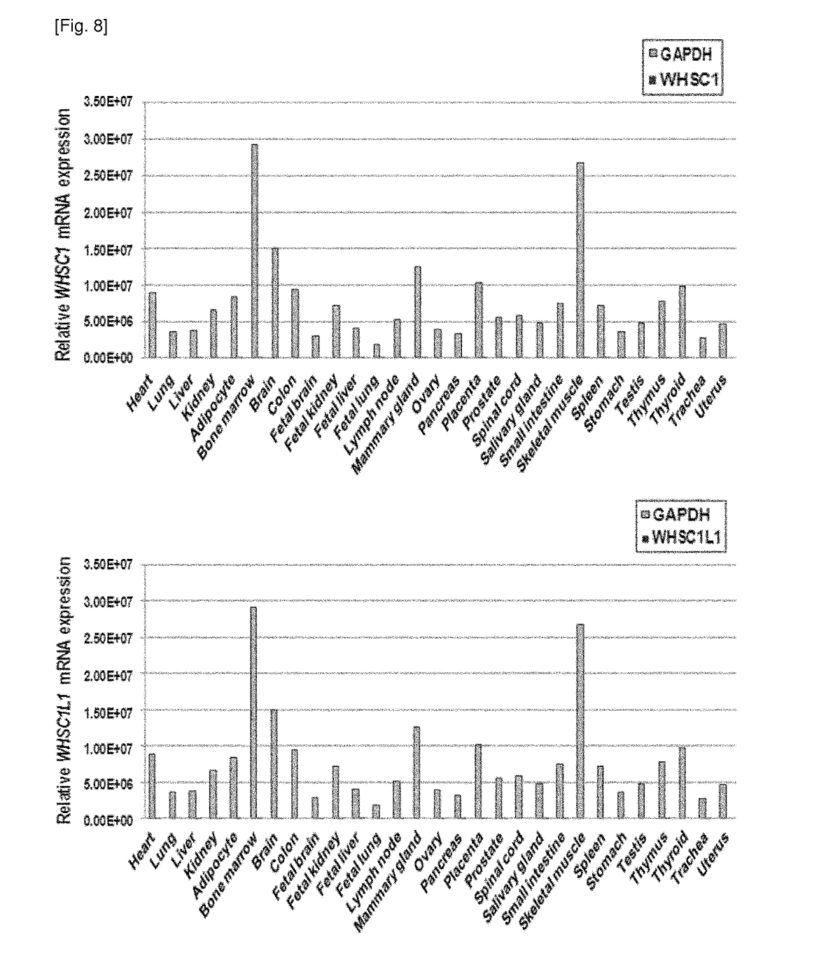

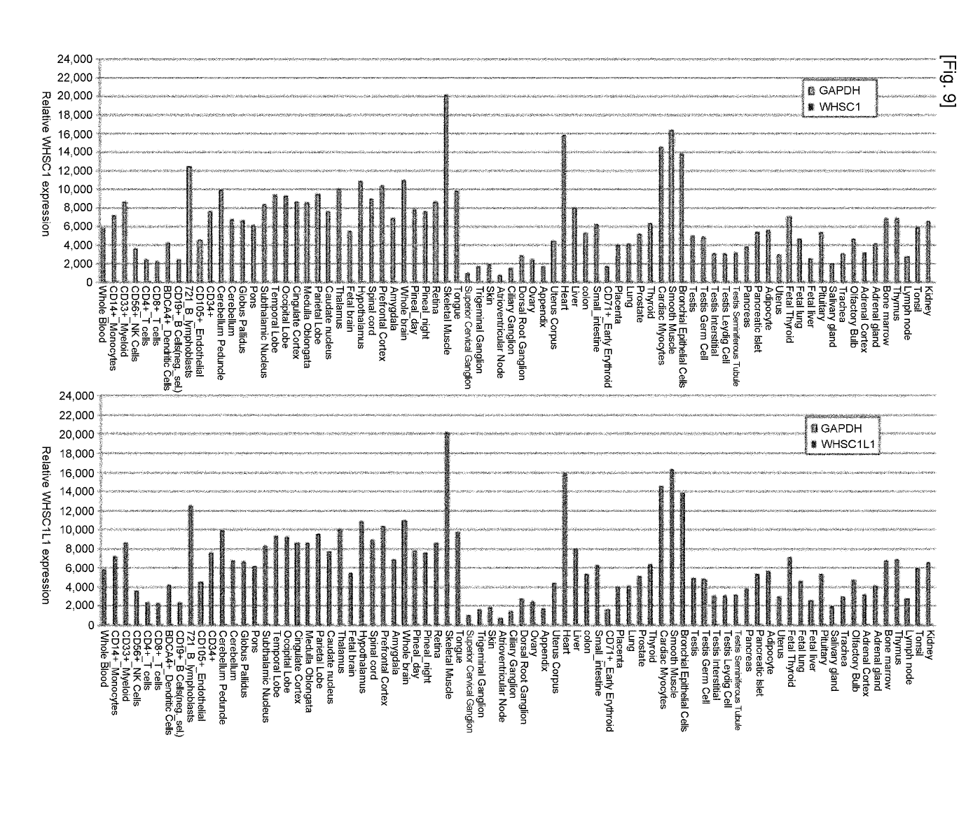

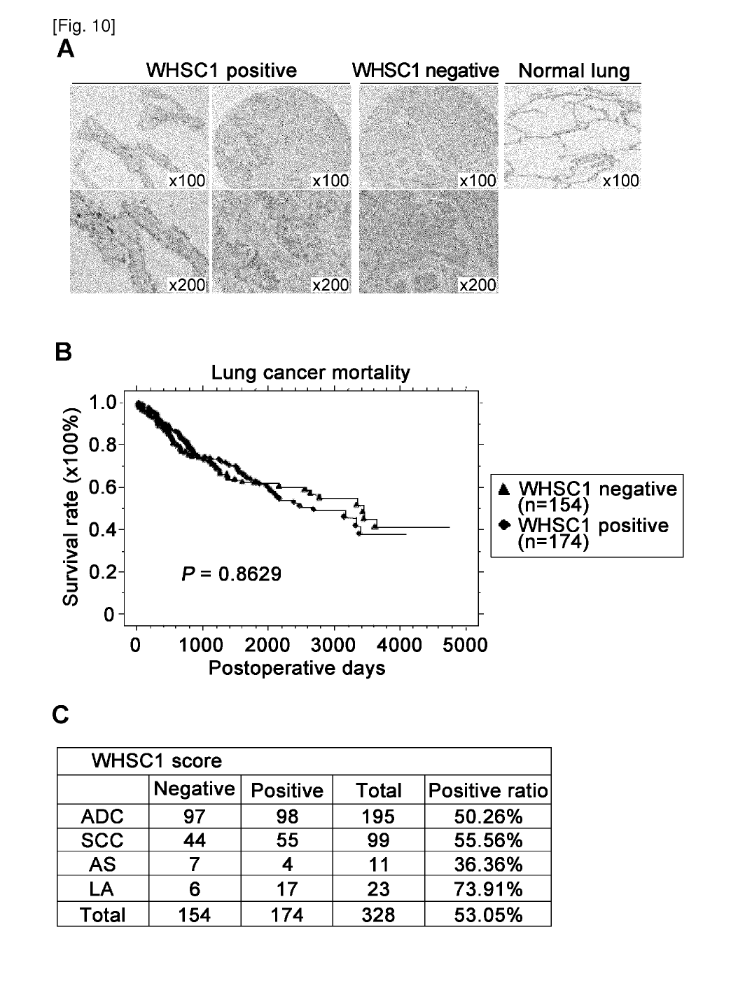

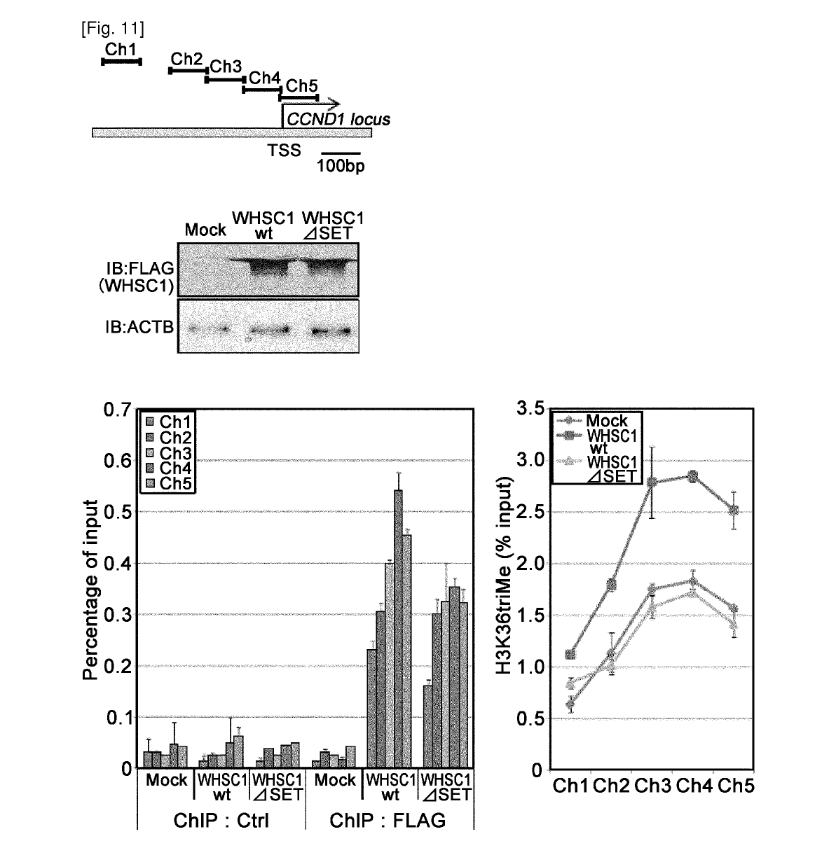

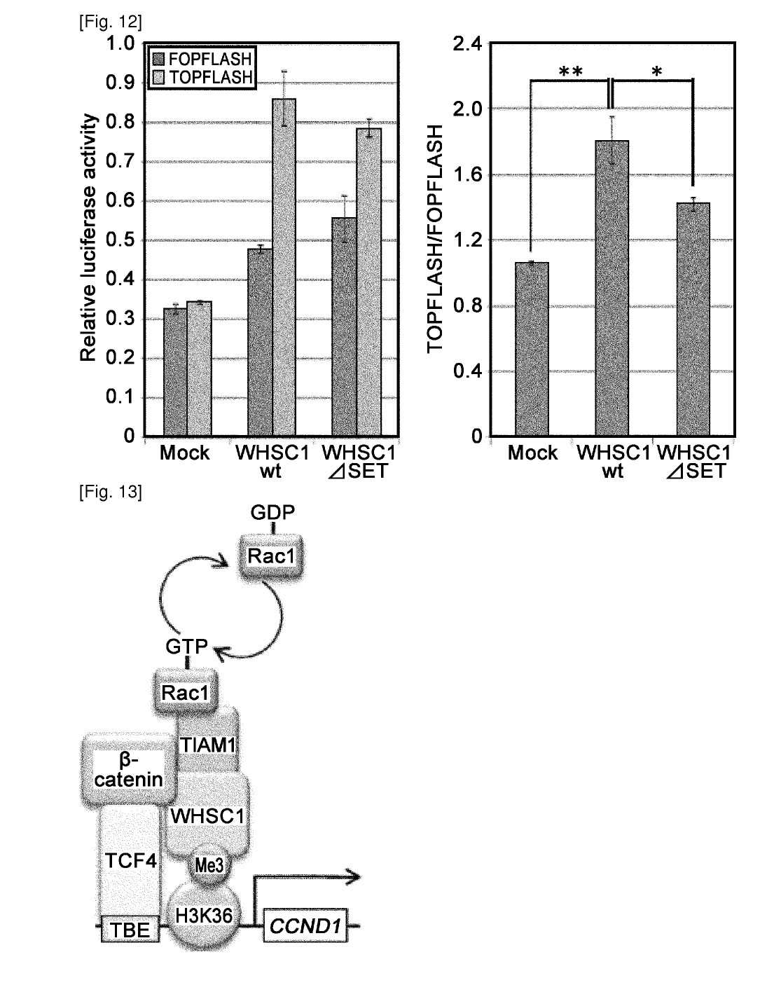

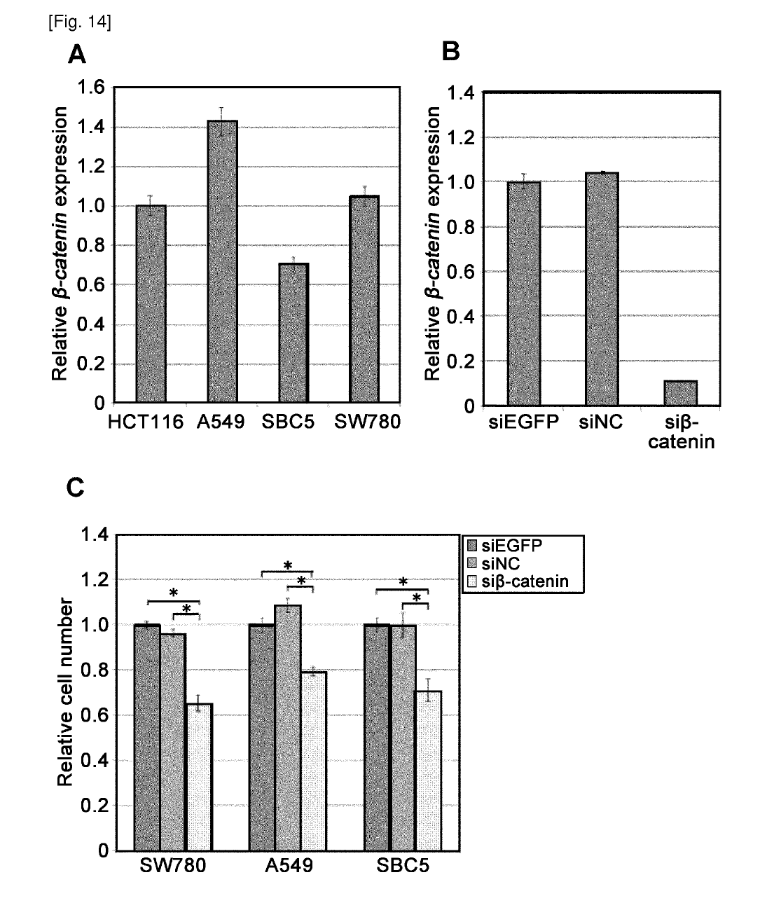

In order to further validate protein expression levels of WHSC1 in bladder tissues, tissue microarray experiments using 29 bladder tissue sections was conducted (FIG. 2, Table 7), and detected its strong staining in 17 cases, and weak or moderate staining was observed in 9 cases. Moreover, we found no significant relationship between WHSC1 protein expression levels and clinicopathologic characteristics, consistent with our real-time PCR results. In addition, we measured expression levels of WHSC1 in various histological types of lung tumor tissues by tissue microarray (FIG. 3, Table 8). Among 62 tumor tissue sections examined, we observed strong staining in 19 cases, and weak or moderate staining in 24 cases. To analyze the association of WHSC1 expression with clinical outcomes in more detail, we further performed tumor tissue microarray containing 328 archival non-small-cell lung cancers (NSCLC) (FIG. 10). WHSC1 stained positively in 174 cases (53.0%) and negatively in 154 cases (47.0%). Meanwhile, no significant statistical significance was observed between WHSC1-positivity and any patients' characteristics (Table 9). Then, univaridate analysis was applied to evaluate association between prognosis and WHSC1 expression, but no statistical significance was observed (P=0.8629 by log-rank test; FIG. 10B and Table 10). These results reveal that WHSC1 is frequently overexpressed in lung cancer regardless of clinical characteristics, and it doesn't serve as a prognostic marker.

TABLE 7
Clinicopathological characterics of bladder tissues on the tissue microarry

| | | | | | Stage | WHSC1 |
|---|---|---|---|---|---|---|
| Case No. | Age | Gender | Histology | Grade | (TNM) | expression |
| 1 | 71 | M | Normal | — | — | − |
| 2 | 59 | M | Normal | — | — | − |
| 3 | 65 | M | Chronic cystitis | — | — | − |
| 4 | 51 | F | Chronic cystitis | — | — | − |
| 5 | 71 | M | Squamous cell carcinoma | I | T1N0M0 | ++ |
| 6 | 60 | M | Squamous cell carcinoma | I | T2N0M0 | ++ |
| 7 | 76 | M | Adenocarcinoma | II | T2N0M0 | ++ |
| 8 | 50 | M | Adenocarcinoma | II | T2N0M0 | ++ |
| 9 | 68 | M | Adenocarcinoma | III | T2N0M0 | ++ |
| 10 | 74 | F | Adenocarcinoma | III | T2N0M0 | ++ |
| 11 | 27 | M | Transitional cell carcinoma | I | TisN0M0 | + |
| 12 | 50 | M | Transitional cell carcinoma | I | T1N0M0 | − |
| 13 | 49 | F | Transitional cell carcinoma | I | T1N0M0 | + |
| 14 | 67 | M | Transitional cell carcinoma | I | T1N0M0 | + |
| 15 | 51 | F | Transitional cell carcinoma | I | T1N0M0 | ++ |
| 16 | 57 | M | Transitional cell carcinoma | I | T1N0M0 | ++ |
| 17 | 47 | M | Transitional cell carcinoma | II | T2N0M0 | ++ |
| 18 | 54 | M | Transitional cell carcinoma | II | T2N0M0 | ++ |
| 19 | 45 | M | Transitional cell carcinoma | II | T1N0M0 | ++ |
| 20 | 74 | M | Transitional cell carcinoma | II | T2N0M0 | − |
| 21 | 51 | M | Transitional cell carcinoma | II | T1N0M0 | + |
| 22 | 80 | M | Transitional cell carcinoma | II | T2N0M0 | + |
| 23 | 53 | F | Transitional cell carcinoma | II | T1N0M0 | − |
| 24 | 37 | M | Transitional cell carcinoma | II | T2N0M0 | ++ |
| 25 | 55 | M | Transitional cell carcinoma | II | T4N2MX | + |
| 26 | 52 | M | Transitional cell carcinoma | II | T1N0M0 | + |
| 27 | 78 | M | Transitional cell carcinoma | III | T1N0M0 | ++ |
| 28 | 64 | M | Transitional cell carcinoma | III | T3N2M1 | ++ |
| 29 | 70 | M | Transitional cell carcinoma | III | T2N0M0 | ++ |
| 30 | 61 | M | Transitional cell carcinoma | III | T2N0M0 | + |
| 31 | 61 | M | Transitional cell carcinoma | III | T1N0M0 | ++ |
| 32 | 39 | F | Transitional cell carcinoma | III | T2N0M0 | + |
| 33 | 30 | M | Sarcoma | — | T2N0M0 | ++ |

(−) negative expression
(+) low or moderate expression
(++) high expression

TABLE 8
Clinicopathological characterics of lung tissues on the tissue microarry

| | | | | | Stage | WHSC1 |
|---|---|---|---|---|---|---|
| Case No. | Age | Gender | Histology | Differentiation | (TNM) | expression |
| A1 | 29 | F | Human Normal Placenta | | | − |
| A2 | | | | | | − |
| A3 | 60 | M | Pulmonary metastases renal cell carcinoma | Moderately | T2NxM1 | + |
| A4 | N/A | N/A | Adenocarcinoma | | T0NxMx | + |
| A5 | N/A | N/A | Squamous cell carcinoma | | T0NxMx | − |
| A6 | 60 | M | Squamous cell carcinoma | Poorly | T2N0M0 | − |
| A7 | 47 | F | Adenocarcinoma | Poorly | T2N0M0 | − |
| A8 | 53 | F | Squamous cell carcinoma | Moderately | T0N0M0 | + |
| A9 | 40 | M | Squamous cell carcinoma | Moderately | T2N0M0 | + |
| A10 | 56 | F | Adenocarcinoma | Poorly | T2N0M0 | ++ |
| A11 | 49 | M | Squamous cell carcinoma | Moderately | T2N0M0 | − |
| B1 | 45 | F | Bronchio alveolar carcinoma | N/A | T2N0M0 | − |
| B2 | 34 | F | Fibrosarcoma | Moderately | T0N0M0 | ++ |
| B3 | 50 | M | Bronchio alveolar carcinoma | N/A | T3N0M0 | + |
| B4 | 57 | M | Squamous cell carcinoma | Poorly | T2N0M0 | ++ |
| B5 | 65 | M | Atypical Carcinoma, (central type) | Moderately | T3N0M0 | + |
| B6 | 36 | F | Adenocarcinoma, mucous | Well | T2N0M0 | − |
| B7 | 57 | M | Squamous cell carcinoma | Moderately | T2N0M0 | − |
| B8 | 29 | M | Squamous cell carcinoma | Moderately | T2N0M0 | + |
| B9 | 52 | M | Undifferentiated small cell carcinoma | Poorly | T2N0M0 | ++ |
| B10 | 63 | M | Squamous cell carcinoma,(cornifying) | Moderately | T3N0M0 | + |
| B11 | 68 | M | Adenocarcinoma, papillary (peripheral type) | Well | T2N1M0 | ++ |
| C1 | 57 | M | Squamous cell carcinoma, (center type) | Well | T2N0M0 | ++ |
| C2 | 56 | F | Tuberculosis | N/A | T1N0M0 | − |
| C3 | 52 | M | Squamous cell carcinoma | Moderately | T2N0M0 | + |
| C4 | 46 | M | Squamous cell carcinoma, (comifying) | Well | T3N0M0 | + |
| C5 | 58 | M | Squamous cell carcinoma, (central type) | Moderately | T2N1M0 | ++ |
| C6 | 63 | M | Adenocarcinoma | Moderately | T3N0M0 | + |
| C7 | 61 | F | Bronchio alveolar carcinoma | Well | T2N0M0 | + |
| C8 | 40 | M | Squamous cell carcinoma | Well | T3N1M0 | ++ |
| C9 | 64 | M | Squamous cell carcinoma | Moderately | T3N0M0 | ++ |
| C10 | 44 | F | Adenosqumous carcinoam | Moderately | T2N1M0 | + |
| C11 | 61 | M | Squamous cell carcinoma | Well | T2N0M0 | − |
| D1 | 65 | F | Squamous cell carcinoma | Poorly | T1N0M0 | ++ |
| D2 | 64 | F | Adenocarcinoma, papillary (peripheral type) | Well | T2N0M0 | − |
| D3 | 70 | M | Adenosquamous carcinoma | Moderately | T2N1M0 | ++ |
| D4 | 68 | M | Undifferentiated small cell carcinoma | Poorly | T2N0M0 | − |
| D5 | 65 | M | Carcinoma, (peripheral type) | Moderately | T2N0M0 | + |
| D6 | 59 | F | Adenocarcinoma, papillary | Well | T2N0M0 | + |
| D7 | 67 | M | Squamous cell carcinoma | Moderately | T2N0M0 | ++ |
| D8 | 70 | M | Squamous cell carcinoma | Poorly | T2N0M0 | ++ |
| D9 | 47 | F | Adenocarcinoma | Moderately | T2N0M0 | − |
| D10 | 71 | M | Squamous cell carcinoma | Moderately | T2N0M0 | + |
| D11 | 65 | M | Squamous cell carcinoma | Moderately | T2N0M0 | ++ |
| E1 | 68 | M | Adenocarcinoma, squamous cell carcinoma | Moderately | T3N0M0 | ++ |
| E2 | 47 | F | Large cell Carcinoma | Moderately | T2N0M0 | ++ |
| E3 | 39 | F | Adenocarcinoma | Moderately | T2N1M0 | − |
| E4 | 67 | M | Squamous cell carcinoma | Moderately | T2N1M0 | − |
| E5 | 60 | F | Alveolus cell carcinoma | N/A | T2N0M0 | + |
| E6 | 70 | F | Carcinoma | Moderately | T1N0M0 | + |
| E7 | 27 | M | Sarcoma, metastasis tumor | Moderately | T2NxM1 | + |
| E8 | 65 | M | Squamous cell carcinoma | Moderately | T3N0M0 | + |
| E9 | 68 | F | Squamous cell carcinoma | Moderately | T2N0M0 | + |
| E10 | 58 | F | Adenocarcinoma | Moderately | T2N1M0 | − |
| E11 | 68 | M | Squamous cell carcinoma | Well | T2N0M0 | + |
| F1 | 48 | M | Squamous cell carcinoma | Moderately | T3N0M0 | − |
| F2 | 59 | M | Squamous cell carcinoma | N/A | T1N0M0 | + |
| F3 | 54 | M | Adenocarcinoma, cyst | Moderately | T2N1M0 | + |
| F4 | 45 | M | Squamous cell carcinoma | Moderately | T3N0M0 | ++ |
| F5 | 69 | M | Squamous cell carcinoma | Poorly | T2N1M0 | ++ |
| F6 | 78 | F | Alveolus cell adenocarcinoma | Moderately | T1N0M0 | ++ |
| F7 | 60 | M | Adenocarcinoma | Moderately | T1N0M0 | + |
| F8 | 54 | F | Alveolus cell carcinoma | Moderately | T2N1M0 | − |
| F9 | 78 | M | Alveolus cell carcinoma | Moderately | T1N0M0 | − |
| F10 | 70 | M | Alveolus cell carcinoma | Well | T1N0M0 | ++ |
| F11 | 45 | F | Bronchio alveolar carcinoma | Moderately | T2N0M0 | + |

(−) negative expression
(+) low or moderate expression
(++) high expression

TABLE 9
Association between WHSC1-positivity in NSCLC and
patients' characteristics (n = 328)

| | | WHSC1 | WHSC1 | |
|---|---|---|---|---|
| | Total | positive | negative | P-value |
| | n = 328 | n = 174 | n = 154 | positive vs negative |
| Gender | | | | |
| Male | 231 | 124 | 107 | NS (0.8085) |
| Female | 97 | 50 | 47 | |
| Age(years) | | | | |
| <65 | 146 | 77 | 69 | NS (>0.9999) |
| ≧65 | 182 | 97 | 85 | |
| Histological type | | | | |
| ADC | 195 | 98 | 97 | NS (0.2599*) |
| SCC | 99 | 55 | 44 | |
| Others | 34 | 21 | 13 | |
| Smoking status | | | | |
| Never | 92 | 49 | 43 | NS (>0.9999) |
| Smoker | 236 | 125 | 111 | |
| pT factor | | | | |
| T1 | 136 | 75 | 61 | NS (0.5748) |
| T2 + T3 | 192 | 99 | 93 | |
| pN factor | | | | |
| N0 | 216 | 109 | 107 | NS (0.2015) |
| N1 + N2 | 112 | 65 | 47 | |

Abbreviation:
ADC, adenocarcinoma;
SCC, squamous-cell carcinoma;
Others, large-cell carcinoma(LCC) plus adenosquamous-cell carcinoma(ASC)
*ADC versus non-ADC
NS, no significance

TABLE 10
Cox's proportional hazards model analysis of prognostic factors in patients with NSCLCs

| | Variables | Hazards ratio | 95% CI | Unfavorable/Favorable | P-value |
|---|---|---|---|---|---|
| Univariate analysis | WHSC1 | 0.971 | 0.694-1.358 | Positive/Negative | NS (0.8629) |
| | Age(years) | 1.863 | 1.304-2.661 | 65≧/<65 | 0.0006* |
| | Gender | 1.634 | 1.100-2.427 | Male/Female | 0.0149* |
| | Histological type | 1.548 | 1.108-2.162 | nonADC/ADC | 0.0104* |
| | Smoking status | 1.312 | 0.887-1.941 | Smoker/Never | NS (0.1738) |
| | pT factor | 2.421 | 1.647-3.559 | T2 + T3/T1 | <0.0001* |
| | pN factor | 3.268 | 2.309-4.608 | N1 + N2/N0 | <0.0001* |
| Multivariate analysis | Age(years) | 2.091 | 1.454-3.007 | 65≦/<65 | <0.0001* |
| | Gender | 1.294 | 0.833-2.012 | Male/Female | NS (0.2519) |
| | Histological type | 0.935 | 0.642-1.361 | nonADC/ADC | NS (0.7247) |
| | pT factor | 1.838 | 1.220-2.770 | T2 + T3/T1 | 0.0036* |
| | pN factor | 2.227 | 1.572-3.155 | N1 + N2/N0 | <0.0001* |

Abbreviation:
ADC, adenocarcinoma
*P < 0.05
NS, no significance

### Example 4

### WHSC1 and WHSC1L1 Regulates the Growth of Cancer Cells

To examine whether elevated expression of WHSC1 and WHSC1L1 plays some critical roles in the proliferation of cancer cells, the present inventors prepared siRNA oligonucleotide duplexes, which specifically suppressed the expression of WHSC1 (siWHSC1#1, #2) and WHSC1L1 (siWHSC1L1#1, #2) and transfected each of them into cancer cells. Expression levels of WHSC1 and WHSC1L1 in various types of cancer cells using quantitative real-time PCR were examined and the results confirmed that these genes were abundantly expressed in various types of cancer cells (FIG. 7). As shown in FIG. 4A, each siRNA effectively downregulated WHSC1 and WHSC1L1 expression, compared with siEGFP and siNC controls. The effects of siRNAs on the growth of cancer cells were subsequently examined by the cell counting kit system (FIG. 4B) and found that transfection of two independent siWHSC1s and siWHSC1L1s into two bladder cancer cell lines and three lung cancer cell lines significantly suppressed their growth, compared with those with siEGFP or siNC. Also, BrdU and 7-AAD staining was performed to analyze the detailed cell cycle status of cancer cells, and confirmed that the proportion of cancer cells at the S phase was significantly reduced after the knockdown of WHSC1 (FIG. 4C) and that in G2/M phase was increased, indicating that knockdown of WHSC1 could induce G2/M arrest. These results reveal that WHSC1 and WHSC1L1 play a critical role in the growth regulation of cancer cells, and WHSC1 is an essential factor for G2/M transition.

### Example 5

### WHSC1 can Contribute to Carcinogenesis Through the Regulation of the Wnt Cascade, JNK Cascade, MAP Kinase Cascade, Cell Cycle and DNA Replication

To identify signal pathways downstream to WHSC1, the present inventors performed microarray expression analysis. After knocking down of WHSC1 in SW780 and A549 cancer cells, the present inventors isolated total RNA from SW780 and A549 24 h after the treatment with siWHSC1#1. The expression profiles of these cells were compared to the cells treated with control siRNAs (siEGFP and siFFLuc) using Affymetrix's HG-U133 Plus 2.0 Array. Expression of 74 genes decreased and 1 gene increased statistically by the knockdown of WHSC1, so these 75 genes were suggested to be the downstream genes affected by knockdown of WHSC1 (FIG. 5A, Table 11). Reproducability was shown as the present inventors were able to validate the down-regulation of several randomly selected downstream gene candidates.

Signal pathway analysis for determining the downstream candidates using the Gene Ontology database (Methods; Table 11) indicated that WHSC1 could regulate the Wnt cascade, JNK cascade, MAP kinase cascade, cell cycle and DNA replication. Therefore, dysfunction of WHSC1 expression is likely to contribute to human carcinogenesis partially through regulating these pathways.

TABLE 11
Output ratio affected by knockdown of WHSC1

| GeneSymbol | Ratio |
|---|---|
| EGR1 | 2.351 |
| HSP90AA1 | 0.813 |
| SCD | 0.781 |
| TOP1 | 0.715 |
| MMD | 0.712 |
| SACS | 0.706 |
| LBR | 0.7 |
| CKAP4 | 0.694 |
| MAP3K5 | 0.684 |
| ANKRD10 | 0.669 |
| DLD) | 0.664 |
| ANKRD57 | 0.662 |
| CCNYL1 | 0.66 |
| MDFIC | 0.659 |
| PLEKHA1 | 0.657 |
| FNIP1 | 0.657 |
| ZADH2 | 0.656 |
| IPO7 | 0.652 |
| MYO6 | 0.651 |
| RASEF | 0.651 |
| RAPGEF2 | 0.644 |
| SLC11A2 | 0.636 |
| PALM2-AKAP2 | 0.634 |
| THRB | 0.631 |
| MGAT4A | 0.631 |
| UEVLD | 0.63 |
| THBS1 | 0.629 |
| ZMYM2 | 0.629 |
| LPP | 0.628 |
| WDR68 | 0.621 |
| FAM32A | 0.616 |
| ASAP1 | 0.616 |
| CSNK1G1 | 0.614 |
| ARF3 | 0.613 |
| UNKL | 0.61 |
| IFNAR1 | 0.606 |
| ZDHHC23 | 0.606 |
| CCDC68 | 0.601 |
| MAK16 | 0.601 |
| BMP2 | 0.6 |
| KBTBD8 | 0.596 |
| MREG | 0.584 |
| TMEM41B | 0.572 |
| TNRC6B | 0.562 |
| KLHL5 | 0.562 |
| PKIB | 0.56 |
| MYBL1 | 0.559 |
| ENC1 | 0.559 |
| TRIM23 | 0.557 |
| EIF2C2 | 0.547 |
| AGPAT9 | 0.545 |
| DYNC1LI2 | 0.538 |
| LARP6 | 0.533 |
| USP46 | 0.53 |
| NUPL1 | 0.528 |
| KATNAL1 | 0.526 |
| LASS6 | 0.526 |
| ACLY | 0.523 |
| MFAP3L | 0.514 |
| NAB1 | 0.5 |
| UTP14C | 0.497 |
| ALG10B | 0.495 |
| SRI | 0.487 |
| FUBP1 | 0.485 |
| RBM7 | 0.484 |
| CPEB2 | 0.474 |
| TMEM65 | 0.456 |
| PAFAH1B2 | 0.451 |
| REEP5 | 0.439 |
| DICER1 | 0.438 |
| KCTD9 | 0.388 |
| HPS5 | 0.374 |
| MFSD6 | 0.356 |
| WHSC1 | 0.291 |
| SPG20 | 0.26 |

### Example 6

### WHSC1 can Interact with IQGAP1, TIAM1, AKT2 and Beta-Catenin

To identify protein interactions, the present inventors next performed immunoprecipitation-mass spectrometry (IP-MS) analysis and found IQGAP1, TIAM1 and AKT2 as interacting with WHSC1 (FIG. 5B). The present inventors further performed a co-immunoprecipitation assay using specific antibodies and validated each interaction (FIG. 5C). Since IQGAP1 and TIAM1 are involved in the Wnt signaling pathway through interaction with beta-catenin protein, the present inventors considered a possibility of interaction between WHSC1 and beta-catenin, and confirmed their interaction as shown in FIG. 5C. Interestingly, immunoprecipitation analysis after the nuclear/cytoplasmic fractionation showed that the interaction between WHSC1 and beta-catenin was observed specifically in the nuclear fraction (FIG. 5D). Furthermore, the present inventors confirmed co-localization of WHSC1 and beta-catenin were co-localized in the nucleus (FIG. 5E), indicating that they work cooperatively in the nucleus.

The present inventors then applied TOPFLASH and FOPFLASH reporter analyses and detected that overexpression of WHSC1 could significantly enhance TOPFLASH reporter activity (FIG. 6A), indicating that WHSC1 can positively regulate beta-catenin/Tcf-4 activity. Moreover, the present inventors also confirmed that expression of CCND1, an important downstream gene of beta-catenin/Tcf-4 complex, decreased after treatment with siWHSC1 by microarray data (FIG. 6B), and the data were also validated by real-time PCR analysis (FIG. 6C). Importantly, signal pathway analysis for determining the downstream candidates using the Gene Ontology database (Methods; Table 12) indicated that WHSC1 had the potential to regulate the Wnt signaling pathway in addition to MAP kinase and JNK pathways.

Next, chromatin immunoprecipitation (ChIP) analysis using 5 different primers targeting promoter regions of CCND1 gene was performed. As shown in FIG. 6B, both wild-type and enzyme-dead WHSC1 totally bond to the regions, and particularly showed the strong association with the location near the transcriptional start site (FIG. 11). Enzyme dead-WHSC1 (WHSC1[delta]SET) tended to show a weaker association than wild-type WHSC1 (WHSC1 wt). Meanwhile, H3K36me3 levels in the promoter region of CCND1 gene were significantly increased after transfection with wild-type WHSC1, whereas no elevation was observed in the case of WHSC1[delta]SET transfection. H3K36me3 status was likely to correlate with the status of wild-type WHSC1 accumulation. These results suggest that WHSC1 can associate with the promoter region of CCND1 and tri-methylate histone H3 lysine 36 directly. In consequence, CCND1 expression is transcriptionally activated. Furthermore, TOPFLASH and FOPFLASH reporter analyses were applied and detected that overexpression of wild-type WHSC1 significantly enhanced TOPFLASH reporter activity (FIG. 12), whereas enzyme-dead WHSC1 couldn't show the activity, indicating that WHSC1 can positively regulate beta-catenin/Tcf-4 activity. These results show that WHSC1 regulates the Wnt signaling pathway through interacting with beta-catenin, and point to the mechanisms of how WHSC1 contributes to human carcinogenesis.

TABLE 12
Gene Ontology pathway analysis based on the Affymetrix's microarray data

| Entry ID | Name | Definition | P |
|---|---|---|---|
| WHSC1 | | | |
| GO0006261 | DNA-dependent DNA | The process whereby new strands of DNA are synthesized, using parental DNA as a | 1.75 × 10⁻⁴ |
| | replication | template for the DNA-dependent DNA polymerases that synthesize the new strands. | |
| GO0007254 | JNK cascade | A cascade of protein kinase activities, culminating in the phosphorylation and activation | 5.28 × 10⁻⁴ |
| | | of a member of the JUN kinase subfamily of stress-activated protein kinases, which | |
| | | in turn are a subfamily of mitogen-activated protein (MAP) kinases that is activated | |
| | | primarily by cytokines and exposure to environmental stress. | |
| GO0043506 | Regulation of JNK | Any process that modulates the frequency, rate or extent of JUN kinase activity. | 9.44 × 10⁻⁴ |
| | activity | | |
| GO0000165 | MAPKKK cascade | Cascade of at least three protein kinase activities culminating in the phosphorylation and | 3.29 × 10⁻³ |
| | | activation of a MAP kinase. | |
| GO0006268 | DNA unwinding during | The process by which interchain hydrogen bonds between two strands of DNA are broken | 3.80 × 10⁻³ |
| | replication | or ‘melted’, generating unpaired template strands for DNA replication. | |
| GO0032508 | DNA duplex unwinding | The process by which interchain hydrogen bonds between two strands of DNA are broken | 5.19 × 10⁻³ |
| | | or ‘melted’, generating a region of unpaired single strands. | |
| GO0006270 | DNA replication initiation | The process by which DNA replication is started; this involves the separation of a stretch of | 7.64 × 10⁻³ |
| | | the DNA double helix, the recruitment of DNA polymerases and the initiation of | |
| | | polymerase action. | |
| GO0017147 | Wnt-protein binding | Interacting selectively with Wnt-protein, a secreted growth factor involved in signaling. | 9.27 × 10⁻³ |
| GO0000187 | Activation of MAPK activity | The process of formation of a ring composed of actin, myosin, and associated proteins that | 1.10 × 10⁻² |
| | | will function in cytokinesis. | |
| GO0006260 | DNA replication | The process whereby new strands of DNA are synthesized. The template for replication | 1.44 × 10⁻² |
| | | can either be an existing DNA molecule or RNA. | |
| GO0022402 | Cell cycle process | A cellular process that is involved in the progression of biochemical and morphological | 1.99 × 10⁻² |
| | | phases and events that occur in a cell during successive cell replication or nuclear | |
| | | replication events. | |
| GO0022616 | DNA strand elongation | The DNA metabolic process by which a DNA strand is synthesized by adding nucleotides | 2.33 × 10⁻² |
| | | to the 3′ end of an existing DNA stand. | |
| GO0042813 | Wnt receptor activity | Combining with a member of the Wnt family of signaling molecules to initiate a change | 2.45 × 10⁻² |
| | | in cell activity. | |
| WHSC1L1 | | | |
| GO0000186 | Activation of MAPKK | The initiation of the activity of the inactive enzyme MAP kinase kinase by phosphorylation | 3.17 × 10⁻⁴ |
| | activity | by a MAPKKK. | |
| GO0001952 | Regulation of cell-matrix | Any process that modulates the frequency, rate or extent of attachment of a cell to the | 7.32 × 10⁻⁴ |
| | adhesion | extracellular matrix. | |
| GO0051301 | Cell division | The process resulting in the physical partitioning and separation of a cell into daughter cells. | 5.39 × 10⁻³ |
| GO0016568 | Chromatin modification | The alteration of DNA or protein in chromatin, which may result in changing the | 5.69 × 10⁻³ |
| | | chromatin structure. | |
| GO0000165 | MAPKKK cascade | Cascade of at least three protein kinase activities culminating in the phosphorylation and | 9.98 × 10⁻³ |
| | nucleus | activation of a MAP kinase. MAPKKK cascadeslie downstream of numerous | |
| | | signaling pathways. | |
| GO0007049 | Cell cycle | The progression of biochemical and morphological phases and events that occur in a cell | 1.11 × 10⁻² |
| | | during successive cell replication or nuclear replication events. Canonically, the cell cycle | |
| | | comprises the replication and segregation of genetic material followed by the division of | |
| | | the cell, but in endocycles or syncytial cells nuclear replication or nuclear division may | |
| | | not be followed by cell division. | |
| GO0043405 | Regulation of MAP | Any process that modulates the frequency, rate or extent of MAP kinase activity. | 1.35 × 10⁻² |
| | kinase activity | | |

Discussion

WHSC1, a histone lysine methyltransferase, is demonstrated herein to be upregulated in several cancer types and likely to have a critical role in the growth regulation of cancer cells through the regulation of the Wnt signaling pathway. WHSC1 is a member of a gene family that currently includes two additional members: nuclear receptor-binding SET domain protein 1 (NSD1) and WHSC1L1, both of which show 70-75% sequence identity with WHSC1. In AML, the recurring t(5;11)(q35;p15.5) translocation fuses NSD1 to nucleoporin 98 (NUP98) (Cerveira N, et al. Leukemia 2003; 17:2244-7.). NUP98-NSD1 was shown to induce AML in vivo and sustain self-renewal of myeloid stem cells in vitro (Wang G G, et al. Nat Cell Biol 2007; 9:804-12.). Mechanistically, the NUP98-NSD1 complex binds genomic elements adjacent to HoxA7 and HoxA9, and maintains EZH2-mediated transcriptional repression of the Hox-A locus during differentiation through regulation of histone H3 Lys 36 (H3K36) methylation and histone acetylation (Wang G G, et al. Nat Cell Biol 2007; 9:804-12.).

Importantly, either deletion of the NUP98 FG-repeat domain or mutations in NSD1 that led to inactivation of the methyltransferase activity, precluded both Hox-A gene activation and myeloid progenitor immortalization, indicating that the methyltransferase activity of NSD1 is likely to play a critical role in tumorigenesis. In addition to NSD1, we found that WHSC1L1 was overexpressed in several tumors and dysregulation of its expression could be involved in human carcinogenesis. These data indicate that abnormal expression of a family of histone methyltransferases (NSD1, WHSC1 and WHSC1L1) are important in human carcinogenesis.

IQGAP1 is a 190-kDa protein that contains multiple protein-interacting domains and stoichiometrically binds to beta-catenin (Kuroda S, et al. Science 1998; 281:832-5.). Overexpression of IQGAP1 in SW480 colon carcinoma cells increased the amount of beta-catenin in the nucleus and enhanced beta-catenin-mediated transcriptional activation (Briggs M W, et al. J Biol Chem 2002; 277:7453-65.). The disparate effects of IQGAP1 on beta-catenin function cooperate to increase both the proliferative capacity (by enhancing transcription of TCF/LEF-regulated promoters) and metastatic potential (by reducing cell-cell adhesion) of malignant cells (Briggs M W, et al. J Biol Chem 2002; 277:7453-65.). On the other hand, overexpression of the TIAM1 (T-cell lymphoma invasion and metastasis-inducing protein 1) was found in highly invasive breast tumors (Adam L, et al. J Biol Chem 2001; 276:28443-50.) and colon carcinomas (Liu L, et al. World J Gastroenterol 2005; 11:705-7.; Minard M E, et al. Clin Exp Metastasis 2006; 23:301-13.). Upon Wnt pathway stimulation, beta-catenin may form a complex with TIAM1, which is recruited to the promoters in the Wnt target genes by a promoter-associated complex containing TCF/LEF and inactive, GDP-bound Rac1. Then, TIAM1 activates Rac 1 by catalyzing GDP to GTP exchange, and thereby mediates the stimulatory effects of Rac1 on the Wnt-induced transcription factor complex. This results in the enhanced transcription of a subset of Wnt target genes that include those promoting unrestricted cell proliferation like CCND1(Buongiorno P, et al. Mol Cancer 2008; 7:73.). In this study, we found that WHSC1 could interact with IQGAP1, TIAM1 and beta-catenin, and the series of experiments imply that WHSC1 can regulate the Wnt signaling pathway in cancer cells. It has been reported that dysregulation of the Wnt signaling pathway is involved in many human cancers, including lung and bladder cancers (Minna J D, et al. Cancer Cell 2002; 1:49-52.; Yue W, Sun Q, Dacic S, et al. Carcinogenesis 2008; 29:84-92.; Thievessen I, et al. Br J Cancer 2003; 88:1932-8.). Taken together, the data disclosed herein indicate that WHSC1 is overexpressed in wide range of human cancers, indicating that the WHSC1-dependent dysregulation mechanism of the Wnt signaling pathway is one of the important factors in human carcinogenesis.

The expression analysis showed that expression levels of WHSC1 and WHSC1L1 in normal tissues are very low (FIG. 8).The BioGPS database also revealed that expression of these genes in many types of tissues is very low (FIG. 9). As expression levels of WHSC1 and WHSC1L1 in various types of cancer are significantly higher than those in corresponding non-neoplastic tissues, WHSC1 and WHSC1L1 are promising targets for development of novel cancer therapies. Furthermore, since knockdown of either WHSC1 or WHSC1L1 suppressed the growth of several cancer cells, these enzymes appear to have a critical role in the growth regulation of cancer cells. The data indicate that an inhibitor(s) for WHSC1 and WHSC1L1 is an ideal candidate for molecular targeted therapy of cancer.

The wingless/int (Wnt) signaling pathway regulates cellular proliferation and differentiation in vertebrates and invertebrates. Beta-catenin is a double-functional molecule in the Wnt signaling pathway and the E-cadherin-catenin complex. When it accumulates in the nucleus, beta-catenin loses its function as a cell-adhesion molecule, which activates the Wnt signaling pathway and switches on transcription of target genes such as CCND1. It has been reported that dysregulation of the Wnt signaling pathway is involved in many human cancers, including bladder and lung cancers (33-37 Mazieres J, et al. Cancer Lett 2005; 222:1-10., Minna J D, et at. Cancer Cell 2002; 1:49-52., Thievessen I, et al. Br J Cancer 2003; 88:1932-8., Urakami S, et al. Clin Cancer Res 2006; 12:383-91., Yue W, et al. Carcinogenesis 2008; 29:84-92.). Mutations that promote constitutive activation of the Wnt signaling pathway lead to cancer. The best-known example is Familial Adenomatous Polyposis (FAP), an autosomal, dominantly inherited disease in which patients display polyps in the colon and rectum. This disease is caused most frequently by truncations in APC (Nishisho I, et al. Science 1991; 253:665-9.) that promote aberrant activation of the Wnt pathway leading to adnomatous lesions due to increased cell proliferation. Mutations in beta-catenin have also been found in sporadic colon cancers (Giles R H, et al. Biochim Biophys Acta 2003; 1653:1-24.). On the contrary, although the dysregulation of Wnt signaling in various types of cancer has been implied (Nusse R. Cell Res 2005; 15:28-32. Paul S, et al. Neoplasma 2008; 55:165-76.), such mutations seem to be rare in a number of cancers including bladder and lung carcinomas (Mazieres J, et al. Cancer Lett 2005; 222:1-10., Ohgaki H, et al. Cancer Lett 2004; 207:197-203., Ueda M, et al. Br J Cancer 2001; 85:64-8.). The facts indicate that several other factors may also regulate the Wnt pathway in human carcinogenesis.

In this study, it was found that WHSC1 could interact with beta-catenin in the nucleus and promote tri-methylation of histone H3 at lysine 36 (H3-K36) in the promoter region of CCND1 (FIG. 13). Generally, methylated H3-K36 is enriched in regions of active transcription (Pokholok D K, et al. Cell 2005; 122:517-27.), and it has also been linked to transcriptional elongation (Xiao T, et al. Genes Dev 2003; 17:654-63.) and alternative splicing (Luco R F, et al. Science; 327:996-1000.). In AML, the recurring t(5;11)(q35;p15.5) translocation fuses NSD1, the family gene of WHSC1, to nucleoporin 98 (NUP98) (Cerveira N, et al. Leukemia 2003; 17:2244-7.). NUP98-NSD1 was shown to induce AML in vivo and sustain self-renewal of myeloid stem cells in vitro (Wang G G, et al. Nat Cell Biol 2007; 9:804-12.). Mechanistically, the NUP98-NSD1 complex binds genomic elements adjacent to HoxA7 and HoxA9, maintains H3-K36 tri-methylation, and prevents transcriptional repression of the HoxA locus. Importantly, either deletion of the NUP98 FG-repeat domain or mutations in NSD1 that led to inactivation of the methyltransferase activity, precluded both Hox-A gene activation and myeloid progenitor immortalization, indicating that NSD1-dependent H3-K36 methylation is likely to play a critical role in tumorigenesis. Consistent with this, it was demonstrated that WHSC1 cooperatively enforces the transcriptional activity of beta-catenin through maintaining H3-K36 tri-methylation. This implies that WHSC1-dependent H3-K36 methylation may promote tumorigenesis in a synergistic manner together with beta-catenin, and a novel mechanism of the Wnt pathway dysregulation in human carcinogenesis through the epigenetic regulation is presented.

The expression analysis showed that beta-catenin is abundantly expressed in bladder and lung censer cell lines as well as the human colon cancer cell line HCT116 (FIG. 14A). To elucidate the significance of beta-catenin in the growth regulation of cancer cells, we examined knockdown experiments using specific siRNAs targeting beta-catenin (FIGS. 14B and C). The growth rate of bladder and lung cancer cells was significantly suppressed after knockdown of beta-catenin, indicating that the Wnt/beta-catenin pathway may play an important role in the growth regulation of these cells. Because WHSC1 is overexpressed in various types of cancers like pancreatic and breast cancers besides bladder and lung cancers, it is possible that the dysregulation of Wnt/beta-catenin pathway presented in this study may be observed in other cancers. Intriguingly, flow cytometric cell cycle analysis revealed that knockdown of WHSC1 reduced the cell population of cancer cells at S phase and increased that at G2/M phase. Indeed, evidence has been accumulated that components of the WNT/beta-catenin pathway including beta-catenin localize to the mitotic spindle or centrosomes and are involved in the regulation of mitotic progression (Bahmanyar S et al. Genes Dev 2008; 22:91-105. Hadjihannas M V, et al. Proc Natl Acad Sci USA 2006; 103:10747-52.), indicating that WHSC1 might also regulate the M phase of cancer cells through interacting with the Wnt/beta-catenin pathway.

Furthermore, in vitro methyltransferase assay was performed to validate the possibility that beta-catenin serves as a substrate of WHSC1-dependent methylation, but no positive signals were observed (data not shown). Therefore, the transcriptional regulation of beta-catenin by the methylation activity of WHSC1 appears to be based on the H3-K36 methylation at the moment.

As mentioned above, NSD1 was reported to promote tumorigenesis in AML (Wang G G, et al. Nat Cell Biol 2007; 9:804-12.), and it was identified that expression levels of WHSC1 and WHSC1L1 in various types of cancer are significantly higher than those in corresponding non-neoplastic tissues. According to these data, the present inventor propose that abnormal expression of a family of methyltransferases (NSD1, WHSC1 and WHSC1L1) may be important in human carcinogenesis. Especially, as the expression analysis showed that expression levels of WHSC1 and WHSC1L1 in normal tissues are significantly low (FIG. 8) and the BioGPS database also revealed that expression of these genes in many types of tissues is very low (FIG. 9), WHSC1 and WHSC1L1 are likely to be promising targets for development of novel cancer therapies. Since knockdown of either WHSC1 or WHSC1L1 suppressed the growth of several cancer cells, these enzymes appear to have a critical role in the growth regulation of cancer cells. The data imply that an inhibitor(s) for WHSC1 and WHSC1L1 may be an ideal candidate for molecular targeted therapy of cancer. As the development of methyltransferase inhibitors has started just recently (Greiner D, et al. Nat Chem Biol 2005; 1:143-5., Kubicek S, et al. Mol Cell 2007; 25:473-81.), further studies may ensure the usefulness of this approach in the near future.

### INDUSTRIAL APPLICABILITY

The present inventors have shown that cancer cell growth is suppressed by a doublestranded nucleic acid molecule that specifically targets the WHSC1 or WHSC1L1 gene. Thus, the double-stranded nucleic acid molecule is useful for anti-cancer pharmaceuticals. Agents that block the expression of WHSC1 or WHSC1L1 protein or prevent its activity can find therapeutic utility as anti-cancer agents, particularly anti-cancer agents for the treatment of bladder cancer, breast cancer, cholangiocellular carcinoma, CML, esophageal cancer, HCC, NSCLC, SCLC, osteosarcoma, pancreatic cancer, prostate cancer, renal cell carcinoma, soft tissue tumor or lymphoma.

While the present invention has been described in detail and with reference to specific embodiments thereof, it will be apparent to one skilled in the art that various changes and modifications can be made therein without departing from the spirit and scope of the invention. All publications, patent applications, patents, and other references mentioned herein are incorporated by reference in their entirety. In case of conflict, the present specification, including definitions, will control. In addition, the materials, methods, and examples are illustrative only and not intended to be limiting.

1.-5. (canceled)

6. A method of screening for a candidate substance for treating or preventing cancer, or inhibiting cancer cell growth, said method comprising steps of:
(a) contacting a test substance with a polypeptide encoded by a WHSC1 or WHSC1L1 gene;
(b) detecting a binding activity between the polypeptide and the test substance; and
(c) selecting the test substance that binds to the polypeptide.

7. A method of screening for a candidate substance for treating or preventing cancer, or inhibiting cancer cell growth, said method comprising steps of:
(a) contacting a test substance with a cell expressing either or both of a WHSC1 and WHSC1L1 gene;
(b) detecting either of the expression level of the WHSC1 or the expression level of the WHSC1L1 gene, or both; and
(b) selecting the test substance that reduces either of the expression level of the WHSC1 gene or the expression level of the WHSC1L1 gene, or both in comparison with the expression level detected in the absence of the test substance.

8. A method of screening for a candidate substance for treating or preventing cancer, or inhibiting cancer cell growth, said method comprising the steps of:
(a) contacting a test substance with a polypeptide encoded by a WHSC1 or WHSC1L1 gene;
(b) detecting a biological activity of the polypeptide of step (a); and
(c) selecting the test substance that suppresses the biological activity of the polypeptide in comparison with the biological activity detected in the absence of the test substance.

9. The method of claim 8, wherein the biological activity is cell proliferative activity, methyltransferase activity or binding activity to an IQGAP1 polypeptide, TIAM1 polypeptide, AKT2 polypeptide or beta-catenin polypeptide.

10. A method of screening for a candidate substance for treating or preventing cancer, or inhibiting cancer cell growth, said method comprising the steps of:
(a) contacting a test substance with a cell into which a vector comprising a transcriptional regulatory region of WHSC1 or WHSC1L1 gene and a reporter gene that is expressed under control of the transcriptional regulatory region has been introduced,
(b) measuring expression or activity of said reporter gene; and
(c) selecting the test substance that reduces the expression or activity of said reporter gene, as compared to the expression or activity in the absence of the test substance.

11. A method of screening for a candidate substance for treating or preventing cancer, or inhibiting cancer cell growth, said method comprising the steps of:
(a) contacting at least one of the polypeptides selected from the group consisting of an IQGAP1 polypeptide, TIAM1 polypeptide, AKT2 polypeptide and beta-catenin polypeptide, or functional equivalent thereof with a WHSC1 polypeptide or functional equivalent thereof in the presence of a test substance;
(b) detecting the binding between at least one of polypeptides selected from the group consisting of the IQGAP1 polypeptide, TIAM1 polypeptide, AKT2 polypeptide and beta-catenin polypeptide, and the WHSC1 polypeptide of step (a); and
(c) selecting the test substance that inhibits the binding between at least one of the polypeptides selected from the group consisting of the IQGAP1 polypeptide, TIAM1 polypeptide, AKT2 polypeptide and beta-catenin polypeptide, and the WHSC1 polypeptide as compared to the binding detected in the absence of the test substance.

12. A double-stranded molecule comprising a sense strand and an antisense strand, wherein the sense strand comprises a nucleotide sequence corresponding to a target sequence selected from the group consisting of SEQ ID NO: 29, 32, 35 and 38, and wherein the antisense strand comprises a nucleotide sequence which is complementary to the target sequence, wherein said sense strand and said antisense strand hybridize to each other to form said double-stranded molecule, and wherein said double-stranded molecule, when introduced into a cell expressing the WHSC1 or WHSC1L1 gene, inhibits expression of said gene.

13. The double-stranded molecule of claim 12, wherein the doublestranded molecule is between about 19 and about 25 nucleotides in length.

14. The double-stranded molecule of claim 12, wherein said double-stranded molecule is a single polynucleotide molecule comprising the sense strand and the antisense strand linked via a single-stranded nucleotide sequence.

15. The double-stranded molecule of claim 14, wherein said polynucleotide has the general formula of
5′-[A]-[B]-[A′]-3′,
wherein [A] is a sense strand comprising a nucleotide sequence corresponding to a target sequence selected from the group consisting of SEQ ID NO: 29, 32, 35 and 38; [B] is a nucleotide sequence consisting of about 3 to about 23 nucleotides; and [A′] is an antisense strand comprising a nucleotide sequence complementary to the target sequence.

16. A vector encoding the double-stranded molecule of claim 12.

17. A vector comprising each of a combination of polynucleotide comprising a sense strand nucleic acid and an antisense strand nucleic acid, wherein said sense strand nucleic acid comprises a nucleotide sequence selected from the group consisting of SEQ ID NOs: 29, 32, 35 and 38, and said antisense strand nucleic acid consists of a sequence complementary to the sense strand, wherein the transcripts of said sense strand and said antisense strand hybridize to each other to form a double-stranded molecule, and wherein said vector inhibits expression of a target gene.

18. A method of treating or preventing cancer in a subject, comprising administering to said subject a pharmaceutically effective amount of a doublestranded molecule directed against a WHSC1 or WHSC1L1 gene, or a vector encoding said double-stranded molecule, and a pharmaceutically acceptable carrier, wherein the doublestranded molecule inhibits cell proliferation and the expression of the WHSC1 or WHSC1L1 gene when introduced into a cell expressing the WHSC1 or WHSC1L1 gene.

19. The method of claim 18, wherein the doublestranded molecule is that of claim 12.

20. The method of claim 18, wherein the vector is that of claim 16.

21. A composition for treating or preventing cancer, which comprises a pharmaceutically effective amount of a double-stranded molecule directed against a WHSC1 or WHSC1L1 gene, or a vector encoding said double-stranded molecule, and a pharmaceutically acceptable carrier, wherein the double-stranded molecule inhibits cell proliferation and expression of the WHSC1 or WHSC1L1 gene when introduced into a cell expressing the WHSC1 or WHSC1L1 gene.

22. The composition of claim 21, wherein the doublestranded molecule is that of claim 12.

23. The composition of claim 21, wherein the vector is that of claim 16.

24. A method of screening for a substance for inhibiting the binding between WHSC1 polypeptide or functional equivalent thereof and at least one of the polypeptides selected from the group consisting of an IQGAP1 polypeptide, TIAM1 polypeptide, AKT2 polypeptide and beta-catenin polypeptide, or functional equivalent thereof, said method comprising the steps of:
(a) contacting at least one of the polypeptides of an IQGAP1 polypeptide, TIAM1 polypeptide, AKT2 polypeptide and beta-catenin polypeptide, or functional equivalent thereof with a WHSC1 polypeptide or functional equivalent thereof in the presence of a test substance;
(b) detecting the binding between at least one of the polypeptides selected from the group consisting of the IQGAP1 polypeptide, TIAM1 polypeptide, AKT2 polypeptide and beta-catenin polypeptide, or functional equivalent, and the WHSC1 polypeptide or the functional equivalent thereof of step (a); and
(c) selecting the test substance that inhibits the binding between at least one of the polypeptides selected from the group consisting of the IQGAP1 polypeptide, TIAM1 polypeptide, AKT2 polypeptide and beta-catenin polypeptide, or functional equivalent thereof, and the WHSC1 polypeptide or the functional equivalent thereof.


SEQUENCE LISTING (62 sequences)

SEQ ID NO: 1 - DNA, 7589 bp, Homo sapiens
gccgcgcgcgagagcctcggcctggccgcgctgcgcccgccgccgccgccgccccctccccgcctgggccctaccgccgcacggccccggccccctcccagcctgccgctccggagagccgcccgccgaggatgcgacgcaccgcagtgttctaagaacggaagcatctgggctggatggaatttagcatcaagcagagtcccctttctgttcagagtgttgtaaagtgcataaagatgaagcaggcaccagaaatcctcggcagtgccaacgggaagactccgagctgcgaggtgaaccgcgagtgttctgtgttcctcagcaaagcccagctctccagtagcctgcaggagggggtcatgcagaagtttaacggccacgacgccctgccctttattccagccgacaagctgaaagatcttacttcccgggtgtttaatggagaacccggcgcacacgatgccaaactgcgttttgagtcccaggaaatgaaagggattgggacaccccctaacactacccctatcaaaaatggctctccagaaattaagctgaaaatcaccaaaacatacatgaatgggaagcctctctttgaatcttccatttgtggtgacagtgctgctgatgtgtctcagtcagaagaaaatggacaaaaaccagaaaacaaggcgagaaggaacaggaagaggagcataaaatatgactccttgctggagcagggccttgtcgaagcagctcttgtgtctaagatctcaagtccttcagataaaaagattccagctaagaaagagtcttgtccaaacactggaagagacaaagaccacctgttgaaatacaacgttggtgatttggtgtggtccaaagtgtcgggttacccttggtggccttgcatggtttctgcagatccactccttcacagctataccaaacttaaaggtcagaaaaagagtgcacgccagtatcacgtacagttctttggtgacgccccagaaagagcttggatatttgagaagagcctcgtagcttttgaaggagaaggacagtttgaaaaattatgccaggaaagtgccaagcaggcacccacgaaagctgagaaaattaagctattgaaaccaatttcagggaaattgagggcccagtgggaaatgggcattgttcaagcagaagaagctgcaagcatgtcagtggaggagcggaaagccaagttcacctttctctatgtgggggaccagcttcatctcaaccctcaagtagccaaggaggctggcattgctgcagagtctttgggagaaatggcagaatcctcaggagtcagtgaagaagctgctgaaaaccccaagtctgtgagagaagagtgcattcccatgaagagaaggcggagggccaaactgtgtagctctgcagagaccctggagagtcaccccgacatagggaagagtactcctcaaaagacggcagaggctgaccccagaagaggagtagggtctcctcctgggaggaagaagaccacagtctccatgccacgaagcaggaagggagatgcagcatcccagtttttggtcttctgtcaaaaacacagggatgaggtggtagctgagcacccagatgcttcaggtgaggagattgaagagctgctcaggtcacagtggagtctgctgagtgagaagcagagagcacgctacaacaccaagtttgccctggtggcccctgtccaggctgaagaagactctggtaatgtaaatgggaaaaaaagaaaccacacaaagaggatacaggaccctacagaagatgctgaagctgaggacacacccaggaaaagactcaggacggacaagcacagtcttcggaagagagacacaatcactgacaaaacggccagaacaagctcttacaaggccatggaggcagcctcctcgctcaagagccaggcagcaacgaaaaatctgtctgatgcatgtaaaccactgaagaagcgaaatcgggcttccacggcagcatcttcagctcttgggtttagcaaaagttcatctccttctgcatccttaactgagaatgaggtctcggacagcccgggagacgagccctcggagtccccatacgaaagtgcagacgaaacacaaactgaagtatctgtctcatccaaaaagtctgagcgaggagtgactgccaaaaaggagtatgtgtgccagctgtgtgagaagccgggcagcctcctgctctgtgaaggaccctgctgcggagctttccacctcgcctgccttgggctttcccggaggccagaagggaggttcacctgcagcgagtgtgcctcagggattcactcatgtttcgtgtgtaaagagagcaagacagatgttaagcgctgtgtggtaactcagtgtggaaaattttaccatgaggcttgtgtgaaaaaataccctctgactgtatttgagagccgaggtttccgctgccccctccacagctgtgtgagctgccatgcttccaacccttcaaacccaaggccgtcaaaaggtaaaatgatgcggtgtgtccgctgccccgttgcctatcacagcggggatgcttgtctggcagcaggatgctcagtgatcgcctccaacagcatcatctgcactgcccacttcactgctcggaaggggaagcgacaccacgcccacgtcaacgtgagctggtgcttcgtgtgctccaaaggggggagccttctgtgctgtgagtcctgcccagcggccttccaccctgactgcctgaacatcgagatgcctgacggcagctggttctgcaatgactgcagggctgggaagaagctgcacttccaggatatcatttgggtgaaacttgggaactacagatggtggccggcagaagtttgccatcccaaaaatgttcccccaaatattcagaaaatgaagcacgagattggagaattccctgtgtttttctttgggtctaaagattattactggacgcatcaggcgcgagtgttcccgtacatggagggggaccggggcagccgctaccagggggtcagagggatcggaagagtcttcaaaaacgcactgcaagaagctgaagctcgttttcgtgaaattaagcttcagagggaagcccgagaaacacaggagagcgagcgcaagcccccaccatacaagcacatcaaggtgaataagccttacgggaaagtccagatctacacagcggatatttcagaaatccctaagtgcaactgcaagcccacagatgagaatccttgtggctttgattcggagtgtctgaacaggatgctgatgtttgagtgccacccgcaggtgtgtcccgcgggcgagttctgccagaaccagtgcttcaccaagcgccagtacccagagaccaagatcatcaagacagatggcaaagggtggggcctggtcgccaagagggacatcagaaagggagaatttgttaacgagtacgttggggagctgatcgacgaggaggagtgcatggcgagaatcaagcacgcacacgagaacgacatcacccacttctacatgctcactatagacaaggaccgtataatagacgctggccccaaaggaaactactctcgatttatgaatcacagctgccagcccaactgtgagaccctcaagtggacagtgaatggggacactcgtgtgggcctgtttgccgtctgtgacattcctgcagggacggagctgacttttaactacaacctcgattgtctgggcaatgaaaaaacggtctgccggtgtggagcctccaattgcagtggattcctcggggatagaccaaagacctcgacgaccctttcatcagaggaaaagggcaaaaagaccaagaagaaaacgaggcggcgcagagcaaaaggggaagggaagaggcagtcagaggacgagtgcttccgctgcggtgatggcgggcagctggtgctgtgtgaccgcaagttctgcaccaaggcctaccacctgtcctgcctgggccttggcaagcggcccttcgggaagtgggaatgtccttggcatcattgtgacgtgtgtggcaaaccttcgacttcattttgccacctctgccccaattcgttctgtaaggagcaccaggacgggacagccttcagctgcaccccggacgggcggtcctactgctgtgagcatgacttaggggcggcatcggtcagaagcaccaagactgagaagccccccccagagccagggaagccgaaggggaagaggcggcggcggaggggctggcggagagtcacagagggcaaatagcgccaggcggccgcttggccggatccaggggcggtgcagggcggccggccctgcctgcgggagagggcgagcatgaactggcccggaggacccagctcgagccgccaggacacagacgtacaggcctcctcgggagggagcgcctccccaccactgagccatcctcagcagcgtccgctgcgtctgcactgatgaccgtctgagcccagctcagcgttcctggacaaacagcctcactcctcagcgttaccgccacacttgaatttctccgaatgtcaaggttccctcccactctatttttttaggttaaagttaattggcatatggaatgttttaatctcctctgaaatgtgtagcgtaggcttttcccaagggtcgctagaaactcgtcttcgcgttgccccctttctggctctcagcgccgtcgccactcgggagaggctgggtgaggcccgtgtgaggactgaccctggattcctcgaaactgccattgtgatcattactctgctctttggaaatggctgtatcatttttttgtactaatgtgaattgttcctcagaaacgcttcttttccatcctagtgagaagctggccctgcaggtggtggcagcaatggtgttgtaagatttcctcccgtagttttttctcctcatggatttgaatgaaatgccaataacacgtccactttcaacgtgtagtttacgcggagcactttcgaggcctggccgggttgggcctacttctcacctgggcctatcttctgaactcgctaggttcttatcaacatttgggggataactttgtatatttttttcatttggcttttctttaccagtttctgatttttattctcaatatatttttgctaaacctatttcacaaatcaccaccgactgaagtgtgtgtttactgatgcggccctgagctccatggcgaaaggagtgactttgcagggcgtgagaccgcagtctgcttagagcacaggaagtgacaacttagggagccccgtagggcgctgcaggccccggggaccccagcacgtgggtctaaagagagacggagtctagctctcctgccacccagagtggcttccatctcagcactctgtgggtctggtgatggaagatgcagtctctgctgatcacatgtgccctctgccagggcacctactgagaggtgcggtcctgggggtggaggcctgcctggcaggtgtgcgtgcctcgtacgtgtgttatgggcactggtctaggccaggtatgacacccactctcctgtgagatttcactttagtttttaaaaggtccagttctacagagtgagacctatctatctgagtactacatatgttttaagacttggttctttttttgagggatccttgaccctgggaagtctggagcaccctgagaagggggcaccatgtgtgcctttgcccacgtgtcctgaggggctgcttgtctgggagggagggagagaacattcagcagcaggtgcttttttatggccttttcttaaaataacctaagggggacacatccatcttgcagagaagtttacagaactccccttgaaaactgctgctgaggctcctgttaaattttctgtggcatcttttatgccttggtaaaaactgcagtgtctttggacctgagagtggctactccgtggttttgtgacctgtaagcgtggggttcaggggtgtgtggccctgcagggtcccacgcctccctgagcactgactggaagtttcactggctggtggctgtcccttctcccatcagggtccccagcaaagttaactacacagaggacccaggggaaacgagctgtgtagccactgacttgctcgcgcggccgtggcctctgaggggcactcgccggttaagacagggtgggagtagtgctttccagttcagactctaacttctcccaaagtgtcctaagaaaatactggatcggctcatagatttatgctccttatgatgccctaacttggaaggttgttctagggacaggccgggcagtgtccccacacacaccttagagtcgaaggccccagggccccgctgtcacttgcccaaaagatcccttccggcaggtaagggactaccaatgcttacgtcaaaacagcagaatcggctttgcagtgcactttggggagcagatattaacttatttttgtgttggacagtagtgaaatcttgtgatttttaatcgctttgataatacttccaaattttatgatttttctgaaggaaataatgcaaacattttaaatatgtttctccccctttccaaaaactgttaaactaatgagcaagtaacactaactttgaatgtctctacaatacccgttgataactcagtggagccaggctttggggtagcggccctgagcttgcagggtttctcgccactggggctgaccacgcccccagctgtgaccgtgggtgtggctggctctcggccctgcccagctttgttctgaggacgtggtgacttcctgaacatcagcttcaatcctccatcattaatgtgaagcaaaacacaaaaaccgccccaatccctcaggattccttggcatccgaaaccagcatctgcacctaaacccatacccacccgtgtgcgcccacagggggatgtgtccgaatgggcagcttaaaatgtggtcacctgtgggggaaactcttcaggcacctgaagtgagaacccagctgtccgtcctcaggccggcctttcttccggcgacacccgtccatggctggctgggtccccttcgcagtgtttgtctgtcttgacatctaaaccccggcgtgtgcagtgcccatcttccaggactaccttattttccagaattaaacctgttttataattcaagttaatgcaaatgactgtcagttgccaaatatcttgatcctatgagtgtagttgatgactgtttgttagtcagtagagtaaaatgctgtgtccacggggtgtcacagcctcaccataccctgttgaggtgtgaaatgccccgtcagaaattaaatacaaacttaaatgtgcctattggtgtctaaacttcatacaatgtaaggtcagattccttttaggaatactgggtgctgtcaccaggtttgatagttagacttaaaaacttgaaattcactttttggggggagggatatactgaaatagagagttgagacttgccagttgggggaaaatagcatttaaaatggaaagctgtgtttggaaaattgtgtatgagtatttttgtattaaaaacattttaaaggcttttttcttaacttaaaaaaaaaaaaaaaaaa

SEQ ID NO: 2 - protein, 1365 aa, Homo sapiens
MEFSIKQSPLSVQSVVKCIKMKQAPEILGSANGKTPSCEVNRECSVFLSKAQLSSSLQEGVMQKFNGHDALPFIPADKLKDLTSRVFNGEPGAHDAKLRFESQEMKGIGTPPNTTPIKNGSPEIKLKITKTYMNGKPLFESSICGDSAADVSQSEENGQKPENKARRNRKRSIKYDSLLEQGLVEAALVSKISSPSDKKIPAKKESCPNTGRDKDHLLKYNVGDLVWSKVSGYPWWPCMVSADPLLHSYTKLKGQKKSARQYHVQFFGDAPERAWIFEKSLVAFEGEGQFEKLCQESAKQAPTKAEKIKLLKPISGKLRAQWEMGIVQAEEAASMSVEERKAKFTFLYVGDQLHLNPQVAKEAGIAAESLGEMAESSGVSEEAAENPKSVREECIPMKRRRRAKLCSSAETLESHPDIGKSTPQKTAEADPRRGVGSPPGRKKTTVSMPRSRKGDAASQFLVFCQKHRDEVVAEHPDASGEEIEELLRSQWSLLSEKQRARYNTKFALVAPVQAEEDSGNVNGKKRNHTKRIQDPTEDAEAEDTPRKRLRTDKHSLRKRDTITDKTARTSSYKAMEAASSLKSQAATKNLSDACKPLKKRNRASTAASSALGFSKSSSPSASLTENEVSDSPGDEPSESPYESADETQTEVSVSSKKSERGVTAKKEYVCQLCEKPGSLLLCEGPCCGAFHLACLGLSRRPEGRFTCSECASGIHSCFVCKESKTDVKRCVVTQCGKFYHEACVKKYPLTVFESRGFRCPLHSCVSCHASNPSNPRPSKGKMMRCVRCPVAYHSGDACLAAGCSVIASNSIICTAHFTARKGKRHHAHVNVSWCFVCSKGGSLLCCESCPAAFHPDCLNIEMPDGSWFCNDCRAGKKLHFQDIIWVKLGNYRWWPAEVCHPKNVPPNIQKMKHEIGEFPVFFFGSKDYYWTHQARVFPYMEGDRGSRYQGVRGIGRVFKNALQEAEARFREIKLQREARETQESERKPPPYKHIKVNKPYGKVQIYTADISEIPKCNCKPTDENPCGFDSECLNRMLMFECHPQVCPAGEFCQNQCFTKRQYPETKIIKTDGKGWGLVAKRDIRKGEFVNEYVGELIDEEECMARIKHAHENDITHFYMLTIDKDRIIDAGPKGNYSRFMNHSCQPNCETLKWTVNGDTRVGLFAVCDIPAGTELTFNYNLDCLGNEKTVCRCGASNCSGFLGDRPKTSTTLSSEEKGKKTKKKTRRRRAKGEGKRQSEDECFRCGDGGQLVLCDRKFCTKAYHLSCLGLGKRPFGKWECPWHHCDVCGKPSTSFCHLCPNSFCKEHQDGTAFSCTPDGRSYCCEHDLGAASVRSTKTEKPPPEPGKPKGKRRRRRGWRRVTEGK

SEQ ID NO: 3 - DNA, 3995 bp, Homo sapiens
gggggctttgtgcgcggcggcggcgggagaggcggcggcggcggccagcacggaggcggaggccgagggggctgtgcacaggtcgccgcggagaggcgtgcgaattccgagccgagcgccgaggaccgtgctacccaggccgggctgccagccgcaggctcctctctggcagcagcggcggcgcggcgacccccgtccctcggcctccccttcccatcccacctcccgagccttcctcttcccgcagcacgcccggcccggcccggccgtggccctcctcagtgccggccgccatggcagaggcgtccggcgcggggaaaatctagcccggggatttcatgcggcctagctcggttccgcctcctcctcgcgcggccccagcggctgcccgcaccccagccccactccgggcctccgtgtctctcctgtgatcgcactgacacggccggggggttagaatggaacaaactgaaggcccgatgagagaaagggaaagttaaggatgctggagcagaacaatggatttctctttctctttcatgcaagggatcatgggaaacacaattcagcaaccacctcaactcattgactccgccaacatccgtcaggaggatgcctttgataacaacagtgacattgctgaagatggtggccagacaccatatgaagctactttgcagcaaggctttcagtacccagctacaacagaagatcttcctccactcacaaatgggtatccatcatcaatcagtgtgtatgaaactcaaaccaaataccagtcatataatcagtatcctaatgggtcagccaatggctttggtgcagttagaaactttagccccactgactattatcattcagaaattccaaacacaagaccacatgaaattctggaaaaaccttcccctccacagccaccacctcctccttcggtaccacaaactgtgattccaaagaagactggctcacctgaaattaaactaaaaataaccaaaactatccagaatggcagggaattgtttgagtcttccctttgtggagaccttttaaatgaagtacaggcaagtgagcacacgaaatcaaagcatgaaagcagaaaagaaaagaggaaaaaaagcaacaagcatgactcatcaagatctgaagagcgcaagtcacacaaaatccccaaattagaaccagaggaacaaaatagaccaaatgagagggttgacactgtatcagaaaaaccaagggaagaaccagtactaaaagaggaagccccagttcagccaatactatcttctgttccaacaacggaagtgtccactggtgttaagtttcaggttggcgatcttgtgtggtccaaggtgggaacctatccttggtggccttgtatggtttcaagtgatccccagcttgaggttcatactaaaattaacacaagaggtgcccgagaatatcatgtccagttttttagcaaccagccagagagggcgtgggttcatgaaaaacgggtacgagagtataaaggtcataaacagtatgaagaattactggctgaggcaaccaaacaagccagcaatcactctgagaaacaaaagattcggaaaccccgacctcagagagaacgtgctcagtgggatattggcattgcccatgcagagaaagcattgaaaatgactcgagaagaaagaatagaacagtatacttttatttacattgataaacagcctgaagaggctttatcccaagcaaaaaagagtgttgcctccaaaaccgaagttaaaaaaacccgacgaccaagatctgtgctgaatactcagccagaacagaccaatgcaggggaggtggcctcctcactctcaagtactgaaattcggagacatagccagaggcggcacacaagtgcggaagaggaagagccaccgcctgttaaaatagcctggaaaactgcggcagcaaggaaatccttaccagcttccattacgatgcacaaagggagcctggatttgcagaagtgtaacatgtctccagttgtgaaaattgaacaagtgtttgctcttcagaatgctacaggggatgggaaatttatcgatcaatttgtttattcaacaaagggaattggtaacaaaacagaaataagtgtcagggggcaagacaggcttataatttctacaccaaaccagagaaatgaaaagccaacgcagagtgtatcatctcctgaagcaacatctggttctacaggctcagtagaaaagaagcaacagagaagatcaattagaactcgttctgaatcagagaaatccactgaggttgtgccaaagaagaagatcaaaaaggagcaggttgaaacagttcctcaggctacagtgaagactggattacagaaagggtcggcggaccggggagtgcagggctctgtcagattcagtgacagctccgtctccgcagcgattgaggaaactgtggactgagattcctgtacaatttcatcccagaaactccagacttgtagtctccatgcaagatttctttgtcggcggcttgataaacagtttctttgttttcgattttgatttcgccaatcatcattattggcattttcctgcctggtttcttcttcaagactctgaacaattgctttaacagtcaaatgatttttttttttcggtttgagctggatgggtacagcttaaatcatgggtccagcctaaaaaccaccatttaacttacactgatcaatttcaacatggactgtttttggttttttgtttttaaataaagcatcattaatgcacatctgcaggggtttgccaaacagcccaaactgtatacattacaatcattaaaagttcttattttttttaatattagtgccgttatcatggagaacagcatgacagctgtctttggcagtctgtcatttttctagcattttcagaaactcatcggaaatggcggtacctgtgtttcccttcgaaagcctctcagtacagcactcctgttcctctgttaaaactccttgttaatccagtgatcttttaggccaaggaaatattttgtgatggtgttctgggtccatacaccagcaatgaaggagatagatttgtgtacttgtgttttttaatcagcattaacatgggcaggcaccctcatttatagatgtcaggaaacattcagtgaaaaacttgtagaatgggatgtgataacgaggttccagtaatctgagcagtctaacgaggcccacctcctccaccacagaacgtggctatgttccaagtgctactctcactcagcctgttgcggatcttcatggcctcaggagacttgtttctccatgggctcttctggactgcacacttccaccatagcttgctgggttgatctagatgtctgtttgttgtatggaaattttgggggaaaaaatccaaaacacaaactgtgggttgaaatattaaccgtctccttggttccttggtattcaccgtgcctgatctgcacatttcatcgtggctgtttctgtatagcctatactgcattagcccaagagattgttgctttgtaactttttgcactattgttttggctggatttgtattacacacagttttaaaaaaaacaattccacactattctctgccttttttttcctatttatttcttcccgcacaaattccacatagaggccttcccatccagctctacgtgatttggctgcacttgaacactgattgtccatttacagccctcagcaatgtgccttctaaatggcatgacatatgtagatgtgctgcagcgcttgttaatggtcacaataaatgccacttcaccaaggaagtctcagatgaacaattatgaacatccaaattttattggggggcaataatcaactgaattgcaaaatttgggggaaaatggcactatccgtgtacgaatcgaatacaaatcaaagatttgtcacatccctaataaaaacaagatggagatgtctctgcaaccatatttgtaagcta

SEQ ID NO: 4 - protein, 645 aa, Homo sapiens
MDFSFSFMQGIMGNTIQQPPQLIDSANIRQEDAFDNNSDIAEDGGQTPYEATLQQGFQYPATTEDLPPLTNGYPSSISVYETQTKYQSYNQYPNGSANGFGAVRNFSPTDYYHSEIPNTRPHEILEKPSPPQPPPPPSVPQTVIPKKTGSPEIKLKITKTIQNGRELFESSLCGDLLNEVQASEHTKSKHESRKEKRKKSNKHDSSRSEERKSHKIPKLEPEEQNRPNERVDTVSEKPREEPVLKEEAPVQPILSSVPTTEVSTGVKFQVGDLVWSKVGTYPWWPCMVSSDPQLEVHTKINTRGAREYHVQFFSNQPERAWVHEKRVREYKGHKQYEELLAEATKQASNHSEKQKIRKPRPQRERAQWDIGIAHAEKALKMTREERIEQYTFIYIDKQPEEALSQAKKSVASKTEVKKTRRPRSVLNTQPEQTNAGEVASSLSSTEIRRHSQRRHTSAEEEEPPPVKIAWKTAAARKSLPASITMHKGSLDLQKCNMSPVVKIEQVFALQNATGDGKFIDQFVYSTKGIGNKTEISVRGQDRLIISTPNQRNEKPTQSVSSPEATSGSTGSVEKKQQRRSIRTRSESEKSTEVVPKKKIKKEQVETVPQATVKTGLQKGSADRGVQGSVRFSDSSVSAAIEETVD

SEQ ID NO: 5 - DNA, 20 bp, Artificial Sequence
  PCR primer
gcaaattccatggcaccgtc

SEQ ID NO: 6 - DNA, 19 bp, Artificial Sequence
  PCR primer
tcgccccacttgattttgg

SEQ ID NO: 7 - DNA, 20 bp, Artificial Sequence
  PCR primer
tgggaacaagagggcatctg

SEQ ID NO: 8 - DNA, 22 bp, Artificial Sequence
  PCR primer
ccaccactgcatcaaattcatg

SEQ ID NO: 9 - DNA, 23 bp, Artificial Sequence
  PCR primer
tcgaagcagctcttgtgtctaag

SEQ ID NO: 10 - DNA, 24 bp, Artificial Sequence
  PCR primer
tttggaccacaccaaatcaccaac

SEQ ID NO: 11 - DNA, 24 bp, Artificial Sequence
  PCR primer
aatatgactccttgctggagcagg

SEQ ID NO: 12 - DNA, 24 bp, Artificial Sequence
  PCR primer
atttcaacaggtggtctttgtctc

SEQ ID NO: 13 - DNA, 24 bp, Artificial Sequence
  PCR primer
agaacgtgctcagtgggatattgg

SEQ ID NO: 14 - DNA, 24 bp, Artificial Sequence
  PCR primer
tgcttgggataaagcctcttcagg

SEQ ID NO: 15 - DNA, 25 bp, Artificial Sequence
  PCR primer
caagccagcaatcactctgagaaac

SEQ ID NO: 16 - DNA, 25 bp, Artificial Sequence
  PCR primer
tatactgttctattctttcttctcg

SEQ ID NO: 17 - DNA, 21 bp, Artificial Sequence
  siRNA
gcagcacgacuucuucaagtt

SEQ ID NO: 18 - DNA, 21 bp, Artificial Sequence
  siRNA
cuugaagaagucgugcugctt

SEQ ID NO: 19 - DNA, 21 bp, Artificial Sequence
  siRNA
gugcgcugcuggugccaactt

SEQ ID NO: 20 - DNA, 21 bp, Artificial Sequence
  siRNA
guuggcaccagcagcgcactt

SEQ ID NO: 21 - RNA, 19 nt, Artificial Sequence
  siRNA
auccgcgcgauaguacgua

SEQ ID NO: 22 - RNA, 19 nt, Artificial Sequence
  siRNA
uacguacuaucgcgcggau

SEQ ID NO: 23 - RNA, 19 nt, Artificial Sequence
  siRNA
uuacgcguagcguaauacg

SEQ ID NO: 24 - RNA, 19 nt, Artificial Sequence
  siRNA
cguauuacgcuacgcguaa

SEQ ID NO: 25 - RNA, 19 nt, Artificial Sequence
  siRNA
uauucgcgcguauagcggu

SEQ ID NO: 26 - RNA, 19 nt, Artificial Sequence
  siRNA
accgcuauacgcgcgaaua

SEQ ID NO: 27 - DNA, 21 bp, Artificial Sequence
  siRNA
cagaucuacacagcggauatt

SEQ ID NO: 28 - DNA, 21 bp, Artificial Sequence
  siRNA
uauccgcuguguagaucugtt

SEQ ID NO: 29 - DNA, 19 bp, Artificial Sequence
  siRNA
cagatctacacagcggata

SEQ ID NO: 30 - DNA, 21 bp, Artificial Sequence
  siRNA
guuaauuggcauauggaautt

SEQ ID NO: 31 - DNA, 21 bp, Artificial Sequence
  siRNA
auuccauaugccaauuaactt

SEQ ID NO: 32 - DNA, 19 bp, Artificial Sequence
  siRNA
gttaattggcatatggaat

SEQ ID NO: 33 - DNA, 21 bp, Artificial Sequence
  siRNA
cucacaaauggguauccautt

SEQ ID NO: 34 - DNA, 21 bp, Artificial Sequence
  siRNA
auggauacccauuugugagtt

SEQ ID NO: 35 - DNA, 19 bp, Artificial Sequence
  siRNA
ctcacaaatgggtatccat

SEQ ID NO: 36 - DNA, 22 bp, Artificial Sequence
  siRNA
guacugaaauucggagagcatt

SEQ ID NO: 37 - DNA, 21 bp, Artificial Sequence
  siRNA
ugucuccgaauuucaguactt

SEQ ID NO: 38 - DNA, 19 bp, Artificial Sequence
  siRNA
gtactgaaattcggagaca

SEQ ID NO: 39 - DNA, 7219 bp, Homo sapiens
ggaccccggcaagcccgcgcacttggcaggagctgtagctaccgccgtccgcgcctccaaggtttcacggcttcctcagcagagactcgggctcgtccgccatgtccgccgcagacgaggttgacgggctgggcgtggcccggccgcactatggctctgtcctggataatgaaagacttactgcagaggagatggatgaaaggagacgtcagaacgtggcttatgagtacctttgtcatttggaagaagcgaagaggtggatggaagcatgcctaggggaagatctgcctcccaccacagaactggaggaggggcttaggaatggggtctaccttgccaaactggggaacttcttctctcccaaagtagtgtccctgaaaaaaatctatgatcgagaacagaccagatacaaggcgactggcctccactttagacacactgataatgtgattcagtggttgaatgccatggatgagattggattgcctaagattttttacccagaaactacagatatctatgatcgaaagaacatgccaagatgtatctactgtatccatgcactcagtttgtacctgttcaagctaggcctggcccctcagattcaagacctatatggaaaggttgacttcacagaagaagaaatcaacaacatgaagactgagttggagaagtatggcatccagatgcctgcctttagcaagattgggggcatcttggctaatgaactgtcagtggatgaagccgcattacatgctgctgttattgctattaatgaagctattgaccgtagaattccagccgacacatttgcagctttgaaaaatccgaatgccatgcttgtaaatcttgaagagcccttggcatccacttaccaggatatactttaccaggctaagcaggacaaaatgacaaatgctaaaaacaggacagaaaactcagagagagaaagagatgtttatgaggagctgctcacgcaagctgaaattcaaggcaatataaacaaagtcaatacattttctgcattagcaaatatcgacctggctttagaacaaggagatgcactggccttgttcagggctctgcagtcaccagccctggggcttcgaggactgcagcaacagaatagcgactggtacttgaagcagctcctgagtgataaacagcagaagagacagagtggtcagactgaccccctgcagaaggaggagctgcagtctggagtggatgctgcaaacagtgctgcccagcaatatcagagaagattggcagcagtagcactgattaatgctgcaatccagaagggtgttgctgagaagactgttttggaactgatgaatcccgaagcccagctgccccaggtgtatccatttgccgccgatctctatcagaaggagctggctaccctgcagcgacaaagtcctgaacataatctcacccacccagagctctctgtcgcagtggagatgttgtcatcggtggccctgatcaacagggcattggaatcaggagatgtgaatacagtgtggaagcaattgagcagttcagttactggtcttaccaatattgaggaagaaaactgtcagaggtatctcgatgagttgatgaaactgaaggctcaggcacatgcagagaataatgaattcattacatggaatgatatccaagcttgcgtggaccatgtgaacctggtggtgcaagaggaacatgagaggattttagccattggtttaattaatgaagccctggatgaaggtgatgcccaaaagactctgcaggccctacagattcctgcagctaaacttgagggagtccttgcagaagtggcccagcattaccaagacacgctgattagagcgaagagagagaaagcccaggaaatccaggatgagtcagctgtgttatggttggatgaaattcaaggtggaatctggcagtccaacaaagacacccaagaagcacagaagtttgccttaggaatctttgccattaatgaggcagtagaaagtggtgatgttggcaaaacactgagtgcccttcgctcccctgatgttggcttgtatggagtcatccctgagtgtggtgaaacttaccacagtgatcttgctgaagccaagaagaaaaaactggcagtaggagataataacagcaagtgggtgaagcactgggtaaaaggtggatattattattaccacaatctggagacccaggaaggaggatgggatgaacctccaaattttgtgcaaaattctatgcagctttctcgggaggagatccagagttctatctctggggtgactgccgcatataaccgagaacagctgtggctggccaatgaaggcctgatcaccaggctgcaggctcgctgccgtggatacttagttcgacaggaattccgatccaggatgaatttcctgaagaaacaaatccctgccatcacctgcattcagtcacagtggagaggatacaagcagaagaaggcatatcaagatcggttagcttacctgcgctcccacaaagatgaagttgtaaagattcagtccctggcaaggatgcaccaagctcgaaagcgctatcgagatcgcctgcagtacttccgggaccatataaatgacattatcaaaatccaggcttttattcgggcaaacaaagctcgggatgactacaagactctcatcaatgctgaggatcctcctatggttgtggtccgaaaatttgtccacctgctggaccaaagtgaccaggattttcaggaggagcttgaccttatgaagatgcgggaagaggttatcaccctcattcgttctaaccagcagctggagaatgacctcaatctcatggatatcaaaattggactgctagtgaaaaataagattacgttgcaggatgtggtttcccacagtaaaaaacttaccaaaaaaaataaggaacagttgtctgatatgatgatgataaataaacagaagggaggtctcaaggctttgagcaaggagaagagagagaagttggaagcttaccagcacctgttttatttattgcaaaccaatcccacctatctggccaagctcatttttcagatgccccagaacaagtccaccaagttcatggactctgtaatcttcacactctacaactacgcgtccaaccagcgagaggagtacctgctcctgcggctctttaagacagcactccaagaggaaatcaagtcgaaggtagatcagattcaagagattgtgacaggaaatcctacggttattaaaatggttgtaagtttcaaccgtggtgcccgtggccagaatgccctgagacagatcttggccccagtcgtgaaggaaattatggatgacaaatctctcaacatcaaaactgaccctgtggatatttacaaatcttgggttaatcagatggagtctcagacaggagaggcaagcaaactgccctatgatgtgacccctgagcaggcgctagctcatgaagaagtgaagacacggctagacagctccatcaggaacatgcgggctgtgacagacaagtttctctcagccattgtcagctctgtggacaaaatcccttatgggatgcgcttcattgccaaagtgctgaaggactcgttgcatgagaagttccctgatgctggtgaggatgagctgctgaagattattggtaacttgctttattatcgatacatgaatccagccattgttgctcctgatgcctttgacatcattgacctgtcagcaggaggccagcttaccacagaccaacgccgaaatctgggctccattgcaaaaatgcttcagcatgctgcttccaataagatgtttctgggagataatgcccacttaagcatcattaatgaatatctttcccagtcctaccagaaattcagacggtttttccaaactgcttgtgatgtcccagagcttcaggataaatttaatgtggatgagtactctgatttagtaaccctcaccaaaccagtaatctacatttccattggtgaaatcatcaacacccacactctcctgttggatcaccaggatgccattgctccggagcacaatgatccaatccacgaactgctggacgacctcggcgaggtgcccaccatcgagtccctgataggggaaagctctggcaatttaaatgacccaaataaggaggcactggctaagacggaagtgtctctcaccctgaccaacaagttcgacgtgcctggagatgagaatgcagaaatggatgctcgaaccatcttactgaatacaaaacgtttaattgtggatgtcatccggttccagccaggagagaccttgactgaaatcctagaaacaccagccaccagtgaacaggaagcagaacatcagagagccatgcagagacgtgctatccgtgatgccaaaacacctgacaagatgaaaaagtcaaaatctgtaaaggaagacagcaacctcactcttcaagagaagaaagagaagatccagacaggtttaaagaagctaacagagcttggaaccgtggacccaaagaacaaataccaggaactgatcaacgacattgccagggatattcggaatcagcggaggtaccgacagaggagaaaggccgaactagtgaaactgcaacagacatacgctgctctgaactctaaggccaccttttatggggagcaggtggattactataaaagctatatcaaaacctgcttggataacttagccagcaagggcaaagtctccaaaaagcctagggaaatgaaaggaaagaaaagcaaaaagatttctctgaaatatacagcagcaagactacatgaaaaaggagttcttctggaaattgaggacctgcaagtgaatcagtttaaaaatgttatatttgaaatcagtccaacagaagaagttggagacttcgaagtgaaagccaaattcatgggagttcaaatggagacttttatgttacattatcaggacctgctgcagctacagtatgaaggagttgcagtcatgaaattatttgatagagctaaagtaaatgtcaacctcctgatcttccttctcaacaaaaagttctacgggaagtaattgatcgtttgctgccagcccagaaggatgaaggaaagaagcacctcacagctcctttctaggtccttctttcctcattggaagcaaagacctagccaacaacagcacctcaatctgatacactcccgatgccacatttttaactcctctcgctctgatgggacatttgttacccttttttcatagtgaaattgtgtttcaggcttagtctgacctttctggtttcttcattttcttccattacttaggaaagagtggaaactccactaaaatttctctgtgttgttacagtcttagaggttgcagtactatattgtaagctttggtgtttgtttaattagcaatagggatggtaggattcaaatgtgtgtcatttagaagtggaagctattagcaccaatgacataaatacatacaagacacacaactaaaatgtcatgttattaacagttattaggttgtcatttaaaaataaagttcctttatatttctgtcccatcaggaaaactgaaggatatggggaatcattggttatcttccattgtgtttttctttatggacaggagctaatggaagtgacagtcatgttcaaaggaagcatttctagaaaaaaggagataatgtttttaaatttcattatcaaacttgggcaattctgtttgtgtaactccccgactagtggatgggagagtcccattgctaaaattcagctactcagataaattcagaatgggtcaaggcacctgcctgtttttgttggtgcacagagattgacttgattcagagagacaattcactccatccctatggcagaggaatgggttagccctaatgtagaatgtcattgtttttaaaactgttttatatcttaagagtgccttattaaagtatagatgtatgtcttaaaatgtgggtgataggaattttaaagatttatataatgcatcaaaagccttagaataagaaaagctttttttaaattgctttatctgtatatctgaactcttgaaacttatagctaaaacactaggatttatctgcagtgttcagggagataattctgcctttaattgtctaaaacaaaaacaaaaccagccaacctatgttacacgtgagattaaaaccaattttttccccattttttctccttttttctcttgctgcccacattgtgcctttattttatgagccccagttttctgggcttagtttaaaaaaaaaatcaagtctaaacattgcatttagaaagcttttgttcttggataaaaagtcatacactttaaaaaaaaaaaaaactttttccaggaaaatatattgaaatcatgctgctgagcctctattttctttctttgatgttttgattcagtattcttttatcataaatttttagcatttaaaaattcactgatgtacattaagccaataaactgctttaatgaataacaaactatgtagtgtgtccctattataaatgcattggagaagtatttttatgagactctttactcaggtgcatggttacagcccacagggaggcatggagtgccatggaaggattcgccactacccagaccttgttttttgttgtattttggaagacaggttttttaaagaaacattttcctcagattaaaagatgatgctattacaactagcattgcctcaaaaactgggaccaaccaaagtgtgtcaaccctgtttccttaaaagaggctatgaatcccaaaggccacatccaagacaggcaataatgagcagagtttacagctcctttaataaaatgtgtcagtaattttaaggtttatagttccctcaacacaattgctaatgcagaatagtgtaaaatgcgcttcaagaatgttgatgatgatgatatagaattgtggctttagtagcacagaggatgccccaacaaactcatggcgttgaaaccacacagttctcattactgttatttattagctgtagcattctctgtctcctctctctcctcctttgaccttctcctcgaccagccatcatgacatttaccatgaatttacttcctcccaagagtttggactgcccgtcagattgttgctgcacatagttgcctttgtatctctgtatgaaataaaaggtcatttgttcatgttaaaaaaaaa

SEQ ID NO: 40 - protein, 1657 aa, Homo sapiens
MSAADEVDGLGVARPHYGSVLDNERLTAEEMDERRRQNVAYEYLCHLEEAKRWMEACLGEDLPPTTELEEGLRNGVYLAKLGNFFSPKVVSLKKIYDREQTRYKATGLHFRHTDNVIQWLNAMDEIGLPKIFYPETTDIYDRKNMPRCIYCIHALSLYLFKLGLAPQIQDLYGKVDFTEEEINNMKTELEKYGIQMPAFSKIGGILANELSVDEAALHAAVIAINEAIDRRIPADTFAALKNPNAMLVNLEEPLASTYQDILYQAKQDKMTNAKNRTENSERERDVYEELLTQAEIQGNINKVNTFSALANIDLALEQGDALALFRALQSPALGLRGLQQQNSDWYLKQLLSDKQQKRQSGQTDPLQKEELQSGVDAANSAAQQYQRRLAAVALINAAIQKGVAEKTVLELMNPEAQLPQVYPFAADLYQKELATLQRQSPEHNLTHPELSVAVEMLSSVALINRALESGDVNTVWKQLSSSVTGLTNIEEENCQRYLDELMKLKAQAHAENNEFITWNDIQACVDHVNLVVQEEHERILAIGLINEALDEGDAQKTLQALQIPAAKLEGVLAEVAQHYQDTLIRAKREKAQEIQDESAVLWLDEIQGGIWQSNKDTQEAQKFALGIFAINEAVESGDVGKTLSALRSPDVGLYGVIPECGETYHSDLAEAKKKKLAVGDNNSKWVKHWVKGGYYYYHNLETQEGGWDEPPNFVQNSMQLSREEIQSSISGVTAAYNREQLWLANEGLITRLQARCRGYLVRQEFRSRMNFLKKQIPAITCIQSQWRGYKQKKAYQDRLAYLRSHKDEVVKIQSLARMHQARKRYRDRLQYFRDHINDIIKIQAFIRANKARDDYKTLINAEDPPMVVVRKFVHLLDQSDQDFQEELDLMKMREEVITLIRSNQQLENDLNLMDIKIGLLVKNKITLQDVVSHSKKLTKKNKEQLSDMMMINKQKGGLKALSKEKREKLEAYQHLFYLLQTNPTYLAKLIFQMPQNKSTKFMDSVIFTLYNYASNQREEYLLLRLFKTALQEEIKSKVDQIQEIVTGNPTVIKMVVSFNRGARGQNALRQILAPVVKEIMDDKSLNIKTDPVDIYKSWVNQMESQTGEASKLPYDVTPEQALAHEEVKTRLDSSIRNMRAVTDKFLSAIVSSVDKIPYGMRFIAKVLKDSLHEKFPDAGEDELLKIIGNLLYYRYMNPAIVAPDAFDIIDLSAGGQLTTDQRRNLGSIAKMLQHAASNKMFLGDNAHLSIINEYLSQSYQKFRRFFQTACDVPELQDKFNVDEYSDLVTLTKPVIYISIGEIINTHTLLLDHQDAIAPEHNDPIHELLDDLGEVPTIESLIGESSGNLNDPNKEALAKTEVSLTLTNKFDVPGDENAEMDARTILLNTKRLIVDVIRFQPGETLTEILETPATSEQEAEHQRAMQRRAIRDAKTPDKMKKSKSVKEDSNLTLQEKKEKIQTGLKKLTELGTVDPKNKYQELINDIARDIRNQRRYRQRRKAELVKLQQTYAALNSKATFYGEQVDYYKSYIKTCLDNLASKGKVSKKPREMKGKKSKKISLKYTAARLHEKGVLLEIEDLQVNQFKNVIFEISPTEEVGDFEVKAKFMGVQMETFMLHYQDLLQLQYEGVAVMKLFDRAKVNVNLLIFLLNKKFYGK

SEQ ID NO: 41 - DNA, 7218 bp, Homo sapiens
gccccgcatcgtgcccggccccgtcgcggagatcccggacgaccgtcgcgggttgatggtcgcattccagatgtaaacagcttcagaagcctgacggtcatatggtagaatcactgtggactgagacccacctttctagacctgaagcccaggaggaggaagaggaggctggttggtaccatgggcataatgctctgaatcctagtctctcacctagtatgtgagcagtccctgcagatggcccatttggagatcttgacaaagcctcttctgtttccaatggggtttttggcgcattctcacagacttagatgaaactgtgatggccaccgcagggggcaggtgctgacatcgtccccagccctgtggctgttcatccggacatcatttccaacctcaatatctaaatgccacagtgctcttggagcaagttgggctggggaccactgttgccttttaagaccataaaaccatgggaaacgcagaaagtcaacatgtagagcacgagttttatggagaaaagcatgccagcctggggcgcaagcacacttcccgctccctgcgcctctcgcacaagacgcggaggaccaggcacgcttcctcggggaaggtgatccacaggaactccgaagtgagcacccgatccagcagcacccccagcatcccccagtccctggctgaaaatggcctggagcccttctcccaagatggtaccctagaagacttcgggagccccatctgggtggaccgagtggacatgggcttgagacctgtgtcttacactgactcttctgtcactcccagcgtagacagcagcatcgtcctcacagcagcctctgtgcagagcatgccagacactgaggagagcaggctttacggggatgacgctacatatttggctgagggaggcaggaggcagcattcctatacatccaatgggcccactttcatggagacggcgagctttaagaagaaacgctccaaatctgcagacatctggcgggaggacagcctggaattctcactctctgatctgagccaagaacatttaacaagcaacgaagaaatcttgggttccgccgaagagaaggactgcgaggaggctcgggggatggaaacgcgggcgagtccgcggcagctcagcacctgtcagagagccaattccttgggtgacttgtatgctcagaaaaactctggagtgacagcaaacggggggccggggagcaaatttgcaggctactgtcggaatttggtgtctgatattcccaatcttgcaaaccataagatgccaccagctgctgctgaagagactcctccgtacagtaattataacacacttccctgtaggaaatctcactgtctctctgaaggtgccaccaacccacaaattagccatagcaacagcatgcaaggcagaagagctaaaacaactcaggatgttaatgcaggcgagggcagtgagtttgcagacagtgggattgaaggggccactaccgacacggacctcctgtccaggcgatctaatgccaccaactccagctactcacccaccacaggccgggcctttgtgggcagcgacagcggcagcagctccaccggggatgcggctcgtcagggggtgtacgagaacttccggcgggagctggagatgagcaccaccaacagcgagagcctggaggaggccggctcggcgcacagcgatgagcagagcagcggcaccctgagctctccgggccagtcggacatcctgctgaccgccgcacagggcacggtgcgcaaggccggcgccctggccgtcaagaacttcctggtgcacaagaagaacaagaaggtggagtcagccacccggaggaagtggaagcactactgggtgtccctgaaaggatgcacgctatttttctacgagagcgacggcaggtctgggatagaccacaacagcatccccaaacacgccgtctgggtggagaacagcattgtgcaggcggtgcctgagcaccccaagaaggactttgtcttctgcctcagcaattccctgggtgatgccttcctttttcagaccactagccagacggagcttgaaaactggatcaccgccatccactctgcctgcgccactgcggtcgcgaggcaccaccacaaggaagacacgctccgactcctgaaatcagagatcaaaaaactggaacagaagattgacatggatgaaaagatgaagaaaatgggtgaaatgcagctgtcttcagtcactgactcaaagaaaaagaaaacaatattagatcagatctttgtctgggagcaaaatctcgagcagttccaaatggacctgtttcgtttccgctgttatttagccagccttcagggtggggagctgccaaaccccaaaaggcttctcgcttttgcaagtcgaccaacgaaagtggccatgggccgccttggaatcttttcggtatcatcgtttcatgccctggtggcagcacgcactggtgaaactggagtgagaagacgtactcaggccatgtccagatccgcgagcaagcgaaggagcaggttttcttctctgtggggtctggatactacctccaaaaagaagcagggacggccaagcatcaatcaggtgtttggagagggaaccgaagctgtaaagaaatctttagagggaatatttgatgacattgttccagatggcaagagggagaaagaagtggtcttacctaacgttcaccagcacaaccctgactgcgacatttgggtccacgagtatttcactccatcctggttctgtctgcccaataatcagcctgccctgacggtcgtccggccaggcgacactgcacgggacaccctggagctgatttgcaagacacatcaactggatcattctgctcattacctgcgcctgaaatttctaatagaaaacaaaatgcagctctatgttccacagcccgaggaagacatctatgagctgctgtacaaagaaattgaaatctgtccaaaagtcactcagagcatccacattgagaagtcagatacagctgctgatacttacgggttttcactttcttctgtggaagaagatggtattcgaaggctgtacgtgaatagtgtgaaggaaaccggtttagcttccaagaaaggcctgaaagcaggagatgagattcttgagatcaataatcgtgctgctgacgccctgaactcttctatgctcaaagatttcctctcacagccctcgctgggcctcctggtgaggacctaccccgagctggaggaaggagtggagctgctggaaagcccgccccaccgagtggacggccctgccgaccttggcgagagccccctcgcctttctcaccagcaacccagggcacagcctttgcagcgagcagggcagcagtgctgagaccgctccagaggagaccgaggggccagacttggaatcctcagatgagactgatcacagcagcaagagtacagaacaggtggccgcattttgccgcagtttgcatgagatgaacccctctgaccagagcccatctcctcaggactccacggggcctcagctggcgaccatgagacaactctcggatgcagataagctgcgcaaggtgatctgcgagctcctggagacggagcgcacctacgtgaaggatttaaactgtcttatggagagatacctaaagcctcttcaaaaagaaacttttctcacccaggatgagcttgacgtgctttttggaaatttaacggaaatggtagagtttcaagtagaattccttaaaactctagaagatggagtgagactggtacctgatttggaaaagcttgagaaggttgatcaatttaagaaagtgctgttctctctggggggatcattcctgtattatgctgaccgcttcaagctctacagtgccttctgcgccagccacacaaaagttcccaaggtcctggtgaaagccaagacagacacggctttcaaggcattcttggatgcccagaacccgaagcagcagcactcatccacgctggagtcgtacctcatcaagcccatccagaggatcctcaagtacccacttctgctcagggagctgttcgccctgaccgatgcggagagcgaggagcactaccacctggacgtggccatcaagaccatgaacaaggttgccagtcacatcaatgagatgcagaaaatccatgaagagtttggggctgtgtttgaccagctgattgctgaacagactggtgagaaaaaagaggttgcagatctgagcatgggagacctgcttttgcacactaccgtgatctggctgaacccgccggcctcgctgggcaagtggaaaaaggaaccagagttggcagcattcgtcttcaaaactgctgtggtccttgtgtataaagatggttccaaacagaagaagaaacttgtaggatctcacaggctttccatttatgaggactgggaccccttcagatttcgacacatgatccccacggaagcgctgcaggttcgagctttggcgagtgcagatgcagaggcaaatgccgtgtgtgaaattgtccatgtaaaatccgagtctgaagggaggccggagagggtctttcacttgtgctgcagctccccagagagccgaaaggatttcctaaaggctgtgcattcaatcctgcgtgataagcacagaagacagctcctcaaaaccgagagccttccctcatcccagcaatatgtcccttttggaggcaaaagattgtgtgcactgaagggggccaggccggccatgagcagggcagtgtctgccccaagcaagtctcttgggaggaggaggcggcggctggctcgaaacaggtttaccattgattctgatgccgtctccgcaagcagcccggagaaagagtcccagcagccccccggtggtggggacactgaccgatgggtagaggagcagtttgatcttgctcagtatgaggagcaagatgacatcaaggagacagacatcctcagtgacgatgatgagttctgtgagtccgtgaagggtgcctcagtggacagagacctgcaggagcggcttcaggccacctccatcagtcagcgggaaagaggccggaaaaccctggatagtcacgcgtcccgcatggcacagctcaagaagcaagctgccctgtcggggatcaatggaggcctggagagcgcaagcgaggaagtcatttgggttaggcgtgaagactttgccccctccaggaaactgaacactgagatctgactgcgtcacctgccccgtagagaatgtgtgtagatacttcctgccctaactctgcccaccctcctgtaccgtcgacaagaatgtccccttaggtcgcgctcttgcacacacggttttggcagctgacttggttctgaagccatgtagccacccaactttgtcattttcaacaacatcagaaagaattgatcagaatcccaaataagcttgagtcctatcttctgtatattactaagggcttttatttattctcaataaatcagggcctgaacaattaaaagaaaaaagattctatagcactggaaagcaaatcaccccaggagttaacggatgtacaacagattaatttaagggatagtagcacacacacgatccttctatctgaaatcagtctcctagctggggaaacctctttcacacacaaaatgaaatgtgtacagcttgccgtgttctgactgtacccttccctcttccatgtctgagaatctccgtgtattttaagaatgtgtgaggagagggtggcgattcatgtttcaatgagcctctttttttttttccttcctgttttggtctatggctggtcttactctgtgtccatgttcggaagctctagttttgcatagaattatagagatgccaaactctttgaaaagagatccaaatttatcgcttgagagaaagaaaagaaacactattttttgtattttacctgagatacaggggcacaaatagatgagaattttacagtgttagtgtatgtatccctgagcctaaaaaatgaggatataaccttttacagagagagtgaggcgtggtggttttatatttatatatgaaaggccagcaagctcatgcgaaggatatacttttcttccaaaaagcggattttttttttttaatgtttgaatctatatttgagatgggagtttggttggattaaacatgacaccccggtgggcggtgtgtgtgtctgttgcacatggcagggaggggagcctccttctcatggggttgccatggtgatcattggtttttccatcaaaattgcatcttcatccatagattaccttccccttccctgacagtccataaccaaacctttaaacagaacaacctctttaaaaacttctcttgtgtttaacactttcttcatgccaacgaaacagggtaaacatgctcaaaacattaacagtctaaacagatatccaaatactaagaagaaaaacaagttatagcactttcaattttttttttttttttaaaaaaaggtttatagctttttcttttcccatgtcacaatgtccacttcctaagaagggtttaaaatactatgaaaactttctttttggggaaaatatctatttggtgtttgacacatcagtaggtactttaaagacctgaattttatagtagctttaggagttatattttataaaaatcagttatgactttatatttccagacaatagagagttcagtacatcatgctcttgtgcctctgcctgcttttcctgcgttcccaccctgtattccccccgcctttcgggtttccagggcttcgagcttgatcttttgaaagttttattctattaaatttttgctatatcttctggttttctgaaaaagctttagaatggtttctataccctttgtatcactgcatttttccatatcatctccggttcgatcgcgtccagatggaaaacggaagcagaggcttctaatcgtcgcatttactggctccagtgcaacacatccatctgaaaacactcggaagtctggtgcttggagagggtgccattgtctcttgtacataaggtcatgacgtgtctatgtcaaaagttcttatatatttcttttataagctgaaagaaggtctatttttatgtttttaggtctatgaatggaacgttgtaaatgcttgtcaaacaataaaaataacgaaaagtgaaaaaaaaaaaaaaaaaaaa

SEQ ID NO: 42 - protein, 1591 aa, Homo sapiens
MGNAESQHVEHEFYGEKHASLGRKHTSRSLRLSHKTRRTRHASSGKVIHRNSEVSTRSSSTPSIPQSLAENGLEPFSQDGTLEDFGSPIWVDRVDMGLRPVSYTDSSVTPSVDSSIVLTAASVQSMPDTEESRLYGDDATYLAEGGRRQHSYTSNGPTFMETASFKKKRSKSADIWREDSLEFSLSDLSQEHLTSNEEILGSAEEKDCEEARGMETRASPRQLSTCQRANSLGDLYAQKNSGVTANGGPGSKFAGYCRNLVSDIPNLANHKMPPAAAEETPPYSNYNTLPCRKSHCLSEGATNPQISHSNSMQGRRAKTTQDVNAGEGSEFADSGIEGATTDTDLLSRRSNATNSSYSPTTGRAFVGSDSGSSSTGDAARQGVYENFRRELEMSTTNSESLEEAGSAHSDEQSSGTLSSPGQSDILLTAAQGTVRKAGALAVKNFLVHKKNKKVESATRRKWKHYWVSLKGCTLFFYESDGRSGIDHNSIPKHAVWVENSIVQAVPEHPKKDFVFCLSNSLGDAFLFQTTSQTELENWITAIHSACATAVARHHHKEDTLRLLKSEIKKLEQKIDMDEKMKKMGEMQLSSVTDSKKKKTILDQIFVWEQNLEQFQMDLFRFRCYLASLQGGELPNPKRLLAFASRPTKVAMGRLGIFSVSSFHALVAARTGETGVRRRTQAMSRSASKRRSRFSSLWGLDTTSKKKQGRPSINQVFGEGTEAVKKSLEGIFDDIVPDGKREKEVVLPNVHQHNPDCDIWVHEYFTPSWFCLPNNQPALTVVRPGDTARDTLELICKTHQLDHSAHYLRLKFLIENKMQLYVPQPEEDIYELLYKEIEICPKVTQSIHIEKSDTAADTYGFSLSSVEEDGIRRLYVNSVKETGLASKKGLKAGDEILEINNRAADALNSSMLKDFLSQPSLGLLVRTYPELEEGVELLESPPHRVDGPADLGESPLAFLTSNPGHSLCSEQGSSAETAPEETEGPDLESSDETDHSSKSTEQVAAFCRSLHEMNPSDQSPSPQDSTGPQLATMRQLSDADKLRKVICELLETERTYVKDLNCLMERYLKPLQKETFLTQDELDVLFGNLTEMVEFQVEFLKTLEDGVRLVPDLEKLEKVDQFKKVLFSLGGSFLYYADRFKLYSAFCASHTKVPKVLVKAKTDTAFKAFLDAQNPKQQHSSTLESYLIKPIQRILKYPLLLRELFALTDAESEEHYHLDVAIKTMNKVASHINEMQKIHEEFGAVFDQLIAEQTGEKKEVADLSMGDLLLHTTVIWLNPPASLGKWKKEPELAAFVFKTAVVLVYKDGSKQKKKLVGSHRLSIYEDWDPFRFRHMIPTEALQVRALASADAEANAVCEIVHVKSESEGRPERVFHLCCSSPESRKDFLKAVHSILRDKHRRQLLKTESLPSSQQYVPFGGKRLCALKGARPAMSRAVSAPSKSLGRRRRRLARNRFTIDSDAVSASSPEKESQQPPGGGDTDRWVEEQFDLAQYEEQDDIKETDILSDDDEFCESVKGASVDRDLQERLQATSISQRERGRKTLDSHASRMAQLKKQAALSGINGGLESASEEVIWVRREDFAPSRKLNTEI

SEQ ID NO: 43 - DNA, 5263 bp, Homo sapiens
cggggggggccgcgccgtgctagccgttgggcctgcctcggaggaggcgtcgccgccgccgctgccgctgccggcgccgttgccgctgccgggaaacacaaggaaagggaaccagcgcagcgtggcgatgggcgggggtagagccccgccggagaggctgggcggctgccggtgacagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcggaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtgagcagtctgcccacgcagaggacgcacgctcgctgccatcaccgctgggtggttttttccccctaactttttacttagcctttttggtttgtgtccccacccccacctcctcaccccctttccagttcttcttcaggcccctcccagacgcaccccagcggcccctgcagcccctgcctccagcctccagcctcacctttgtgcccagactcgcatttggaagactccacctcccgcccaggcctgggctgttgggcggttggagattcaggttttaatccacacaagccccagtgaggggtgaagcatggcgcctggggcctgcctgagtttctggcctgggtgtcgtgctggtgtctgcctccgcgctgctgcatctggacgaaggctgccttctggtgggacgcgacacccggcagacagtggtgctgccttccaggccccgtggcctaggctcggagtggccaggcacggggcggtccaatcccccacccgctgtccccctatgggggcagaaaagcaataatgtccaggggcaggcaggggcccttgggagctgcagggctgggggttagggctgctccctggtgaatggagtcagatcctaggatctgtaccatggggaaccaggagtggccgggctgggtgccgcctcctggtccggcctcctccccaccaaactgtcctcaccctatggatgaggcaggaggaacatttggggccaaacctgcctgcctcccagccccgtgccttactagggcttccttccagctggccttacctcccgctggaccctgggcctggcctggccccactgggggctatgggctgggctcaccctctcctctgcgggggtggagggccaccagccttggctgttacaatcttacaccggacagtattgggccccatggacttggtcagggaggggtgggggtgggcatctctggtacctattggggtggggggcctctgaaaagggaggctcctaggcccccctcacccctccctctccccagggccccacgttctgcagccttaaggttgaacatgagtgcacgtccatgtcagtgctgtgggactcctgtgcgtgcctcggactgcgtgtgtcggcgggacgcaggcacacgtgggtgtgtgtgcatgtgtgtttgtgtgagggcagcgtgtcctccagtgtgcatggtgtgtgggcttgggccccatccctggcccgagcatttcatcctgtgggggaggggtgctgacctagtgggaggagccccactgtgatccatgagctgccctgcccacgcctcccctccctgtagcaacacctctgggtgtttggagtttagcttttgtgggtttgctctccctatcccatctcctgtactacacagttcatggcagggtggggaggggtggggttggttcgggtgggtgagggtctttttcctctgtgtgcgatgttgttatctgacagttctccgtccctactggcctttctcctcgtcttcatatttgtacggtacaagcaataaagacactcatttcagaccagggcccagcctgcactcacgccagcccaaccactctgggctttgccttggtgatggagtcagacccctgggccccagctcctcctgtactagccgttcccttcagcaaggagggcactgagctcagggtgagggcagctggggtgtgtgcaggagctcaggctggagagggtgggtggagctggtgctgtggggctgaggggtatgggaaggctccccgcatgtgggggtggggtggacagagaccactccaggccctcagtgctgcttaggctaagagaggtggggtggagggacagggctggaagatctgggtagcccagaatgaggagggtgcctgtgctgtcactgaatgagagggagtggttcattccacccggctgccgagcctcagaggggggcattcctatcctgccccacctccctgtttatgctgccacctggaagccttgaggcccccaaattccagtacagacccagtggtgtgttcatggtggcgtggttgctgtcacctgggagctcctgagcgtttggttagaaccctgttcagcttggggtcagccctcccctagtcactgccctttagcctggatgtgtctgggcccctgcacttcccgtgcttgagtcacgtggctgcatggccgggcgctggccggatggaacacctcccccagcaagggaccagggaccagagccctggcctgccctgctgagccctgctgtgcagagggcctggcacagatgaatttgagattttgccgcaaggtgttagcacttcacacccattgagtctttgagattttaagtgaatgtaagcagaaaaagtcagatccaatttacagaaatcagagttagctacagctaggactcgtttggttggggttttttagtttgtctttctaaagtcatgtggaccttaatttaattacaaaagtctaccctggtggtcataaaataggcaggcctatgaagaaaggccttttactcttccatctcatcccagccccgagttgacccacgttgctgctcctcacaccatggtgatgcaggtctcgtagtgtgggcacaggcctggctacctcatctttttagtgcctctctcctcttccacaggatggggtcccacagctgcagcagctggccccgtagttgagcatgtgtggttatcctgtagagcttttcccaagaagggtgtttgaacttagagtcttaataaaatcttaccaaataaattttgagtagaataatcgtcttttgcaatgtacattttaaaaatttcacacattcttttttgtatataaagaacagtgactgggcacagtggctcatgcctgtaatcccagcaatttgggaggccgaggcgggcgggtctcttgaggccaggggttcgagaccagcctgggcatcatagggagaccttcatctctacaaaaaatacaaaaattagctgggcatggtggtgcatgcctgcaatcccagctaacttggaaggctgaggtgaggtgggaagatcacttgagcccaggagtttgaggctgcagtgagctatgattgcggcactgcactgcagcctgggacaatgagactgtgtctctaaaaataaaaaaaaaaaaaacatgatacatgctattaaaaaagacagcaaagcaggagtataagaaaggaaattcacccgaggtcgcagggccttgagtactcattttggtgctgattacctctctgcaaatggacacggcatcataaattggtagtttcctgctctttttgtgtaatcttttccagttaatgtgaagcctctgggggctgccctcgtgcactgatggttgtgtggagtcgggggcggcagtgcgattcccttttagctgctgcatggggggaactcaggctttccagctgcttcctggggttccatggggtagacccctcaaccgcttcagctgccccgttaacaggaattgacttggtttcgtttggtgctaccagcagtcctgtaataaactagctatccatctgta

SEQ ID NO: 44 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 45 - DNA, 3415 bp, Homo sapiens
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 46 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 47 - DNA, 4304 bp, Homo sapiens
cacacggactacaggggagttttgttgaagttgcaaagtcctggagcctccagagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtgcccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaaccccaaaaaaaaaaaaaaaa

SEQ ID NO: 48 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 49 - DNA, 5431 bp, Homo sapiens
gggggctttgtgcgcggcggcggcgggagaggcggcggcggcggccagcacggaggcggaggccgagggggctgtgcacaggtcgccgcggagaggcgtgcgaattccgagccgagcgccgaggaccgtgctacccaggccgggctgccagccgcaggctcctctctggcagcagcggcggcgcggcgacccccgtccctcggcctccccttcccatcccacctcccgagccttcctcttcccgcagcacgcccggcccggcccggccgtggccctcctcagtgccggccgccatggcagaggcgtccggcgcggggaaaatctagcccggggatttcatgcggcctagctcggttccgcctcctcctcgcgcggccccagcggctgcccgcaccccagccccactccgggcctccgtgtctctcctgtgatcgcactgacacggccggggggttagaatggaacaaactgaaggcccgatgagagaaagggaaagttaaggatgctggagcagaacaatggatttctctttctctttcatgcaagggatcatgggaaacacaattcagcaaccacctcaactcattgactccgccaacatccgtcaggaggatgcctttgataacaacagtgacattgctgaagatggtggccagacaccatatgaagctactttgcagcaaggctttcagtacccagctacaacagaagatcttcctccactcacaaatgggtatccatcatcaatcagtgtgtatgaaactcaaaccaaataccagtcatataatcagtatcctaatgggtcagccaatggctttggtgcagttagaaactttagccccactgactattatcattcagaaattccaaacacaagaccacatgaaattctggaaaaaccttcccctccacagccaccacctcctccttcggtaccacaaactgtgattccaaagaagactggctcacctgaaattaaactaaaaataaccaaaactatccagaatggcagggaattgtttgagtcttccctttgtggagaccttttaaatgaagtacaggcaagtgagcacacgaaatcaaagcatgaaagcagaaaagaaaagaggaaaaaaagcaacaagcatgactcatcaagatctgaagagcgcaagtcacacaaaatccccaaattagaaccagaggaacaaaatagaccaaatgagagggttgacactgtatcagaaaaaccaagggaagaaccagtactaaaagaggaagccccagttcagccaatactatcttctgttccaacaacggaagtgtccactggtgttaagtttcaggttggcgatcttgtgtggtccaaggtgggaacctatccttggtggccttgtatggtttcaagtgatccccagcttgaggttcatactaaaattaacacaagaggtgcccgagaatatcatgtccagttttttagcaaccagccagagagggcgtgggttcatgaaaaacgggtacgagagtataaaggtcataaacagtatgaagaattactggctgaggcaaccaaacaagccagcaatcactctgagaaacaaaagattcggaaaccccgacctcagagagaacgtgctcagtgggatattggcattgcccatgcagagaaagcattgaaaatgactcgagaagaaagaatagaacagtatacttttatttacattgataaacagcctgaagaggctttatcccaagcaaaaaagagtgttgcctccaaaaccgaagttaaaaaaacccgacgaccaagatctgtgctgaatactcagccagaacagaccaatgcaggggaggtggcctcctcactctcaagtactgaaattcggagacatagccagaggcggcacacaagtgcggaagaggaagagccaccgcctgttaaaatagcctggaaaactgcggcagcaaggaaatccttaccagcttccattacgatgcacaaagggagcctggatttgcagaagtgtaacatgtctccagttgtgaaaattgaacaagtgtttgctcttcagaatgctacaggggatgggaaatttatcgatcaatttgtttattcaacaaagggaattggtaacaaaacagaaataagtgtcagggggcaagacaggcttataatttctacaccaaaccagagaaatgaaaagccaacgcagagtgtatcatctcctgaagcaacatctggttctacaggctcagtagaaaagaagcaacagagaagatcaattagaactcgttctgaatcagagaaatccactgaggttgtgccaaagaagaagatcaaaaaggagcaggttgaaacagttcctcaggctacagtgaagactggattacagaaaggtgccagcgagatttcagattcctgtaaacctctaaagaaaaggagtcgcgcctcaactgatgtagaaatgactagttcagcatacagagacacatctgactccgattctagaggactgagtgacctgcaggtaggctttggaaagcaagtagatagcccttcagctactgcagatgcagacgtttctgatgtgcagtccatggattcaagtttgtcgagaagaggcactggaatgagtaagaaggacactgtatgtcagatttgtgaaagctctggtgactctctgattccttgtgagggagagtgctgcaaacactttcacctggagtgcctgggattggcatcacttcctgatagcaagttcatctgcatggaatgtaaaactgggcagcacccatgtttttcgtgtaaagtgtctggtaaagatgtgaagcgttgttctgttggtgcttgtgggaaattttatcatgaagcctgtgtccgcaaattccccactgccatctttgaatcaaaaggattccgctgtcctcagcactgctgctctgcctgctctatggagaaagatatccacaaagcaagtaaaggccgcatgatgagatgtttaagatgtccagttgcctatcactctggagatgcttgcattgcggccggaagcatgttagtatcctcctacattctcatctgtagtaatcattccaaacggagcagtaattcttctgctgtaaatgtaggcttttgtttcgtttgtgccagagggctgatagttcaggaccattcagaccccatgttcagttcatatgcctataagtcccactacctactgaatgaatcaaatcgtgctgagttgatgaaattacctatgattccttcttcgtcagcttccaaaaagaaatgtgagaaaggtggaagattgctctgctgtgaatcgtgcccagcttccttccacccggaatgcctaagcatagaaatgccagaaggctgctggaattgtaatgactgtaaagctggcaagaaactacattacaagcagattgtttgggtcaaattgggaaattacagatggtggccagcagagatctgcaaccccaggtctgtgccactgaacatccagggccttaaacatgacttgggggacttccctgtattcttctttggttctcatgactactactgggtacaccagggcagagtgttcccttatgttgaaggagacaaaagctttgctgaagggcagactagtattaacaagaccttcaaaaaggcactggaagaagctgcaaaacgtttccaggaattgaaagcacaaagagaaagtaaagaagccctagagattgaaaaaaactcaagaaaaccccctccctacaaacacatcaaagctaacaaagtaataggaaaggtgcagatccaggttgctgacctgtcagagattccccgctgtaactgcaagccagctgatgaaaacccttgtggcttggaatcggagtgcctgaacagaatgttgcagtatgaatgccacccgcaggtgtgcccagctggagatcgttgtcagaaccagtgctttacaaagagactataccctgatgcagagatcatcaaaacggagcggagaggctggggcctcaggaccaaaaggagcattaagaagggtgaatttgtaaatgaatacgtcggtgaattaattgatgaagaagaatgcagattgcgaatcaagcgagcccacgagaacagtgtaactaatttttatatgttaactgttaccaaggaccgtataattgatgccggcccaaaaggaaattattctcgcttcatgaaccacagttgtaatcccaactgtgaaacacaaaagtggacagtgaatggagatgttcgagtgggactatttgctctctgtgatattcctgcagggatggagttaacatttaattataacctagattgtctgggcaacggcagaacggagtgccactgtggagcagataactgcagtggttttctaggagtgcggccaaagtcggcatgtgcgtcaacaaatgaagagaaggcaaaaaatgctaagttaaaacagaagagacgaaagatcaaaacagaaccaaagcagatgcatgaagattactgttttcaatgtggagatggtggagagctggtcatgtgtgacaaaaaagactgtcccaaagcataccacctcctatgccttaacctgactcagccaccatatggaaagtgggagtgtccgtggcatcagtgcgatgagtgcagcagtgcagctgtttccttctgtgaattctgtccacattcattttgtaaagatcatgaaaagggggccctggttccctctgcactggaaggccgcctctgctgctcggaacatgaccccatggctcctgtgtcaccagaatactggagcaagataaaatgtaaatgggaatcacaagatcatggagaagaagtaaaagaataaatgtgtggtgtcccctcctttctatttaagtgaaaaaagcaaatagatcatgcatttaaaaagaagagactgctacagtgcatacagcctttgccatcggaactgccttattaaagcaaaaatgggaaaccagttcatgcaggcagaagcagttggtggtgtctggtttttgtttgatttggttggtttgggattcttttgtggagggttaaattcccttggtcttttcttgccttttattgtgcttcagtgccattgcagcttgaaaaagaaatgtttttgctgttaaaataagaacaaagagaaaagtaagttttgttaatgagataaatttaaagtctaagatgtgttccttggttgtataaagcaaaagtagccatcattcctttatttattttcatttttaggaatttcaagaagtgtagttcaatagtctaatcaagtgtgtgtgtgttttaagtaggaatctgagaaagccctctaggaaagggtatgataagctttatatacctctttactgagcagtaggtaggctcacttctctttcccttcaaaatgcttttcataggcttagagaagggctctatggaagtattaaa

SEQ ID NO: 50 - protein, 1437 aa, Homo sapiens
MDFSFSFMQGIMGNTIQQPPQLIDSANIRQEDAFDNNSDIAEDGGQTPYEATLQQGFQYPATTEDLPPLTNGYPSSISVYETQTKYQSYNQYPNGSANGFGAVRNFSPTDYYHSEIPNTRPHEILEKPSPPQPPPPPSVPQTVIPKKTGSPEIKLKITKTIQNGRELFESSLCGDLLNEVQASEHTKSKHESRKEKRKKSNKHDSSRSEERKSHKIPKLEPEEQNRPNERVDTVSEKPREEPVLKEEAPVQPILSSVPTTEVSTGVKFQVGDLVWSKVGTYPWWPCMVSSDPQLEVHTKINTRGAREYHVQFFSNQPERAWVHEKRVREYKGHKQYEELLAEATKQASNHSEKQKIRKPRPQRERAQWDIGIAHAEKALKMTREERIEQYTFIYIDKQPEEALSQAKKSVASKTEVKKTRRPRSVLNTQPEQTNAGEVASSLSSTEIRRHSQRRHTSAEEEEPPPVKIAWKTAAARKSLPASITMHKGSLDLQKCNMSPVVKIEQVFALQNATGDGKFIDQFVYSTKGIGNKTEISVRGQDRLIISTPNQRNEKPTQSVSSPEATSGSTGSVEKKQQRRSIRTRSESEKSTEVVPKKKIKKEQVETVPQATVKTGLQKGASEISDSCKPLKKRSRASTDVEMTSSAYRDTSDSDSRGLSDLQVGFGKQVDSPSATADADVSDVQSMDSSLSRRGTGMSKKDTVCQICESSGDSLIPCEGECCKHFHLECLGLASLPDSKFICMECKTGQHPCFSCKVSGKDVKRCSVGACGKFYHEACVRKFPTAIFESKGFRCPQHCCSACSMEKDIHKASKGRMMRCLRCPVAYHSGDACIAAGSMLVSSYILICSNHSKRSSNSSAVNVGFCFVCARGLIVQDHSDPMFSSYAYKSHYLLNESNRAELMKLPMIPSSSASKKKCEKGGRLLCCESCPASFHPECLSIEMPEGCWNCNDCKAGKKLHYKQIVWVKLGNYRWWPAEICNPRSVPLNIQGLKHDLGDFPVFFFGSHDYYWVHQGRVFPYVEGDKSFAEGQTSINKTFKKALEEAAKRFQELKAQRESKEALEIEKNSRKPPPYKHIKANKVIGKVQIQVADLSEIPRCNCKPADENPCGLESECLNRMLQYECHPQVCPAGDRCQNQCFTKRLYPDAEIIKTERRGWGLRTKRSIKKGEFVNEYVGELIDEEECRLRIKRAHENSVTNFYMLTVTKDRIIDAGPKGNYSRFMNHSCNPNCETQKWTVNGDVRVGLFALCDIPAGMELTFNYNLDCLGNGRTECHCGADNCSGFLGVRPKSACASTNEEKAKNAKLKQKRRKIKTEPKQMHEDYCFQCGDGGELVMCDKKDCPKAYHLLCLNLTQPPYGKWECPWHQCDECSSAAVSFCEFCPHSFCKDHEKGALVPSALEGRLCCSEHDPMAPVSPEYWSKIKCKWESQDHGEEVKE

SEQ ID NO: 51 - DNA, 3256 bp, Homo sapiens
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 52 - DNA, 3720 bp, Homo sapiens
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 53 - DNA, 21 bp, Artificial Sequence
  PCR primer
cagtaacgtcacacggactac

SEQ ID NO: 54 - DNA, 21 bp, Artificial Sequence
  PCR primer
cgctccctcgcgctcttctgc

SEQ ID NO: 55 - DNA, 21 bp, Artificial Sequence
  PCR primer
cccctcttccctggcggggag

SEQ ID NO: 56 - DNA, 21 bp, Artificial Sequence
  PCR primer
gcccaaaagccatccctgagg

SEQ ID NO: 57 - DNA, 21 bp, Artificial Sequence
  PCR primer
gtggtctccccaggctgcgtg

SEQ ID NO: 58 - DNA, 21 bp, Artificial Sequence
  PCR primer
aggggtgcagggggccccgtc

SEQ ID NO: 59 - DNA, 21 bp, Artificial Sequence
  PCR primer
gcagtcgctgagattctttgg

SEQ ID NO: 60 - DNA, 21 bp, Artificial Sequence
  PCR primer
accacgagaaggggtgactgg

SEQ ID NO: 61 - DNA, 21 bp, Artificial Sequence
  PCR primer
cgcccctgtgcgcccggaatg

SEQ ID NO: 62 - DNA, 21 bp, Artificial Sequence
  PCR primer
tcagcgactgcatcttctttc